(19) United States
(12) Patent Application Publication
(10) Pub. No.: US 2011/0256560 A1
(43) Pub. Date: Oct. 20, 2011

(54) METHODS AND COMPOSITIONS FOR THE DETECTION OF OVARIAN CANCER

(75) Inventors: Eleftherios P. Diamandis, Toronto (CA); Cynthia Kuk, Richmond Hill (CA)

(73) Assignee: UNIVERSITY HEALTH NETWORK, Toronto, ON (CA)

(21) Appl. No.: 13/125,272

(22) PCT Filed: Oct. 20, 2009
(86) PCT No.: PCT/CA09/01479

§ 371 Date: Jun. 29, 2011

Related U.S. Application Data
(60) Provisional application No. 61/106,864, filed on Oct. 20, 2008.

(51) Int. Cl. G01N 33/566

(52) U.S. Cl. 435/7.92; 436/501

(57) ABSTRACT

Provided is a method of screening for, diagnosing or detecting ovarian cancer in a subject comprising (a) determining a level of mdogen-2 in a test sample from the subject, and (b) comparing the level of mdogen-2 in the test sample with a control, where detecting an increase in the level of mdogen-2 in the test sample compared to the control is indicative of ovarian cancer in the subject.

### FIELD

The disclosure relates to methods and compositions for screening for, detecting or diagnosing ovarian cancer and/or prognosing ovarian cancer survival.

### BACKGROUND

Accounting for approximately 3% of all new cancer cases in 2008¹ with a 1 in 59 (1.7%) lifetime probability of developing the disease, ovarian cancer is the most lethal gynecological malignancy deeming 5-6% of all cancer deaths¹. Hidden deep within the pelvis, ovarian cancer is relatively asymptomatic in early stages and due to the lack of adequate screening, ovarian cancer has resulted in the majority of cases being presented with late stage disease in association with a low 5-year survival rate of 25-40%. When presented at an early stage, the 5-year survival rate exceeds 90% and most patients are cured by surgery alone². While the most widely used serum marker for ovarian cancer is carbohydrate antigen 125 (CA125), its utility as a screening marker is limited due to its high false positive rates and elevation in other malignancies such as uterine, fallopian, colon and gastric cancer^{3, 4} as well as in non-malignant conditions such as pregnancy and endometriosis⁵. These reasons alone demonstrate the need and immediate benefit in using biomarkers with increased sensitivity and specificity for early diagnosis, prognosis or monitoring of ovarian cancer.

Many advanced stage ovarian cancer patients exhibit rapid growth of intraperitoneal tumors along with abdominal distention as a result of accumulation of ascites fluid in the peritoneal cavity. Mechanistically, ascites formation occurs as malignant cells secrete proteins, growth factors and cytokines that cause neovascularization, angiogenesis, increased fluid filtration and/or lymphatic obstruction⁶⁻⁸ resulting in the build-up of serum-like fluid within the abdomen. Body fluids have been shown to be excellent media for biomarker discoveryl¹⁰. Mass spectrometry has been widely used to identify the proteome of fluids¹³⁻¹⁶, and specifically, Gortzak-Uzan et al. have recently attempted to identify the proteome of ascites, both cellular and fluid fractions¹⁷.

Many ovarian cancer biomarkers are inadequate due to their relatively low diagnostic sensitivity and specificity. There is a need to discover and validate novel ovarian cancer biomarkers that are suitable for early diagnosis, monitoring and prediction of therapeutic response.

### SUMMARY OF THE DISCLOSURE

Biomarkers associated with ovarian cancer are described herein. The biomarkers are useful for diagnosing, monitoring therapeutic response and prognosing survival.

An aspect of the disclosure provides a method of screening for, diagnosing or detecting ovarian cancer or an increased likelihood of developing ovarian cancer in a subject comprising:
(a) determining a level of a biomarker in a test sample from the subject wherein the biomarker is selected from the biomarkers set out in Table 2; and
(b) comparing the level of the biomarker in the test sample with a control;

wherein detecting an altered level of the biomarker in the test sample compared to the control is indicative of whether the subject has or does not have ovarian cancer or an increased likelihood of developing ovarian cancer in the subject.

Another aspect provides a method for monitoring the therapeutic response of a subject with ovarian cancer comprising:
(a) determining a level of biomarker in a reference sample of the subject, the biomarker selected from the biomarkers set out in Table 2;
(b) determining the level of biomarker in a subsequent sample of the subject, the subsequent sample taken subsequent to the subject receiving a ovarian cancer treatment or therapy; and
(c) comparing the levels of the biomarker in the reference sample to the level of the biomarker in the subsequent sample,

wherein an altered level of the biomarker in the subsequent sample compared to the reference sample is indicative of therapeutic response.

A further aspect provides a method of prognosing survival in a subject with ovarian cancer comprising:
(a) determining a level of a biomarker in a test sample from the subject wherein the biomarker is selected from the biomarkers set out in Table 2; and
(b) comparing the level of the biomarker in the test sample with a control and/or a positive control;

wherein an altered level of the biomarker in the test sample compared to the control and/or positive control is indicative of the survival prognosis of the subject.

Another aspect provides a method of detecting relapse in a subject previously having ovarian cancer comprising:
(a) determining a level of a biomarker in a test sample from the subject wherein the biomarker is selected from the biomarkers set out in Table 2; and
(b) comparing the level of the biomarker in the test sample with a control;

wherein an increased level of the biomarker in the test sample compared to the control is indicative of relapse of ovarian cancer in the subject.

Yet a further aspect provides a method of identifying an ovarian cancer biomarker in a subject with ovarian cancer comprising:
(a) obtaining a test sample from the subject, wherein the test sample comprises ascites fluid or serum;
(b) gel filtering and/or centrifugal ultrafiltering the test sample;
(c) selecting fractions depleted of high molecular weight proteins;
(d) digesting the fractions;
(e) analyzing the digested fractions by mass spectrometry to identify a plurality of unique polypeptides;
(f) selecting one or more polypeptide(s) of the plurality of unique polypeptides as a candidate biomarker for validation; and
(g) validating an association of a polypeptide level of the collection of unique polypeptides with ovarian cancer or an increased likelihood of ovarian cancer;

wherein the association with ovarian cancer or the increased likelihood of ovarian cancer is indicative the polypeptide is an ascites or serum biomarker.

In certain embodiments of all the above methods, the biomarker is nidogen-2.

Compositions, immunoassays and kits are also provided.

Other features and advantages of the disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### DRAWINGS

Embodiments of the disclosure will now be described in relation to the drawings in which:

FIG. 1. Elution profile of total protein (♦) and KLK6 (▪) during 1 gel filtration. Fractions with molecular mass of ≦30 KDa (first vertical line) were collected and analyzed by LC-MS/MS. Fractions between the two vertical lines were re-chromatographed to remove additional high-abundance proteins. For more details, see text. Monomeric KLK6 (approximate molecular mass 30 KDa) elutes at fractions 37-38; the second peak likely represents fragmented KLK6.

FIG. 2. Ascites fluid fractionation protocol prior to LC-MS/MS analysis. For more details, see text. A, NH₄HCO₃ buffer gel filtration, SCX-LC-MS/MS. B, Phosphate/sulfate buffer gel filtration, LC-MS/MS. C, 50 KDa ultrafiltration, LC-MS/MS. D, 100 KDa ultrafiltration, SCX-LC-MS/MS. SCX; strong cation-exchange. Digestion was with trypsin.

FIG. 3. Number of proteins identified with each fractionation method. In total, 445 proteins were identified.

FIG. 4. Classification of 445 ascites proteins by subcellular localization.

FIG. 5. Selection of 52 candidate ovarian cancer biomarkers based on the criteria shown above and as described in text.

FIG. 6. Number of proteins identified with each buffer system. A total of 434 proteins were identified by performing size exclusion chromatography using phosphate/sulfate and ammonium bicarbonate (NH₄HCO₃) mobile phase.

FIG. 7. Number of proteins identified by each ultrafiltration device. A total of 144 proteins were identified by ultrafiltrating 15 mL of ascites using nominal mass cutoff devices of 50 KDa and 100 KDa.

FIG. 8. Overlap between the soluble ascites fluid subproteome and data of Gortzak-Uzan et al¹⁷.

FIG. 9. The concentration of nidogen-2 and CA125 in normal serum, benign gynecological conditions and ovarian carcinoma is shown in FIGS. 9A and 9B respectively. Both CA125 and nidogen-2 are elevated in ovarian cancer serum samples and not in normal and benign conditions.

FIG. 10. The correlation between nidogen-2 and CA125 is shown in normal (FIG. 10A), benign (FIG. 10B) and ovarian cancer (FIG. 10 C) sample sets.

FIG. 11. Nidogen-2 is shown to correlate with CA125 in cancer.

FIG. 12. Nidogen-2 and CA125 expression in serous cystadenocarcinoma of the ovary, mucinous cystadenocarcinoma of the ovary, endometrioid adenocarcinoma of the ovary and clear cell carcinoma of the ovary is shown in FIGS. 12A and 12B respectively. Both nidogen-2 and CA125 are elevated in patients with serous cystadenocarcinoma.

FIG. 13. Nidogen-2 and CA125 are both elevated in late stage (stage 3 and 4) ovarian cancer as opposed to early stage (stage 1 and 2). FIG. 13A and FIG. 13B shows nidogen-2 and CA125 expression in each stage respectively. FIG. 13C and FIG. 13D shows nidogen-2 and CA125 expression in early stage (stage 1 and 2) and late stage (stage 3 and 4) respectively.

FIG. 14. Receiver Operating Characteristic (ROC) curves for single marker of nidogen-2 or CA125 with estimated AUC (95% Cl). Normal patients versus ovarian cancer patients.

FIG. 15. Receiver Operating Characteristic (ROC) curves for single marker of nidogen-2 or CA125 with estimated AUC (95% Cl). Benign disease patients versus ovarian cancer patients.

### DESCRIPTION OF VARIOUS EMBODIMENTS

Described herein is an in-depth proteomic analysis of ovarian cancer ascites fluid. Size exclusion chromatography and ultrafiltration were used to remove high-abundance proteins with molecular mass ≧30 KDa. After trypsin digestion, the subproteome (≦30 KDa) of ascites fluid was determined by two-dimensional liquid chromatography-tandem mass spectrometry. Filtering criteria were used to select potential ovarian cancer biomarker candidates. By combining data from different size exclusion and ultrafiltration fractionation protocols, 445 proteins were identified from the soluble ascites fraction using a 2-D linear ion-trap mass spectrometer. Among these were 25 proteins previously identified as ovarian cancer biomarkers. After applying a set of filtering criteria to reduce the number of potential biomarker candidates, 52 proteins were identified. This proteomic approach for discovering novel ovarian cancer biomarkers is highly efficient since it was able to identify 25 known biomarkers and 52 candidate new biomarkers.

### I. DEFINITIONS AND ABBREVIATIONS

The term “additional biomarker” as used herein refers to a biomarker set out in Table 1, as well as other known ovarian cancer biomarkers such as CA125.

The term “agent” in the context “agent detects a biomarker” refers to any molecule including any chemical, nucleic acid, polypeptide or composite molecule and/or any composition that permits quantitative assessment of the biomarker level. For example the agent can comprise a detectable marker and a detection agent, such as an isolated polypeptide, peptide mimetic, or antibody, a nucleic acid such as an aptamer, and/or a chemical compound or composition that binds to, reacts with and/or responds to a biomarker in Table 2.

The term “altered level” as used herein refers to a difference in the level, or quantity, of a biomarker in a test sample that is measurable, including for example a difference in the level of expression, secretion, release, cleavage, shedding and/or level and type of post-translational modification of the biomarker compared to a control and/or reference sample associated with, for example, having ovarian cancer or an increased likelihood of developing ovarian cancer, a prognosis or treatment response. For example, the altered level is optionally a level statistically associated with a particular group or outcome, for example having ovarian cancer or not having ovarian cancer. The post-translational modifications may include for example differential glycosylation levels or different types of glycosylation e.g. O-linked glycosylation, N-linked glycosylation. The altered level can refer to an increase or decrease in the measurable polypeptide or fragment level of a given biomarker as measured by the amount of expressed, secreted, released, shed or modified polypeptide or fragment in a test sample as compared with the measurable expression level of a given biomarker in a control and/or previously taken sample. For example, in methods relating to screening for, diagnosing or detecting ovarian cancer, altered level refers to an increase in the level of a biomarker compared to a control, wherein the control corresponds to a biomarker level in a subject without ovarian cancer. In methods relating to monitoring therapeutic response, altered level can refer to a decrease or increase in the level of the biomarker in the subsequent sample compared to a reference sample, wherein a decrease is indicative of positive therapeutic response and/or an increase is indicative of a negative therapeutic response. The term can also refer to an increase or decrease in the measurable expression, secretion, release, shedding and/or post-translational modification level of a given biomarker in a test sample as compared with the measurable expression, secretion, release, shedding and/or post-translational modification level of a biomarker in a population of samples. As mentioned the altered level can refer to an increase or decrease in soluble biomarker, for example secretion, release, cleavage, shedding and/or post-translational modifications of a biomarker in a test sample, such as a serum sample, compared to a control or reference sample. For example the altered level can include an increased ratio of a post-translational modified biomarker compared to a control or reference sample, which is detectable in a test sample (e.g. antibody specific for modified form or detectable by size changes). For example, a biomarker level is altered if the ratio of the level in a test sample as compared with a control is greater than or less than 1.0 and/or if the ratio of the level in a reference sample as compared with a subsequent sample is greater than or less than 1.0. For example, a ratio of greater than 1, 1.2, 1.5, 1.7, 2, 3, 3, 5, 10, 12, 15, 20 or more, or a ratio less than 1, 0.8, 0.6, 0.4, 0.2, 0.1, 0.05, 0.001 or less. The altered expression, secretion, release, shedding and/or post-translational modification level when compared to a population average can for example be expressed using p-value. For instance, when using p-value, a biomarker is identified as having altered expression, secretion, release, cleavage, shedding and/or post-translational modification as between a first and second population when the p-value is less than 0.1, such as less than 0.05, 0.01, 0.005, and/or less than 0.001.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include Fab, Fab′, F(ab′)₂, scFv, dsFv, ds-scFv, dimers, minibodies, diabodies, and multimers thereof and bispecific antibody fragments. Antibodies can be fragmented using conventional techniques. For example, F(ab′)₂ fragments can be generated by treating the antibody with pepsin. The resulting F(ab′)₂ fragment can be treated to reduce disulfide bridges to produce Fab′ fragments. Papain digestion can lead to the formation of Fab fragments. Fab, Fab′ and F(ab′)₂, scFv, dsFv, ds-scFv, dimers, minibodies, diabodies, bispecific antibody fragments and other fragments can also be synthesized by recombinant techniques.

The term “ascites” and/or “ascitic fluid” as used herein refers to fluid and/or cells therein comprised accumulating in the peritoneal cavity. This fluid for example may be a symptom of ovarian cancer. A person skilled in the art would be familiar with methods for obtaining test samples comprising ascites. Further, ascites and/or ascitic fluid can be fractionated or separated into a cell free fraction e.g. “cell free ascites” (fluid fraction) or a fraction containing cells e.g. “ascites cell fraction”.

The term “biomarker” as used herein refers to a gene or gene product characteristic that can be measured and evaluated as an indicator of pathogenic processes relating to ovarian cancer, or pharmacological responses to an ovarian cancer therapeutic intervention. For example, biomarker refers to a gene product, such as a polypeptide or fragment, that is differentially expressed, secreted, released, cleaved, shed, and/or post-translationally modified in subjects with ovarian cancer as compared to subjects without ovarian cancer. Post-translational modifications can include for example differential biomarker glycosylation levels in subjects with and without ovarian cancer and/or different types of glycosylation e.g. O-linked glycosylation or N-linked glycosylation. The biomarkers of the disclosure include the biomarkers as set out in Table 2.

The term “soluble biomarker” as used herein refers to a polypeptide biomarker or fragment thereof that is detectable in a biological fluid such as ascites or blood or a fraction thereof, for example biomarker that is secreted, released, or shed from a cell for example an ovarian cancer cell, and detectable in for example serum.

The term “serologic biomarker” as used herein refers to a polypeptide biomarker or fragment thereof that is detectable in serum, for example biomarker that is secreted, released, or shed from a cell for example an ovarian cancer cell, and detectable in serum.

The phrase “benign conditions” or “benign gynecological disease” refers to a non-malignant condition that is not life threatening. For example, these conditions may include but not limited to uterine leiomyoma, adenomyosis and ovarian cyst.

The phrase “biomarker polypeptide”, “polypeptide biomarker” or “polypeptide product of a biomarker” refers to a proteinaceous biomarker gene product or fragment thereof. For example, a biomarker polypeptide refers to a Table 2 polypeptide biomarker or fragment thereof that is for example, increased in samples from subjects with ovarian cancer.

The term “BSA” refers to bovine serum albumin.

The term “CA125” refers to carbohydrate antigen 125.

The term “control” as used herein refers to a sample, and/or a biomarker level, numerical value and/or range (e.g. control range) corresponding to the biomarker level in such a sample, taken from or associated with a subject or a population of subjects (e.g. control subjects) who are known as not having ovarian cancer. For example, it has been determined herein that subjects without ovarian cancer exhibit a particular range of nidogen-2 biomarker level (e.g. control level). Test subjects having an increased nidogen-2 biomarker level have or are more likely to have ovarian cancer.

The control can be for example, a level of biomarker in a sample of a subject which is compared to a level of biomarker in a control, wherein the control comprises a control sample or a numerical value derived from a sample, optionally the same sample type as the sample (e.g. both the sample and the control are serum samples or both the sample and the control sample are plasma samples), from a subject known as not having ovarian cancer. Where the control is a numerical value or range, the numerical value or range is a predetermined value or range that corresponds to a level of the biomarker or range of levels of the biomarker in a group of subjects known as not having ovarian cancer (e.g. threshold or cutoff level; or control range). For example, the control can be a cut-off or threshold level. It is demonstrated herein that test subjects that have an increased level of biomarker above the cut-off or threshold level have or are more likely to have ovarian cancer.

The term “positive control” as used herein refers to a sample and/or biomarker level or numerical value corresponding to the biomarker level in a sample from a subject or a population of subjects (e.g. positive control subjects) who are known as having ovarian cancer. Where the positive control corresponds to a positive control sample or a numerical value or range corresponding to a biomarker level in a positive control sample e.g. from a subject or population with ovarian cancer, the positive control sample may optionally be obtained from a subject or subjects that has been treated (e.g. treated positive control) or that has not been treated (e.g. untreated positive control) for ovarian cancer.

The term “control level” refers to a biomarker level in a control sample or a numerical value corresponding to such a sample. Control level can also refer to for example a threshold, cut-off or baseline level of a biomarker in subjects without ovarian cancer, where levels above which are associated with the presence of ovarian cancer or for example a stage of ovarian cancer such as advanced ovarian cancer.

Similarly the term “positive control level” refers to a biomarker level in a positive control sample. Positive control level can also refer to a threshold, cut-off or baseline level of a biomarker in subjects with ovarian cancer, for example associated with a particular stage of ovarian cancer.

The term “control sample” or “positive control sample” as used herein refers to any biological fluid, cell or tissue sample from a control subject or positive control subject respectively e.g. a subject with known disease status and including a comparison sample, such as a sample prior to treatment, from the subject being tested, which can be assayed for biomarker levels. For example, the sample can comprise blood, serum, plasma, tumour biopsy ascitic fluid, sputum, urine, and/or bodily secretions.

The term “DTT” refers to dithiothreitol.

The term “ELISA” as used herein refers to enzyme-linked immunosorbent assay and includes for example indirect, sandwich and competitive ELISAs.

The phrase “false positive rate” as used herein refers to the proportion of negative instances that were erroneously reported as being positive. The false positive rate described herein (FPR) was calculated as: FPR=number of false proteins/(number of true proteins+number of false proteins) as identified by searching the mass spectrometry results against a concatenated database of reverse and forward sequence of the human genome. True proteins are the proteins correctly identified within the forward database while false proteins are the proteins incorrectly identified, as matched by sequences in the reverse database.

The term “GO” refers to the gene ontology, which provides a vocabulary for genes and gene products.

The term “hybridize” refers to the sequence specific non-covalent binding interaction with a complementary nucleic acid. The hybridization is conducted under appropriate stringency conditions such as high stringency conditions. Appropriate stringency conditions which promote hybridization are known to those skilled in the art, or can be found in Current Protocols in Molecular Biology, John Wiley & Sons, N.Y. (1989), 6.3.1 6.3.6. For example, 6.0× sodium chloride/sodium citrate (SSC) at about 45° C., followed by a wash of 2.0×SSC at 50° C. may be employed.

The term an “increased risk” or “increased likelihood of developing”, as used herein is used to mean that a test subject with increased levels of a biomarker in Table 2, for example an increased level of nidogen-2, has an increased chance of developing ovarian cancer, having recurrence or relapse or poorer survival relative to a control subject (e.g. a subject with control levels of a Table 2 biomarker, such as control serum levels). The increased risk for example may be relative or absolute and may be expressed qualitatively or quantitatively. For example, an increased risk may be expressed as simply determining the test subject's expression level for a given biomarker and placing the test subject in an “increased risk” category, based upon previous population studies. Alternatively, a numerical expression of the test subject's increased risk may be determined based upon biomarker level analysis. As used herein, examples of expressions of an increased risk include but are not limited to, odds, probability, odds ratio, p-values, attributable risk, relative frequency, positive predictive value, negative predictive value, and relative risk.

The term “IPI” refers to international protein index. Protein information can be accessed by many publicly available databases such as EMBL-EBI (http://www.ebi.ac.uk/IPI/IPIhelp.html).

The term “isolated polypeptide” as used herein refers to a proteinaceous agent, such as a peptide, polypeptide or protein, which is substantially free of cellular material or culture medium when produced recombinantly, or chemical precursors, or other chemicals, when chemically synthesized.

The term “isolated nucleic acid” as used herein refers to a nucleic acid substantially free of cellular material or culture medium when produced by recombinant DNA techniques, or chemical precursors, or other chemicals when chemically synthesized. An “isolated nucleic acid” is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded. The nucleic acid sequences contemplated by the disclosure include isolated nucleotide sequences which hybridize to a RNA product of a biomarker, nucleotide sequences which are complementary to a RNA product of a biomarker of the disclosure, nucleotide sequences which act as probes, or nucleotide sequences which are sets of specific primers for a biomarker set out in Table 2.

The term “KLK6” refers to kallikrein 6.

The term “level” as used herein refers to a quantity of biomarker that is detectable or measurable in a sample and/or control. The quantity is typically an extracellular quantity where extracellular can include cell associated product levels such as cell surface expression and/or cleaved, secreted, released or shed biomarker levels detected in a biological fluid such as serum. The quantity is for example a quantity of polypeptide or a subset thereof such as soluble biomarker, the quantity of nucleic acid, the quantity of a fragment (e.g. such as a shed or cleaved cell surface protein e.g. soluble biomarker), the quantity of complexed biomarker and/or the quantity of post-translational modified biomarker, such as the quantity of glycosylated, or phosphorylated biomarker. The level can alternatively include combinations thereof.

The term “monitoring therapeutic response” as used herein refers to assessing disease progression or lack thereof of a subject during the course of an ovarian cancer therapy and/or before and after an ovarian cancer treatment (e.g. before and after surgery). For example, the assessment can involve determining the level of a biomarker in a reference sample and at least one subsequent sample, wherein the at least one or more subsequent samples is taken after the initiation of a treatment or therapy. In another example, the assessment involves determining the level of a biomarker in a reference sample and several subsequent samples taken at intervals post treatment or therapy initiation. Therapeutic efficacy is determined if the level of the biomarker is altered over time. The reference sample is optionally taken before the initiation of treatment or therapy and/or after the initiation of treatment or therapy.

The term “MS/MS” refers to tandem mass spectrometry.

The term “NID2” or “nidogen-2” alternatively referred to “nidogen-2 precursor” and also known as “osteonidogen”, as used herein refers to an expression product or fragment thereof of the NID2 gene, such as a polypeptide expression product or fragment thereof, including mammalian NID2 including those deposited in Genbank NM_{—}007361.3 and PID g144953895 and/or sequences referred to herein as well as naturally occurring variants. The NID2 gene resides on locus 14q22.1 (chromosome 14).

The term “ovarian cancer” as used herein refers to dysregulated growth arising from an ovary, including for example surface epithelial-stromal tumours, sex cord-stromal tumors, and mixed tumours as well as ovarian cancer as a secondary cancer and low malignant potential ovarian cancer. Types of ovarian cancer also include for example epithelial ovarian cancer, sex cord-stromal cell ovarian cancer and germ cell ovarian cancer. Subtypes of these classes include clear cell carcinoma, serous such as serous cystadenocarcinoma, mucinous such as mucinous cystadenocarcinoma, endometrioid such as endometrioid adenocarcinoma, transitional cell (Brenner) or borderline ovarian tumors. Ovarian cancer comprises various stages including for example, early stage ovarian cancer and late stage ovarian cancer. “Early stage ovarian cancer” as used herein refers to stages wherein the subject has a 90% or greater 5-year survival upon treatment or stage 1 or 2 ovarian cancer as defined by histopathological analysis. The phrase “late stage ovarian cancer” as used herein refers to stages wherein the subject has an approximately 25-40% 5-year survival upon treatment or stage 3 or 4 ovarian cancer as defined by histopathological analysis. These stages are further defined by using criteria developed by international organizations such as FIGO (International Federation of Gynecology and Obstetrics).

The phrase “PIM assay” or product ion monitoring assay refers to an assay whereby an antibody is isolated, sample is applied for protein capture and the concentration of a select protein specific to the antibody is measured by mass spectrometry after trypsin digestion and identification of one or more peptides characteristic of the protein of interest.

The term “primer” as used herein refers to a nucleic acid sequence, whether occurring naturally as in a purified restriction digest or produced synthetically, which is capable of acting as a point of synthesis of when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less. The factors involved in determining the appropriate length of primer are readily known to one of ordinary skill in the art. The term “biomarker specific primers” as used herein refers a set of primers which can produce a double stranded nucleic acid product complementary to a portion of one or more RNA products of the biomarkers described herein or sequences complementary thereof.

The term “probe” as used herein refers to a nucleic acid sequence that will hybridize to a nucleic acid target sequence. In one example, the probe hybridizes to a RNA product of the biomarker of the disclosure or a nucleic acid sequence complementary to the RNA product of the biomarker of the disclosure. The length of probe depends on the hybridize conditions and the sequences of the probe and nucleic acid target sequence. In an embodiment, the probe is at least 8, 10, 15, 20, 25, 50, 75, 100, 150, 200, 250, 400, 500 or more nucleotides in length.

The term “prognosis” as used herein refers to an expected course of clinical disease. The prognosis provides an indication of disease progression and includes for example, an indication of likelihood of recurrence, metastasis, death due to disease, tumor subtype or tumor type. The prognosis can comprise a good prognosis which corresponds to a good clinical outcome relative to the spectrum of possible clinical outcomes for ovarian cancer, and a poor prognosis, which corresponds to a poor clinical outcome relative to the spectrum of possible clinical outcomes for ovarian cancer.

The phrase “prognosing survival” as used herein refers to identifying the likelihood of survival, such as disease free survival, and/or recurrence and/or death, and can comprise for example “good survival” or “poor survival”. As used herein “good survival” refers to an increased likelihood of disease free survival for at least 60 months, for example a 90% or greater likelihood of 5 year disease free survival. As used herein “poor prognosis” to an increased likelihood of relapse, recurrence, metastasis or death within 60 months, for example less than 25-40% likelihood of 5 year disease free survival.

The term “proteome” as used herein refers to the set of polypeptides expressed, secreted, released, cleaved, shed and/or otherwise present polypeptides, including post-translationally modified forms thereof, present in a sample type, such as ascitic fluid or serum, and/or refers to the set of polypeptides expressed, secreted, released, cleaved, shed and/or modified by a cell and/or tumour, for example an ovarian cancer cell and/or ovarian tumor.

The term “subproteome” refers to a subset of the set of polypeptides comprised in a proteome, for example, a subset detectable using a particular method such as liquid chromatography.

The term “reference sample” as used herein refers to a suitable comparison sample, obtained from the subject, for example before treatment and/or a previous time point.

The term “reference level” as used herein refers to a biomarker level corresponding to a suitable comparison sample, in the subject, for example before treatment and/or a previous time point.

The term “detection agent” refers to a molecule that selectively binds to, reacts with and/or responds to a biomarker such as an isolated polypeptide, nucleic acid, antibody and/or chemical compound.

The phrase “screening for, diagnosing or detecting ovarian cancer or an increased likelihood of developing ovarian cancer” refers to a method or process of determining if a subject has or does not have ovarian cancer, or has or does not have an increased risk of developing ovarian cancer. For example, detection of altered levels of a Table 2 biomarker compared to control is indicative that the subject has ovarian cancer or an increased risk of developing ovarian cancer.

The term “SCX” refers to strong cation exchange chromatography.

The term “sensitivity” as used herein means the percentage of subjects who have ovarian cancer who are identified by the assay as positive (e.g. biomarker level is above the cutoff point for the disorder).

The term “specificity” as used herein means the percentage of subjects who do not have ovarian cancer who are identified by the assay as negative (e.g. biomarker level is below the cutoff point) for the disorder.

The term “subject” as used herein refers to any member of the animal kingdom, preferably a human being.

The phrase “subject previously having ovarian cancer” refers to a subject who has been diagnosed with ovarian cancer, treated and whose cancer is in remission or not detectable.

The term “test sample” as used herein refers to any biological fluid, cell or tissue sample from a subject, which can be assayed for biomarker levels. For example, the sample can comprise blood, serum, plasma, tumour biopsy, tissue specimen, ascitic fluid, pleural effusions, tear drops, sputum, urine, and/or bodily secretions.

The term “therapeutic response” as used herein refers to any reaction or response in the subject precipitated or caused, directly or indirectly, by any therapy or treatment.

The phrase “therapy or treatment” as used herein, refers to an approach aimed at obtaining beneficial or desired results, including clinical results and includes medical procedures and applications including for example chemotherapy, pharmaceutical interventions, surgery, radiotherapy and naturopathic interventions as well as test treatments. The phrase “ovarian cancer therapy or treatment” refers to any approach including for example surgery, chemotherapy, preventive interventions, prophylactic interventions and test treatments aimed at alleviating or ameliorating one or more symptoms, diminishing the extent of, stabilizing, preventing the spread of, delaying or slowing the progression of, ameliorating or palliating and/or inducing remission of ovarian cancer and/or any associated complications thereof.

The phrase “treatment efficacy” and/or “positive therapeutic response”, as used herein refers to obtaining beneficial or desired clinical results which can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment efficacy” and/or positive therapeutic response” can also mean prolonging survival as compared to expected survival if not receiving treatment.

The term “treatment failure” and/or “negative therapeutic response” refers to not obtaining beneficial or desired clinical results including obtaining no therapeutic response and/or obtaining undesired results that exceed any positive therapeutic response, including enhancing disease spread and/or hastening death.

### II. METHODS

### a) Diagnosis, Monitoring Therapeutic Efficacy and Prognosing Survival

An aspect of the disclosure relates to methods of diagnosing ovarian cancer or an increased likelihood of developing ovarian cancer. Accordingly, an aspect of the disclosure provides a method of screening for, diagnosing or detecting ovarian cancer or an increased likelihood of developing ovarian cancer in a subject comprising:
(a) determining a level of a biomarker in a test sample from the subject wherein the biomarker is selected from the biomarkers set out in Table 2; and
(b) comparing the level of the biomarker in the test sample with a control;
   wherein detecting an altered level of the biomarker in the test sample compared to the control is indicative of whether the subject has ovarian cancer or a likelihood of developing ovarian cancer.

In an embodiment, the disclosure includes a method of screening for, diagnosing or detecting ovarian cancer or an increased likelihood of developing ovarian cancer in a subject comprising:
(a) determining a level of a biomarker in a test sample from the subject wherein the biomarker is selected from the biomarkers set out in Table 2; and
(b) comparing the level of the biomarker in the test sample with a control;
   wherein detecting an increased level of the biomarker in the test sample compared to the control is indicative the subject has ovarian cancer or an increased likelihood of developing ovarian cancer.

In another embodiment, detecting a decreased level of the biomarker in the test sample compared to the control is indicative the subject does not have ovarian cancer or an increased likelihood of developing ovarian cancer.

In an embodiment, the level of at least 2 biomarkers is determined and an increased level of at least 1 biomarker in the test sample compared to the control is indicative of ovarian cancer or an increased likelihood of developing ovarian cancer in the subject. In another embodiment, the level of at least 3, 4, 5, 6, 7, 8, 9, 10 or more biomarkers is determined and an increased level of at least 1 biomarker in the test sample compared to the control is indicative of ovarian cancer or an increased likelihood of developing ovarian cancer in the subject. In another embodiment, an increased level of at least 2 biomarkers in the test sample compared to the control is indicative of ovarian cancer or an increased likelihood of developing ovarian cancer in the subject.

The method in an embodiment comprises comparing the level of biomarker in the test sample to a positive control. An increased level compared to a control or positive control is indicative of ovarian cancer and/or an increased likelihood of developing ovarian cancer and a decreased level is indicative that the subject does not have ovarian cancer and/or an increased likelihood of developing ovarian cancer. In an embodiment, the control corresponds to a biomarker level in a control subject or a population of control subjects known to not have ovarian cancer, and detection of an increased level of the biomarker in the sample of the subject compared to the control is indicative the subject has ovarian cancer or an increased likelihood of developing ovarian cancer. The subject biomarker level can also be compared to a positive control. In an embodiment, the positive control corresponds to a biomarker level in a positive control subject or population of positive control subjects known to have ovarian cancer, and detection of a decreased level of the biomarker in the sample compared to the positive control (e.g. similar to a control level) is indicative the subject does not have ovarian cancer or an increased likelihood of developing ovarian cancer. In another example, detecting a similar or increased level of the biomarker compared to the positive control is indicative the subject has ovarian cancer or an increased likelihood of developing ovarian cancer. In another embodiment, where the control corresponds to a threshold level, below which is indicative of not having ovarian cancer and above which is indicative of having ovarian cancer, a decreased level of the biomarker in the sample compared to the control, is indicative that the subject does not have ovarian cancer and/or an increased likelihood of developing ovarian cancer and an increased level is indicative that the subject does have ovarian cancer and/or an increased likelihood of developing ovarian cancer.

The biomarker levels can be compared using the ratio of the level of expression of a given biomarker or biomarkers as compared with the expression level of the given biomarker or biomarkers of a control, wherein the ratio is not equal to 1.0. For example, a biomarker RNA, polypeptide or fragment is differentially expressed, secreted, released, cleaved, shed and/or post-translationally modified if the ratio of the level of biomarker in a test or reference sample as compared with a control or subsequent sample is greater than or less than 1.0. For example, a ratio of a biomarker level in a test subject sample to a biomarker level in a control greater than 1, 1.2, 1.5, 1.7, 2, 3, 3, 5, 10, 15, 20 or more is indicative that the subject has, for example, ovarian cancer or an increased risk of developing ovarian cancer, or a ratio of a biomarker level in a test subject sample compared to a positive control less than 1, 0.8, 0.6, 0.4, 0.2, 0.1, 0.05, 0.001 or less is indicative the subject does not have, for example ovarian cancer or an increased risk of developing ovarian cancer.

In another embodiment the altered levels are measured using p-value. For instance, when using p-value, a biomarker is identified as being differentially expressed, secreted, released, cleaved, shed and/or post-translationally modified as between a test and control population when the p-value is less than 0.1, preferably less than 0.05, more preferably less than 0.01, even more preferably less than 0.005, the most preferably less than 0.001.

In an embodiment, the altered level is an increased level, wherein an increased level is indicative for example, that the subject has ovarian cancer or an increased risk of developing ovarian cancer. In an embodiment, the ratio of the level of the biomarker in the test sample compared to the control is greater than 1, 1.2, 1.5, 1.7, 2, 3, 3, 5, 10, 12, 15, 20 or more. In another embodiment, the positive control is a sample of, or a numerical value that corresponds to, a subject with ovarian cancer, for example late stage ovarian cancer. In another embodiment, the altered level is a decreased level, wherein the decreased level for example compared to the positive control is indicative the subject does not have ovarian cancer or an increased risk of developing ovarian cancer. In an embodiment, the ratio of the level of the biomarker in the test sample compared to the control is less than 1, 0.8, 0.6, 0.4, 0.2, 0.1, 0.05, 0.001 or less.

The positive control can also correspond to a biomarker level in late stage ovarian cancer. Accordingly, in an embodiment, the positive control is a positive control sample of, or a numerical value that corresponds to, a subject with late stage ovarian cancer. In an embodiment, the altered level is a decrease in the level of the biomarker in the test sample compared to the positive control, wherein the decreased level is indicative the subject does not have late stage ovarian cancer or has a decreased risk of developing late stage ovarian cancer. In another embodiment, the altered level is an increase in the level of the biomarker in the test sample compared to the control, wherein the increased level is indicative the subject has late stage ovarian cancer or has an increased risk of developing late stage ovarian cancer.

Another aspect provides a method for monitoring the therapeutic response of a subject with ovarian cancer comprising:
(a) determining a level of biomarker in a reference sample of the subject, the biomarker selected from the biomarkers set out in Table 2;
(b) determining the level of biomarker in a subsequent sample of the subject, the subsequent sample taken subsequent to the subject receiving a ovarian cancer treatment or therapy; and
(c) comparing the level of the biomarker in the reference sample to the level of the biomarker in the subsequent sample,
   wherein an altered level of the biomarker in the subsequent sample compared to the reference sample is indicative of therapeutic response.

In an embodiment, the level of at least 2 biomarkers is determined and an altered level of at least 1 biomarker in the subsequent sample compared to the reference sample is indicative of therapeutic response. In another embodiment, the level of at least 3, 4, 5, 6, 7, 8, 9, 10 or more biomarkers is determined and an altered level of at least 1 biomarker in the subsequent sample compared to the reference sample is indicative of therapeutic response. In another embodiment, an altered level of at least 2, 3, 4, 5, 6, 7, 8, 9, 10 or more biomarkers in the subsequent sample compared to the reference sample is indicative of therapeutic response.

In an embodiment, the method for monitoring the therapeutic response of a subject with ovarian cancer comprising:
(a) determining a level of biomarker selected from the biomarkers set out in Table 2 in a subsequent sample of the subject, the subsequent sample taken subsequent to the subject receiving an ovarian cancer treatment or therapy; and
(b) comparing a reference level of the biomarker to the level of the biomarker in the subsequent sample,
   wherein an altered level of the biomarker in the subsequent sample compared to the reference level is indicative of therapeutic response.

In another embodiment, the level of biomarker in the subsequent sample is decreased compared to the reference sample, which is indicative of treatment efficacy or positive therapeutic response. In an embodiment, the level of biomarker is increased in the subsequent sample compared to the reference sample, which is indicative of treatment failure or negative therapeutic response.

In an embodiment, the altered level is a decrease in the level of the biomarker in the subsequent sample compared to the reference sample, and the decrease is indicative of treatment efficacy or positive therapeutic response. In another embodiment, the ratio of the level of the biomarker in the subsequent sample to the reference sample is less than 1, 0.8, 0.6, 0.4, 0.2, 0.1, 0.05, 0.001 or less. In another embodiment, the altered level is an increase in the level of the biomarker in the subsequent sample compared to the reference sample and the increase is indicative of treatment failure or negative therapeutic response. In another embodiment, the ratio of the level of the biomarker in the subsequent sample compared to the reference sample is greater than 1, 1.2, 1.5, 1.7, 2, 3, 3, 5, 10, 12, 15, 20 or more.

The ovarian cancer treatment or therapy can be any treatment that a skilled practitioner would administer or perform to treat ovarian cancer or a complication thereof. In addition the cancer treatment or therapy can include test therapies or clinical trial therapies. Accordingly, in an embodiment, the therapy is chemotherapy. In an embodiment the chemotherapy comprises carboplatin. In another embodiment, the chemotherapy comprises paclitaxel. In another embodiment, the therapy is surgery. In yet another embodiment, the therapy is a test therapy. In a further embodiment, the therapy is a combination therapy.

Monitoring therapeutic response is useful for example, for determining whether additional therapies should be considered by the skilled practitioner. Monitoring therapeutic response is also useful for determining efficacy of a test therapy, for example in a clinical trial.

A further aspect provides a method of prognosing survival in a subject with ovarian cancer comprising:
(a) determining a level of a biomarker in a test sample from the subject wherein the biomarker is selected from the biomarkers set out in Table 2; and
(b) comparing the level of the biomarker in the test sample with a control;
   wherein an altered level of the biomarker in the test sample compared to the control is indicative of the survival prognosis of the subject.

The method is useful at the time of diagnosis and/or after the subject has been treated. Accordingly, in an embodiment, the subject has not been treated. In another embodiment, the subject with ovarian cancer is treated with surgery and/or chemotherapy. In an embodiment the chemotherapy comprises carboplatin. In another embodiment, the chemotherapy comprises paclitaxel. In another embodiment, the therapy is surgery and chemotherapy.

In an embodiment, the level of at least 2 biomarkers is determined and an altered level of at least 1 biomarker in the test sample compared to the control indicative of the survival prognosis of the subject. In another embodiment, the level of at least 3, 4, 5, 6, 7, 8, 9, 10 or more biomarkers is determined and an altered level of at least 1 biomarker in the test sample compared to the control indicative of the survival prognosis of the subject. In another embodiment, an altered level of at least 2, 3, 4, 5, 6, 7, 8, 9, 10 or more biomarkers in the test sample compared to the control indicative of the survival prognosis of the subject.

The altered level can be an increased level and/or a decreased level. For example, the control corresponds to a biomarker level in a control subject or population of control subjects known to not have ovarian cancer, such that detection of an increased level of the biomarker in the sample compared to the control is indicative of poor survival. The positive control corresponds to a biomarker level in a positive control subject or population of positive control subjects known to have ovarian cancer, detection of a decreased level of the biomarker in the sample compared to the positive control is indicative of good survival. Similarly, where the control corresponds to a threshold level, below which is indicative of good survival and above which is indicative of poor survival, a decrease in the level of the biomarker in the sample compared to the control, is indicative that the subject has a good survival prognosis and an increase in the level of the biomarker in the sample compared to the control, is indicative that the subject has a poor survival prognosis.

In an embodiment, the control is a sample of, or a numerical value that corresponds to, a control subject or subjects without ovarian cancer. In an embodiment, the altered level is an increased level, wherein an increased level is indicative of poor survival. In an embodiment, the ratio of the level of the biomarker in the test sample compared to the control is greater than 1, 1.2, 1.5, 1.7, 2, 3, 3, 5, 10, 12, 15, 20 or more. In another embodiment, the positive control is a sample of, or a numerical value that corresponds to, a subject or subjects with ovarian cancer, for example late stage ovarian cancer. In another embodiment, the altered level is a decreased level, wherein the decreased level is indicative of good survival. In an embodiment, the ratio of the level of the biomarker in the test sample compared to the positive control is less than 1, 0.8, 0.6, 0.4, 0.2, 0.1, 0.05, 0.001 or less.

The control can be any suitable sample, biomarker level and/or corresponding numerical value to which the subject's sample can be compared. For example, the control can be a sample derived from a subject without ovarian cancer. Alternatively, a positive control can be a sample derived from a subject with ovarian cancer. Accordingly in an embodiment, the control is a sample of a subject without ovarian cancer. In another embodiment, the control is a numerical value that corresponds to a subject without ovarian cancer. In another embodiment, the positive control is a sample derived from a subject with ovarian cancer. In a further embodiment, the positive control is a numerical value that corresponds to a subject with ovarian cancer.

The control or positive control can also comprise a sample taken from the subject that is suitable for comparison. In an embodiment, the positive control or reference sample is a sample previously taken from the subject with ovarian cancer. In an embodiment, the reference sample is a sample taken prior to receiving a therapy, for example before receiving chemotherapy and/or surgery.

Further, more than one control and/or positive control may be utilized. For example, the level of the biomarker in the test sample can be compared to positive controls or positive control levels that associate with disease severity, to further stratify the subject. For example, one control can correspond to a biomarker level in a control subject or populations of control subjects known to not have ovarian cancer, an additional positive control can correspond to a biomarker level in a positive control subject or populations of positive control subjects known to have late stage ovarian cancer, and/or an additional positive control can correspond to a biomarker level in a positive control subject or populations of positive control subjects known to have early stage ovarian cancer. An increased level in the test sample compared to the control corresponding to the control known to not have ovarian cancer, and a similar level of the biomarker in the test sample compared to the early stage ovarian cancer positive control is indicative of early stage ovarian cancer. Similarly, an increased level in the test sample compared to the control corresponding to the control known to not have ovarian cancer and a similar biomarker level in the test sample compared to the late stage ovarian cancer positive control is indicative of late stage ovarian cancer. Similarly, more than one control and/or positive control can be used to prognose survival. For example two or more positive controls corresponding to biomarker levels in subjects treated with a particular therapy and/or having shown a particular survival trajectory, can be used.

Accordingly, in an embodiment, an increased level of the biomarker in the sample compared to a control, and a similar or increased level of the biomarker compared to a positive control, wherein the positive control corresponds to a biomarker level in early stage ovarian cancer, is indicative of early stage ovarian cancer. In another embodiment, an increased level of the biomarker in the sample compared to the control and a similar or increased level of the biomarker compared to a positive control, wherein the positive control corresponds to a biomarker level in late stage ovarian cancer, is indicative of late stage ovarian cancer.

Another aspect provides, a method of detecting relapse of ovarian cancer in a subject previously having ovarian cancer comprising:
(a) determining a level of a biomarker in a test sample from the subject, wherein the biomarker is selected from the biomarkers set out in Table 2; and
(b) comparing the level of the biomarker in the test sample with a control and/or positive control;
   wherein detecting an altered level of the biomarker in the test sample compared to the control or positive control is indicative of relapse of ovarian cancer in the subject.

In an embodiment, the biomarker is selected from AGRN Agrin precursor, BCAM Lutheran blood group glycoprotein precursor, C14orf141, CD248 Isoform 1 of Endosialin precursor, CD59 CD59 glycoprotein precursor, CLU Clusterin precursor, COMP 80 kDA protein, CPA4 Carboxypeptidase A4 precursor, CST3 Cystatin-C precursor, CST6 Cystatin-M precursor, CTGF Isoform 1 of Connective tissue growth factor precursor, DAG1Dystroglycan precursor, DKK3 Dickkopf-related protein 3 precursor, DSC2 Isoform 2A of Desmocillin-2 precursor, DSG2 desmoglein 2 preproprotein, ECM1 Extracellular matrix protein 1 precursor, EFEMP1 Isoform 1 of EGF-containing fibulin-like extracellular matrix protein 1 precursor, FAM3C Protein FAM3C precursor, FBLN1 Isoform C of Fibulin-1 precursor, FOLR1 Folate receptor alpha precursor, FSTL1 Follistatin-related protein 1 precursor, GLOD4 Uncharacterized protein of C17orf25, and GM2A Ganglioside GM2 activator precursor, GPX3 Glutathine peroxidase 3 precursor, HSPG2 Basement membrane-specific heparan sulfate proteoglycan core protein, HTRA1 Serine protease HTRA1 precursor, IGFBP2 Insulin-like growth factor-binding 2 precursor, IGFBP3 Insulin-like growth factor-binding 3 precursor, IGFBP4 Insulin-like growth factor-binding 4 precursor, IGFBP5 Insulin-like growth factor-binding 5 precursor, IGFBP6 Insulin-like growth factor-binding 6 precursor, IGFBP7 Insulin-like growth factor-binding 7 precursor, LRG1 Leucine-rich alpha-2-glycoprotein precursor, MST1 Hepatocyte growth factor-like protein precursor, MXRA5 matrix-remodelling-associated protein 5 precursor, NID2 Nidogen-2 precursor, NPC2 Epididymal secretory protein E1 precuror, NUCB1 Nucleobindin-1 precursor, PCOLCE Procollagen C-endopeptidase enhancer 1 precursor and documented synonyms of the above proteins. The amino acid and nucleic sequences of the biomarkers of the disclosure are known and can be identified by searching the polypeptide IPI accession numbers which are provided in Table 5 and are herein incorporated by reference.

In an embodiment, the biomarker selected from Table 2 is nidogen-2 (also interchangeably referred to as nidogen-2 precursor or NID2) In an embodiment, the level of nidogen-2 is determined for screening for, diagnosing, or detecting ovarian cancer or an increased likelihood or developing ovarian cancer in a subject. In a further embodiment, the level of nidogen-2 is determined for monitoring the therapeutic response of a subject with ovarian cancer. In a further embodiment, the level of nidogen-2 is determined for prognosing survival in a subject with ovarian cancer. In yet another embodiment, the level of nidogen-2 is determined for detecting relapse in a subject previously having ovarian cancer. In certain embodiments, the ovarian cancer is late stage ovarian cancer. In other embodiments, the ovarian cancer is stage 2 or stage 3 ovarian cancer. In still other embodiments, the ovarian cancer is serous ovarian cancer or serous cystadenocarcinoma ovarian cancer.

In an embodiment, the level of nidogen-2 associated with ovarian cancer or an increased likelihood of developing ovarian cancer is greater than 20 micrograms/L, greater than 25 micrograms/L, greater than 26 micrograms/L, greater than 27 micrograms/L, greater than 28 micrograms/L, greater than 29 micrograms/L and/or greater than 30 micrograms/L. In another embodiment the level of nidogen-2 in the test sample associated with ovarian cancer or an increased likelihood of developing ovarian cancer is greater than 20-25 micrograms/L, and/or greater than 25-30 micrograms/L, for example as measured for a serum sample by an ELISA assay. In an embodiment, the level of nidogen-2 associated with poor prognosis is greater than 20 micrograms/L, greater than 25 micrograms/L, greater than 26 micrograms/L, greater than 27 micrograms/L, greater than 28 micrograms/L, greater than 29 micrograms/L and/or greater than 30 micrograms/L. In another embodiment the level of nidogen-2 in the test sample associated with poor prognosis is greater than 20-25 micrograms/L, and/or greater than 25-30 micrograms/L. In a further embodiment, the level of nidogen-2 associated with relapse in a subject is greater than 20 micrograms/L, greater than 25 micrograms/L, greater than 26 micrograms/L, greater than 27 micrograms/L, greater than 28 micrograms/L, greater than 29 micrograms/L and/or greater than 30 micrograms/L. In another embodiment the level of nidogen-2 in the test sample associated with relapse is greater than 20-25 micrograms/L, and/or greater than 25-30 micrograms/L. As a person skilled in the art would understand, the level associated with ovarian cancer, poor prognosis and/or relapse will vary with for example, the sample type, the detection method used and sample processing such as dilution.

The level of biomarker determined can comprise level of expression of a biomarker, for example the level of polypeptide or RNA expressed; the level of a soluble biomarker, and/or the level of secretion, release, cleavage or shedding, for example the secretion, release or shedding of a biomarker polypeptide or fragment, such as an extracellular fragment; and/or the level of post-translational modification, for example glycosylation, and/or phosphorylation of the biomarker compared to a control and/or previously taken or reference sample. For example, the level of glycosylation can vary in subjects with or without ovarian cancer. For example a biomarker can be glycosylated in subjects with ovarian cancer and not glycosylated in subjects without ovarian cancer. Alternatively, a biomarker can be glycosylated in subjects without ovarian cancer and not glycosylated in subjects with ovarian cancer. The presence or absence of glycosylation may be specific to a certain type of glycosylation e.g. N-linked or O-linked glycosylation, and/or specific to a specific residue or group of residues eg. N-terminus of protein comprises N-linked glycosylation in subjects with ovarian cancer. For example, hexosamine analysis of nidogen-2 demonstrates 25±2 glucosamine and 19±2 galactosamine residues. Nidogen-2 contains 5 predicted N-glycosylation sites (Asn at position 417, 658, 693, 703 and 1124), two tyrosine residues (position 310 and 317) located in a consensus region for O-sulfation and a substantial number of O-glycosylation sites. (Reference Kohfeldt et al., 1998, J Mol Biol, 99-109).

A number of polypeptide fragments were detected as described in the Examples below. The polypeptide fragments, can result for example from increased shedding or cleavage of extracellular portions of biomarker polypeptides. The polypeptide fragments can also result for example from cleavage or degradation of secreted polypeptides. The level of the biomarker determined is optionally detected in a complex, e.g. homo or heterodimer or higher order complex, as in a microparticle. The biomarker is also optionally uncomplexed e.g. free in the sample.

In an embodiment, the level of biomarker determined is the level of polypeptide product of the biomarker. In an embodiment, the level of biomarker determined is the level of a fragment of a polypeptide product of the biomarker. In an embodiment, the polypeptide product or fragment thereof is increased in the test sample compared to the control. In another embodiment, the polypeptide product or fragment thereof is increased in the subsequent sample compared to the reference sample. In an embodiment, the polypeptide product or fragment thereof is decreased in the test sample compared to control. In another embodiment, the polypeptide product or fragment thereof is decreased in the subsequent sample compared to the reference sample.

The test sample can be any biological fluid, cell or tissue sample from a subject, which can be assayed for biomarker levels. Similarly, the control or control sample or positive control or positive control sample can be any biological fluid, cell or tissue sample from a subject, which can be assayed for biomarker levels. In an embodiment, the test sample, control and/or positive control comprises a biological fluid. In an embodiment, the test sample, control and/or positive control comprises blood, serum, plasma, tumour cells, tissue specimen, ascites, ascitic fluid, sputum, urine, pleural effusions, tear drops and/or bodily secretions. In an embodiment, the test sample, control and/or positive control comprises serum. In another embodiment, the test sample, control and/or positive control comprises ascites. In a further embodiment, the test sample, control and/or positive control comprises the fluid fraction of ascites. In a further embodiment, the test sample, control and/or positive control comprises a cell fraction of ascites.

In certain embodiments, the test sample and the control and/or positive control sample are the same sample type or fraction. In an embodiment, the test sample and the control and/or positive control sample each comprise a biological fluid. In another embodiment, embodiment, the test sample and the control and/or positive control sample each comprise ascites and/or acites fluid. In an embodiment, the test sample and the control and/or positive control sample each comprise blood, plasma and/or serum. In another embodiment, the test sample and positive control sample each comprise tumor cells. In yet a further embodiment, the test sample and control and/or positive control sample comprises tissue specimen, pleural effusions, and/or tear drops.

In other embodiments, the test sample and the control and/or positive control sample are handled similarly. For example, preparation and similar handling of samples can be very important for determining accurate levels for comparison. For example, a test and a control sample may both be stored frozen, for example at −20 C, −80 C and/or in liquid nitrogen. They may also be similarly manipulated and/or fractionated. In an embodiment, the samples, such as the test and control and/or positive control samples, are defrosted similarly and kept at room temperature for the same amount of time to prevent and/or limit protein degradation. Samples are in an embodiment, diluted using the same stock buffer/diluents.

The levels of additional biomarkers can also be determined with each of the methods described herein. For example, biomarkers are known in the art to be associated with ovarian cancer. In an embodiment, the methods described herein further comprise detecting an additional biomarker. In an embodiment, the biomarker is selected from the additional biomarkers set out in Table 1.

A number of methods can be used to determine a level of a biomarker. The level of a biomarker can be determined for example using a detection agent, wherein the detection agent specifically binds the biomarker, e.g. for example forms an antibody antigen complex, and permits quantitation of the biomarker, e.g. for example through a detectable label.

In an embodiment, the detection agent comprises an antibody or antibody fragment that binds a biomarker. In another embodiment, the detection agent comprises an isolated antibody or isolated antibody fragment.

Antibodies having specificity for a specific polypeptide, such as a polypeptide product of a biomarker described of the disclosure, may be prepared by conventional methods. A mammal, (e.g. a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be obtained and, if desired, polyclonal antibodies isolated from the sera.

To produce monoclonal antibodies, antibody-producing cells (lymphocytes) can be harvested from an immunized animal and fused with myeloma cells by standard somatic cell fusion procedures thus immortalizing these cells and yielding hybridoma cells. Such techniques are well known in the art, (e.g. the hybridoma technique originally developed by Kohler and Milstein (Nature 256:495-497 (1975)) as well as other techniques such as the human B-cell hybridoma technique (Kozbor et al., Immunol. Today 4:72 (1983)), the EBV-hybridoma technique to produce human monoclonal antibodies (Cole et al., Methods Enzymol, 121:140-67 (1986)), and screening of combinatorial antibody libraries (Huse et al., Science 246:1275 (1989)). Hybridoma cells can be screened immunochemically for production of antibodies specifically reactive with the peptide and the monoclonal antibodies can be isolated.

In an embodiment, the detection agent, including isolated polypeptides or antibodies, is labeled with a detectable marker. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

In another embodiment, the detectable signal is detectable indirectly. For example, a secondary antibody that is specific for a biomarker described herein and contains a detectable label can be used to detect the biomarker.

The disclosure also contemplates the use of “peptide mimetics” for detecting biomarkers of the disclosure. Peptide mimetics are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of detection agents specific for polypeptide products of the biomarkers of the disclosure. Peptide mimetics also include peptoids, oligopeptoids (Simon et al (1972) Proc. Natl. Acad, Sci USA 89:9367).

A person skilled in the art will appreciate that a number of quantitative proteomic methodologies can be used to determine the amount of the biomarker polypeptide, including immunoassays such as Western blots, ELISA, and immunoprecipitation followed by SDS-PAGE immunocytochemistry.

Accordingly, an aspect relates to using an immunoassay, for example an immunoassay described herein, for detecting biomarker polypeptide products. In an embodiment, an immunoassay is used for screening for, detecting or diagnosing ovarian cancer or the likelihood of developing ovarian cancer in a subject, monitoring the therapeutic response of a subject to an ovarian cancer treatment, and/or prognosing survival. In an embodiment, the immunoassay used comprises an antibody immobilized to a solid support and a detection antibody. In another embodiment, the detection antibody is a biotinylated antibody. In another embodiment, the immunoassay used is an enzyme-linked immunosorbant assay (ELISA). In a further embodiment, the ELISA is a direct or indirect sandwich type ELISA. In a further embodiment, the immunoassay used is a diffraction immunoassay.

Multiplex analysis can be utilized, detecting levels of internal controls and/or multiple biomarker levels.

The level of a biomarker within for example, a biological fluid can also be assessed by mass spectroscopy based technologies. For example, biomarker peptides can be quantified using iTRAQ™, SILAC, Tandem Mass Tag (TMT) and other similar labeling reagents in conjunction with mass spectrometry.

In addition to measuring the level of polypeptide products of biomarkers of the disclosure, differential expression of the RNA products of the biomarkers described herein can be used to screen for, detect or diagnose ovarian cancer or a likelihood of developing ovarian cancer. In an embodiment, the method of screening for, diagnosing or detecting ovarian cancer comprises using detection agents comprising isolated nucleic acid sequences that hybridize to a RNA product of a biomarker set out in Table 2.

Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41(% (G+C)−600/1), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In some embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm−5° C. based on the above equation, followed by a wash of 0.2×SSC/0.1% SDS at 60° C. In an embodiment moderately stringent hybridization conditions are employed. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 1989, 6.3.1-6.3.6 and in: Sambrook et al., Molecular Cloning, a Laboratory Manual, Cold Spring Harbor Laboratory Press, 1989, Vol. 3.

The stringency may be selected based on the conditions used in the wash step. By way of example, the salt concentration in the wash step can be selected from a high stringency of about 0.2×SSC at 50° C. In addition, the temperature in the wash step can be at high stringency conditions, at about 65° C. Accordingly in certain embodiments, high stringency conditions are employed.

A person skilled in the art will appreciate that a number of methods can be used to measure or detect the level of RNA products of the biomarkers of the disclosure within a sample, including microarrays, RT-PCR (including quantitative RT-PCR), nuclease protection assays and northern blots.

In an embodiment, the detection agent is a probe. In another embodiment, the detection agent is a primer. In a further embodiment, the detection agent is a set of primers, “biomarker specific primers”, which are useful for producing cDNA.

It is contemplated that the methods described herein can be used in combination with other methods of screening for, diagnosing or detecting ovarian cancer.

### b) Methods of Identifying Biomarkers and Serum/Ascites Proteomes

Another aspect relates to methods of identifying an ascites proteome or subproteome and to methods of identifying an ovarian cancer biomarker. Since ascites fluid contains many cells of tumor origin, in addition to other soluble growth factors that have been associated with invasion and metastasis^{11, 12}, this fluid contains the secretome of ovarian cancer cells while reflecting other microenvironmental factors of the malignancy. Thus, applying the ever-advancing technique of proteomic analysis on ascites, it may be possible to discover novel biomarkers that are more sensitive and specific than those currently available.

As biomarkers may be present at low concentrations¹⁸, and ascites, like serum, contains many high-abundance proteins (with a protein concentration range spanning at least nine orders of magnitude¹⁹), extensive sample fractionation is necessary if biomarkers are to be found successfully using mass spectrometry.

An in-depth subproteome analysis of ascites fluid based on multiple separation and fractionation techniques, followed by mass spectrometry analysis is described in the Examples. Attempts at identifying an ascites proteome have been made. While Kislinger's group previously identified over 2500 proteins within ascites¹⁷, only 229 proteins were identified in the fluid fraction. Described herein is the most extensive ascites fluid subproteome consisting of 445 unique proteins, many of which overlap with previous data¹⁷ including proteins proposed as candidate serological ovarian cancer biomarkers. After applying multiple data mining criteria to the list of proteins, a group of 52 proteins was assembled which represent good candidates as ovarian cancer biomarkers.

Accordingly, an embodiment provides a method of identifying an ascites or serum proteome or subproteome in a subject with ovarian cancer comprising:
(a) obtaining a test sample from the subject, wherein the test sample comprises ascites and/or ascites fluid and/or serum;
(b) gel filtering and/or centrifugal ultrafiltering the test sample;
(c) selecting fractions depleted of high molecular weight proteins;
(d) concentrating and digesting the fractions; and
(e) analyzing the digested fractions by mass spectrometry to identify the proteins in the fractions;

wherein the collection of unique proteins identifies the ascites or serum proteome of the subject with ovarian cancer.

Another embodiment provides a method of identifying an ovarian cancer biomarker in a subject with ovarian cancer comprising:
(a) obtaining a test sample from the subject, wherein the test sample comprises ascites and/or ascitic fluid and/or serum;
(b) gel filtering and/or centrifugal ultrafiltering the test sample;
(c) selecting fractions depleted of high molecular weight proteins;
(d) concentrating and digesting the fractions;
(e) analyzing the digested fractions by mass spectrometry to identify a plurality of unique polypeptides in the fractions;
(f) selecting one or more polypeptide(s) of the plurality of unique polypeptides as a candidate biomarker for validation; and
(g) validating an association of a polypeptide level of the collection of unique polypeptides with ovarian cancer or an increased likelihood of ovarian cancer;

wherein the association with ovarian cancer or the increased likelihood of ovarian cancer is indicative that the polypeptide is a biomarker associated with ovarian cancer.

In an embodiment, the gel filtering step comprises size exclusion chromatography. In an embodiment, the biomarker is detectable in serum. In another embodiment, the biomarker is detectable in ascites/ascitic fluid.

In an embodiment, the selection step (f) comprises:
I. applying filtering criteria selected from eliminating intracellular polypeptides; eliminating high abundance serum proteins; eliminating known ovarian cancer biomarkers; eliminating polypeptides found in only one fractionation protocol; and eliminating polypeptides identified as intracellular polypeptides in ovarian cancer cell lines; and
II. selecting a polypeptide remaining in the collection after the application of the filtering criteria.

In an embodiment, the subject has late stage ovarian cancer. In another embodiment, the subject has early stage ovarian cancer.

### III. COMPOSITIONS

In another aspect, the disclosure relates to a composition comprising a collection of two or more detection agents, wherein at least one detection agent detects a biomarker selected from the biomarkers set out in Table 2, and the second detection agent optionally detects a biomarker listed in Table 2 or an additional biomarker for example, a biomarker listed in Table 1 or CA125, wherein the composition is used to measure the level of at least two of the biomarkers. In an embodiment, the detection agent detects an extracellular portion of the biomarker. In an embodiment, at least one detection agent comprises a selective receptor molecule. In an embodiment, the detection agent detects Nidogen-2. In another embodiment, at least one detection agent comprises an antibody or an isolated antibody fragment.

### IV. IMMUNOASSAYS

Another aspect relates to an immunoassay for determining biomarker levels. An embodiment provides an immunoassay for detecting a biomarker comprising an antibody or antibody fragment immobilized on a solid support, wherein the antibody binds a biomarker wherein the biomarker is selected from the biomarkers set out in Table 2. In an embodiment, the biomarker is Nidogen-2. In an embodiment, immunoassay comprises an antibody immobilized to a solid support and a detection antibody. In an embodiment, the immunoassay is an ELISA. In another embodiment, the ELISA is an indirect sandwich type ELISA. In an embodiment, the immunoassay is a diffraction immunoassay. In a further embodiment, the ELISA is useful for determining a level of a biomarker in a method described herein.

### V. KITS

Also provided in an aspect of the disclosure is a kit. In an embodiment, the kit comprises at least two detection agents, wherein at least one of the detection agents detects a biomarker selected from the biomarkers set out in Table 2. The second detection agent optionally detects a biomarker listed in Table 2 or an additional biomarker for example a biomarker listed in Table 1 or CA125. In an embodiment, the kit optionally additionally includes instructions for use. The detection agents are used to measure the level of the two biomarkers. In an embodiment, the biomarker detected by at least one of the detection agents is nidogen-2. In another embodiment, the second detection agent detects CA-125. In an embodiment, at least one detection agent comprises a selective receptor molecule. In another embodiment, the selective receptor molecule comprises an isolated antibody or an isolated antibody fragment that specifically binds the selected biomarker.

The following non-limiting examples are illustrative of the disclosure:

### EXAMPLES

### Example 1

### Materials and Methods

Patients and Specimens: Ascites fluid was obtained with informed consent and IRB approved from women with advanced stage ovarian cancer undergoing paracentesis. These patients had stage IV serous ovarian carcinoma and they have been previously treated with surgery plus carboplatin/paclitaxel chemotherapy.
Sample Collection and Preparation: Ascites fluids were aliquoted in 1 mL portions and centrifuged at 16,000×g for 30 min at 4° C. three times, to separate the fluid from lipids and cellular components.
Gel Filtration: Gel filtration was performed using a 0.75×60 cm TSK-Gel G3000SW column (Tosoh Bioscience) attached to an Agilent 1100 HPLC system. The column was equilibrated with either (i) phosphate/sulfate buffer (10 mM NaH₂PO₄, 10 mM Na₂SO₄, pH 6.8) or (ii) 100 mM ammonium bicarbonate buffer, pH 7.8. Five hundred μL of ascites was loaded onto the system at a flow rate of 0.5 mL/min for 1 h. Forty successive injections were performed, collecting eluted fractions at 1 min intervals, starting at 20 minutes (column void volume). A total of 39 fractions, containing 20 mL each, were collected for each buffer type. Gel filtration experiments were performed in duplicate for each buffer. Each fraction was then analyzed for kallikrein 6 (KLK6) and total protein, followed by lyophilization to dryness.
Centrifugal Ultrafiltration: 15 mL of ascites were added to a pre-rinsed 50 KDa or 100 KDa nominal molecular weight limit cutoff Amicon Ultra-15 centrifugal filter device (Millipore). After 5 min of centrifugation at 4000×g in a swinging bucket rotor, unfiltered ascites was topped to 15 mL with water. This process was repeated until 15 mL of filtered ascites was obtained. The filtered ascites was then lyophilized to dryness and underwent trypsin digestion (see below). The 50 KDa-filtered ascites was analyzed directly by LC-MS/MS while the 100 KDa-filtered ascites underwent strong cation exchange liquid chromatography (see below) prior to LC-MS/MS analysis with a reverse-phase C18 column.
KLK6 ELISA Immunoassay: The concentration of kallikrein 6 (KLK6) in each eluted gel filtration fraction was measured by a sandwich-type immunoassay²⁰. In brief, a KLK6-specific monoclonal antibody (clone 27-4; developed in-house) was first immobilized in a 96-well white polystyrene plate by incubating 250 ng/100 μl/well in a coating buffer (50 mmol/L Tris, 0.05% sodium azide; pH 7.8) overnight. After washing three times with washing buffer (5 mmol/L Tris, 150 mmol/L NaCl, 0.05% Tween 20; pH 7.8), 50 μl of each pooled gel filtration fraction diluted 1:3 in 6% bovine serum albumin (BSA) or 50 μl of KLK6 standards were pipetted into each well, in addition to 50 μl of assay buffer (50 mmol/L Tris, 6% BSA, 0.01% goat IgG, 0.005% mouse IgG, 0.1% bovine IgG, 0.5 mol/L KCl, 0.05% sodium azide; pH 7.8) and incubated for 1 hour with shaking at room temperature. The plates were washed six times with the washing buffer, after which biotinylated detection antibody solution (100 μl; 15 ng anti-KLK6 (E24) monoclonal antibody in assay buffer) was added to each well and incubated for 1 hour at room temperature with shaking. The plates were then washed six times with the washing buffer. Subsequently, alkaline phosphatase-conjugated streptavidin solution (5 ng/well; Jackson ImmunoResearch, Westgrove, Pa.) in 6% BSA buffer (in 50 mmol/L Tris, 0.05% sodium azide; pH 7.8) were added to each well and incubated for 15 min with shaking, at room temperature. The plates were washed six times with the washing buffer and substrate buffer (100 μl; 0.1 mol/L Tris buffer; pH 9.1) containing 1 mmol/L of the substrate diflunisal phosphate, 0.1 mol/L NaCl, and 1 mmol/L MgCl₂ was added to each well and incubated for 10 min with shaking at room temperature. After adding 50 μl of developing solution containing Tb³⁺/EDTA complex, the fluorescence of each well was measured with the Wallac Envision 2103 multilabel reader. More details are given elsewhere²⁰.
Total Protein Assay: Total protein of each ascites fraction was quantified using a Coomassie (Bradford) protein assay reagent (Pierce). Five μl of each pooled gel filtration fraction and 5 μl of water were loaded in duplicate in a microtitre plate along with the reagent, and protein concentrations were estimated by reference to absorbance obtained for a series of bovine albumin standard protein dilutions.
Trypsin Digestion: Each lyophilized sample was denatured using 8 M urea, reduced with dithiothreitol (DTT) (final concentration, 13 mM; Sigma) at 50° C. followed by alkylation with 500 mM iodoacetamide (Sigma) with shaking at room temperature in the dark. The samples were then desalted using a NAP5 column (GE Healthcare). Samples were lyophilized and resuspended in trypsin buffer (1:50 ratio of trypsin (Promega, sequencing grade modified porcine trypsin):protein concentration; 120 μl of 50 mM ammonium bicarbonate, 100 μl of methanol, 150 μl of water) overnight in a 37° C. waterbath and then lyophilized to dryness.
Strong Cation Exchange Liquid Chromatography: Trypsin-digested lyophilized sample was reconstituted in 120 μl of mobile phase A (0.26 M formic acid in 10% acetonitrile). The samples were directly loaded onto a PolySULFOETHYL A™ column (The Nest Group, Inc.) containing a hydrophilic, anionic polymer (poly-2-sulfoethyl aspartamide) with a pore size of 200-Å and a diameter of 5 μm. A 1-h fractionation run was performed using high-performance liquid chromatography (HPLC), with an Agilent 1100 system at a flow rate of 200 μl/min. A linear gradient of mobile phase B (0.26 M formic acid in 10% acetonitrile and 1 M ammonium formate) was added as the elution buffer. The eluate was monitored at a wavelength of 280 nm. Forty fractions, 200 μl each, were collected every minute after the start of the elution gradient. These forty fractions were pooled into eight combined fractions (each pool consisting of five fractions) and concentrated to ˜200 μl using a SpeedVac system preceding mass spectrometry analysis. Prior to each run, a protein cation exchange standard (Bio-Rad) was applied to evaluate column performance.
Mass Spectrometry: The samples from each pooled fraction of each individual separation experiment were desalted using a ZipTip C₁₈ pipette tip (Millipore) and eluted in 4 μl of Buffer B (90% acetonitrile, 0.1% formic acid, 10% water, 0.02% trifluoroacetic acid). Eighty μl of Buffer A (95% water, 0.1% formic acid, 5% acetonitrile, 0.02% trifluoroacetic acid) were added to each sample and 40 μl were loaded on an Agilent 1100 HPLC system by the autosampler and injected onto a 2 cm C₁₈ trap column (inner diameter, 200 μm). Peptides were eluted from the trap column onto a resolving-5-cm analytical C₁₈ column (inner diameter, 75 μm) with an 8-μm tip (New Objective). This liquid chromatography setup was coupled online to a two-dimensional linear ion trap (LTQ, Thermo Inc.) mass spectrometer using a nanoelectrospray ionization source (ESI) in data-dependent mode. Each fraction underwent a 120-min gradient and eluted peptides were subjected to tandem mass spectrometry (MS/MS). Data files (DTAs) were created using the Mascot Daemon (version 2.2) and extract_msn. The parameters for DTA creation were: minimum mass, 300 Da; maximum mass, 4000 Da; automatic precursor charge selection; minimum peaks, 10 per MS/MS scan for acquisition; and minimum scans per group, 1.
Data Analysis: The resulting mass spectra from each fraction were analyzed using Mascot (Matrix Science, London, UK; version 2.2) and X!Tandem (Global Proteome Machine Manager, version Jan. 6, 2006) search engines on the non-redundant International Protein Index (IPI) human database (version 3.27) which included the forward and reversed sequences for calculating false positive error of each protein. Up to one missed cleavage was allowed, and searches were performed with fixed carbamidomethylation of cysteines and variable oxidation of methionine residues. A fragment tolerance of 0.4 Da and a parent tolerance of 3.0 Da were used for both search engines with trypsin as the digestion enzyme. The resulting files were all loaded into Scaffold (version 2.0, Proteome Software Inc., Portland, Oreg.) which validated each MS/MS-based peptide and protein identification. Peptide identifications were accepted if they could be established at greater than 95% probability as specified by the PeptideProphet algorithm²¹. Protein identifications were accepted if they could be established at greater than 95% probability and contained at least one identified peptide. Protein probabilities were assigned by the ProteinProphet algorithm²². The DAT and XML files for each fraction were inputted into Scaffold to cross-validate Mascot and X!Tandem data files. All biological samples were searched with MudPIT (multidimensional protein identification technology) option clicked. Sample reports were exported from Scaffold and each protein identification was assigned a cellular localization based on information available from Swiss-Prot, Genome Ontology (GO), Euk-mPLoc²³ and other publicly available databases.

### Results

Complex biological fluids such as serum and ascites fluid contain thousands of proteins with a concentration range spanning at least nine orders of magnitude¹⁹. The major challenge preventing in-depth analysis of these proteomes by mass spectrometry is the presence of abundant proteins such as albumin and immunoglobulins, which make up 65-97% of serum proteins. These abundant proteins limit the ionization efficiency during mass spectrometric analysis, preventing the identification of low abundance proteins. While various techniques have been used for albumin and immunoglobulin depletion^{24, 25}, size exclusion chromatography and centrifugal ultrafiltration to fractionate ascites fluid on the basis of molecular mass was selected. Since the top 20 most abundant serum proteins have molecular masses greater than 30 KDa, 30 KDa was chosen as the approximate molecular mass cutoff for the identification of the ascites fluid subproteome.

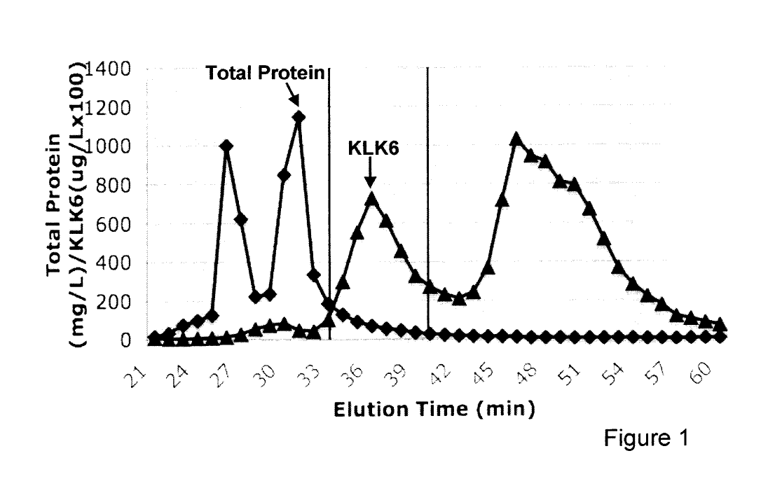

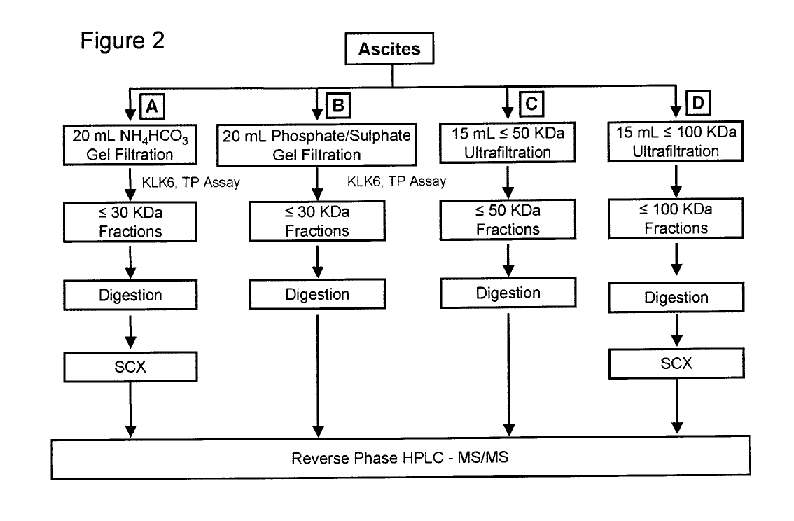

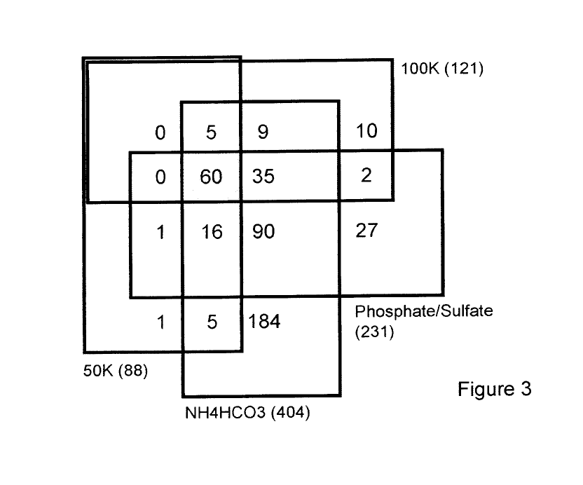

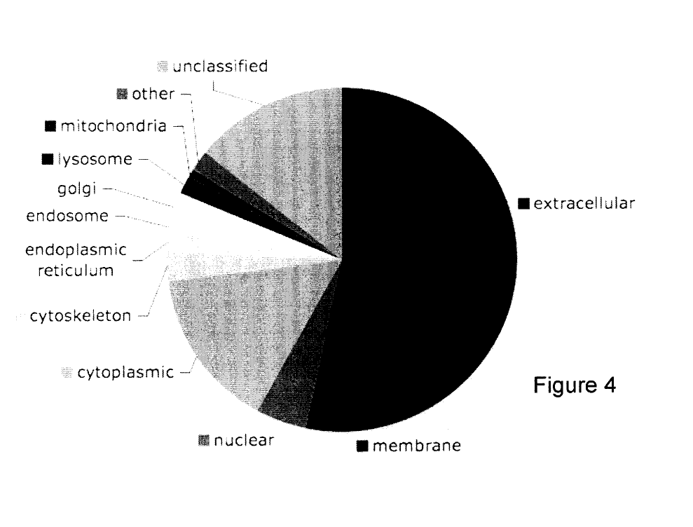

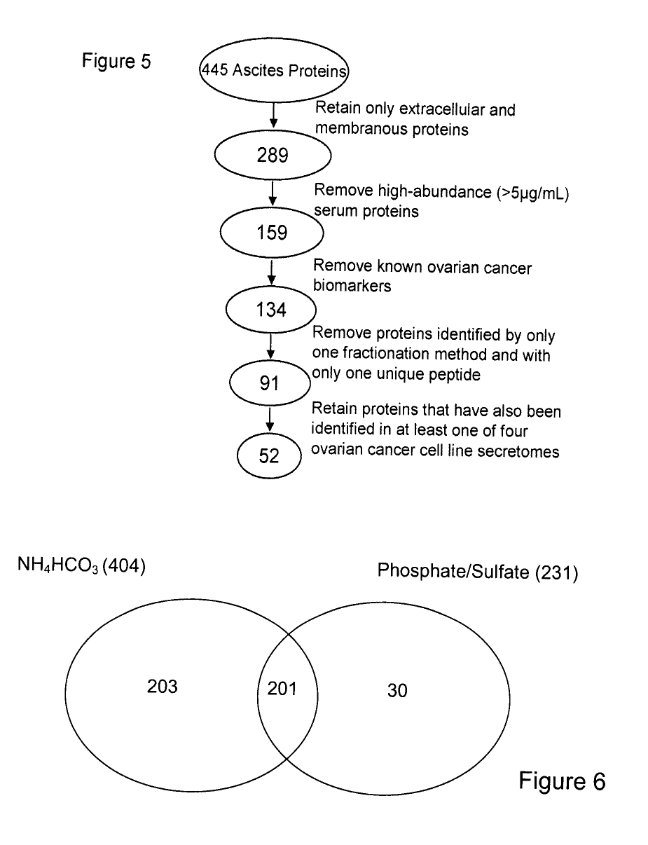

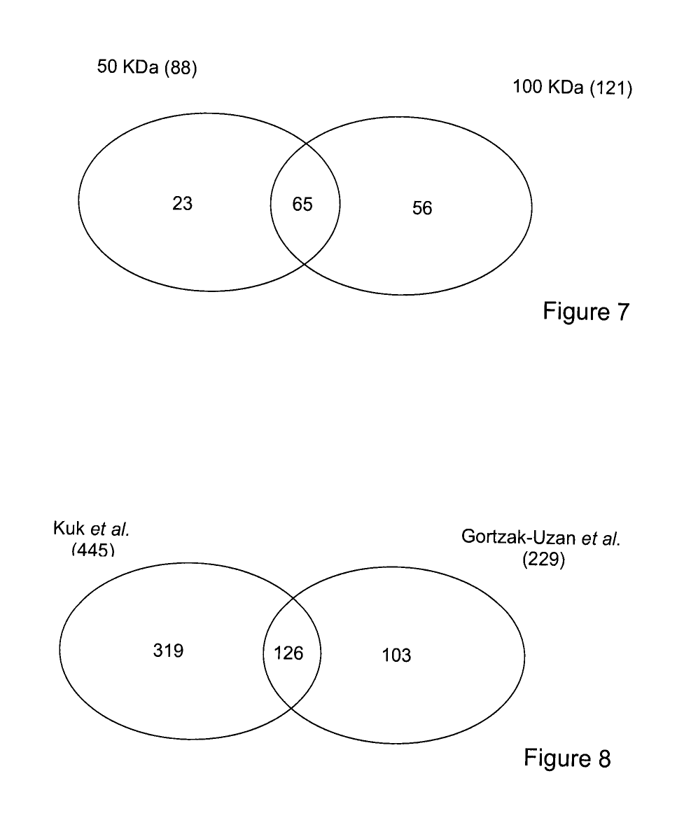

Identification of Proteins by Mass Spectrometry—Gel Filtration: 20 mL of ascites fluid from one patient with disseminated ovarian cancer were used and size exclusion chromatography was performed in duplicate using two different mobile phase buffer solutions; phosphate/sulfate and ammonium bicarbonate. After performing KLK6 enzyme-linked immunosorbent assay (ELISA) and total protein assay on the eluate, fractions containing KLK6 (˜30 KDa) and lower molecular mass proteins were selected for further fractionation or trypsin digestion and mass spectrometry (FIGS. 1 and 2). While with this method the majority of albumin and immunoglobulins were removed, some early fractions still contained a significant amount of total protein (FIG. 1). Hence, 10 fractions starting from the KLK6 peak were collected and refractionated with gel filtration to remove additional amounts of high-abundance proteins. Four hundred and four proteins were identified with the ammonium bicarbonate buffer and 231 proteins were identified using the phosphate/sulfate buffer system (duplicate analysis with both systems). There was a 46% overlap between the two buffer systems; when data were combined, a total of 434 unique proteins were identified (FIG. 6). Only 30 additional proteins were identified with the phosphate/sulfate buffer system.
Identification of Proteins by Mass Spectrometry—Ultrafiltration: 15 mL of ascites from two different patients with late stage ovarian cancer underwent ultrafiltration using Millipore centrifugal ultrafiltration devices with a nominal molecular mass cutoff of 50 KDa and 100 KDa. These cutoffs were chosen based on the guidelines provided by the manufacturer regarding yields of proteins in the eluates. To obtain a good yield of filtrated proteins with molecular mass of ≦30 KDa, it was suggested to use ultrafiltration devices with a nominal molecular mass cutoff two to four times higher than the desired protein mass. Eighty eight and 121 proteins were identified from the 50 KDa and 100 KDa filtrates, respectively. There was a 45% overlap between the 50 KDa and 100 KDa filtrates; when data were combined, a total of 144 unique proteins were identified (FIG. 7). When all data were combined (gel filtration and ultrafiltration) the number of unique proteins identified was 445 (FIG. 3).
A complete list of proteins identified, along with their number of unique peptides in each experiment was composed. Redundancies and false-positive proteins were removed from the list. Detailed information on all proteins identified for each experiment, including number of unique peptides identified per protein, peptide sequences, precursor ion mass, and charge states was determined. A total of 445 unique proteins were identified from all 6 individual experiments; 215 more proteins were identified in the soluble ascites fraction than the previously published proteome of ascites fluid by Gortzak-Uzan et al.¹⁷. The false-positive rate was 2.4%.
Cellular Localization of Identified Proteins: Each unique protein identified was classified according to its cellular localization based on information available from Swiss-Prot, Gene Ontology and other publicly available databases. FIG. 4 shows the cellular distribution of the 445 proteins with known localizations. When one protein is localized in more than one cellular compartment, all of the categories were accounted for, non-exclusively. This resulted in a total percentage greater than 100%. Of the proteins, 14% were not classified as none of the sources was informative. The majority of the classified proteins were extracellular (40%) and membrane-bound (12%). One hundred and fifty seven of the 445 identified ascites fluid proteins were also identified in the Plasma Proteome Database (Table 3). This does not mean that the remaining 288 proteins are exclusive to ascites fluid as the plasma proteome is incomplete and still requires more in-depth analysis. The data suggest that many of the proteins identified are secreted by the tumor cells or the tumor microenvironment.
Identification of Candidate Biomarkers: To identify biomarker candidates, a set of filtering criteria to the list of unique proteins was applied (FIG. 5).
1. Proteins which are not extracellular or membranous were removed: From the list of 445 unique proteins, 148 proteins were eliminated, resulting in a shortened list of 289 extracellular and membranous proteins (Table 3). Extracellular and membranous proteins were selected, as these proteins have the highest potential of being found in the circulation and hence can be detected by non-invasive serum-based tests.
2. All known high-abundance serum proteins (concentration >5 μg/mL) such as albumin, immunoglobulins and complement-related proteins were removed: Of the 289 extracellular and membranous proteins, 130 were classified as high abundance serum proteins and were removed, leaving a list of 159 proteins.
3. Proteins previously studied in the serum of ovarian cancer patients were removed: The 159 remaining proteins were individually searched in Pubmed. Twenty-five of these proteins were examined in the past as ovarian cancer biomarkers (Table 1). Five of those belong to the kallikrein family of biomarkers.
4. Proteins found in only one fractionation protocol and with a single unique peptide were removed: Forty three proteins were removed, with 91 proteins remaining for further selection.
5. Analysis of the proteome and secretome (secreted and membrane-bound proteins) of four ovarian cancer cell lines (HTB75, serous; TOV112D, endometrioid; TOV21G, clear cell; RMUG-S, mucinous) identified a total of 1689 proteins. 154 proteins overlapped with the list of 445 ascites proteins while seventy-three of these proteins overlapped with the list of 289 extracellular and membranous ascites fluid proteins (Table 4). The results of the cell line analysis have been used to further confirm the proteins identified within the ascites samples. Of the 91 remaining proteins, 52 proteins were identified as extracellular or membranous proteins in the supernatant of at least one of the four ovarian cancer cell lines studied. These remaining 52 proteins, which passed all of the selection criteria, represent the panel of candidate ovarian cancer biomarkers (Table 2).

TABLE 1
Identified proteins previously examined as ovarian cancer biomarkers

| | Molecular | Refer- |
|---|---|---|
| Protein Name¹ | Mass, Da | ences |
| AFM Afamin precursor | 69,052 | ³⁶ |
| CHI3L1 Chitinase-3-like protein 1 precursor | 42,609 | ³⁷ |
| (YKL-40) | | |
| CLEC3B Hypothetical protein DKFZp686H17246 | 17,776 | ^{38, 39} |
| (tetranectin) | | |
| KLK10 Kallikrein-10 precursor | 30,120 | ^{40, 41} |
| KLK11 Isoform 1 of Kallikrein-11 precursor | 27,448 | ⁴² |
| KLK6 Kallikrein-6 precursor | 26,838 | ⁴³ |
| KLK7 Isoform 1 of Kallikrein-7 precursor | 27,507 | ⁴⁴ |
| KLK9; KLK8 Isoform 1 of Neuropsin precursor | 28,029 | ⁴⁵ |
| LCN2 Neutrophil gelatinase-associated lipocalin | 22,571 | ⁴⁶ |
| precursor | | |
| LGALS1 Galectin-1 | 14,698 | ^{47, 48} |
| MMP2 72 kDa type IV collagenase precursor | 73,867 | ⁴⁹⁻⁵¹ |
| PEBP1 Phosphatidylethanolamine-binding | 21,039 | ⁵² |
| protein 1 | | |
| PLAUR Isoform 1 of Urokinase plasminogen | 36,959 | ^{38, 53, 54} |
| activator surface receptor precursor | | |
| RBP4 Plasma retinol-binding protein precursor | 22,992 | ⁵⁵ |
| SERPINA3 Isoform 1 of Alpha-1- | 50,583 | ³⁴ |
| antichymotrypsin precursor | | |
| SERPINE1 Plasminogen activator inhibitor 1 | 45,042 | ⁵⁶ |
| precursor | | |
| SERPINF2 Alpha-2-antiplasmin precursor | 55,047 | ⁵⁷ |
| SPP1 Isoform A of Osteopontin precursor | 35,405 | ⁵⁸⁻⁶⁰ |
| TF Serotransferrin precursor | 77,032 | ^{61, 62} |
| THBS1 Thrombospondin-1 precursor | 129,364 | ^{63, 64} |
| TIMP1 Metalloproteinase inhibitor 1 precursor | 23,153 | ⁶⁵ |
| TIMP2 Metalloproteinase inhibitor 2 precursor | 24,382 | ⁵¹ |
| TMEM110; ITIH4 Isoform 1 of Inter-alpha- | 103,308 | ⁶⁵ |
| trypsin inhibitor heavy chain H4 precursor | | |
| TTR Transthyretin precursor | 15,869 | ^{35, 55, 56, 62} |
| WFDC2 Isoform 1 of WAP four-disulfide core | 12,974 | ^{59, 66, 67} |
| domain protein 2 precursor | | |

¹For protein IPI accession numbers, see Table 3

TABLE 2
Panel of 52 Ovarian Cancer Biomarkers

| | Molecular |
|---|---|
| Protein Name | Mass, Da |
| AGRN Agrin precursor | 214,820 |
| BCAM Lutheran blood group glycoprotein precursor | 61,042 |
| C14orf141; LTBP2 Latent-transforming growth factor | 195,039 |
| beta-binding protein 2 precursor | |
| CD248 Isoform 1 of Endosialin precursor | 80,840 |
| CD59 CD59 glycoprotein precursor | 14,159 |
| CLU Clusterin precursor | 52,477 |
| COMP 80 kDa protein | 79,676 |
| CPA4 Carboxypeptidase A4 precursor | 47,334 |
| CST3 Cystatin-C precursor | 15,781 |
| CST6 Cystatin-M precursor | 16,493 |
| CTGF Isoform 1 of Connective tissue growth factor | 38,073 |
| precursor | |
| DAG1 Dystroglycan precursor | 97,563 |
| DKK3 Dickkopf-related protein 3 precursor | 38,272 |
| DSC2 Isoform 2A of Desmocollin-2 precursor | 99,945 |
| DSG2 desmoglein 2 preproprotein | 122,276 |
| ECM1 Extracellular matrix protein 1 precursor | 60,655 |
| EFEMP1 Isoform 1 of EGF-containing fibulin-like | 54,621 |
| extracellular matrix protein 1 precursor | |
| FAM3C Protein FAM3C precursor | 24,663 |
| FBLN1 Isoform C of Fibulin-1 precursor | 74,442 |
| FOLR1 Folate receptor alpha precursor | 29,801 |
| FSTL1 Follistatin-related protein 1 precursor | 34,967 |
| GLOD4 Uncharacterized protein C17orf25 | 54,995 |
| GM2A Ganglioside GM2 activator precursor | 20,805 |
| GPX3 Glutathione peroxidase 3 precursor | 25,488 |
| HSPG2 Basement membrane-specific heparan sulfate | 468,788 |
| proteoglycan core protein precursor | |
| HTRA1 Serine protease HTRA1 precursor | 51,269 |
| IGFBP2 Insulin-like growth factor-binding protein 2 | 35,119 |
| precursor | |
| IGFBP3 Insulin-like growth factor-binding protein 3 | 31,656 |
| precursor | |
| IGFBP4 Insulin-like growth factor-binding protein 4 | 27,916 |
| precursor | |
| IGFBP5 Insulin-like growth factor-binding protein 5 | 30,552 |
| precursor | |
| IGFBP6 Insulin-like growth factor-binding protein 6 | 25,304 |
| precursor | |
| IGFBP7 Insulin-like growth factor-binding protein 7 | 29,112 |
| precursor | |
| LRG1 Leucine-rich alpha-2-glycoprotein precursor | 38,162 |
| MST1 Hepatocyte growth factor-like protein precursor | 80,360 |
| MXRA5 Matrix-remodelling-associated protein 5 | 312,263 |
| precursor (Adlican) | |
| NID2 Nidogen-2 precursor | 151,377 |
| NPC2 Epididymal secretory protein E1 precursor | 16,552 |
| NUCB1 Nucleobindin-1 precursor | 53,862 |
| PCOLCE Procollagen C-endopeptidase enhancer 1 | 47,955 |
| precursor | |
| PLEC1 Isoform 1 of Plectin-1 | 531,708 |
| PLTP Isoform 1 of Phospholipid transfer protein | 54,723 |
| precursor | |
| PROCR Endothelial protein C receptor precursor | 30,697 |
| PROS1 Vitamin K-dependent protein S precursor | 75,105 |
| PSAP Isoform Sap-mu-0 of Proactivator polypeptide | 58,094 |
| precursor | |
| QSCN6 Isoform 1 of Sulfhydryl oxidase 1 precursor | 82,561 |
| SECTM1 Secreted and transmembrane protein 1 | 27,021 |
| precursor | |
| SERPINA6 Corticosteroid-binding globulin precursor | 45,124 |
| SOD1 16 kDa protein (Superoxide Dismutase 1) | 16,104 |
| SVEP1 polydom (Sel-Ob) | 390,478 |
| TAGLN2; CCDC19 Transgelin-2 | 22,374 |
| TGFBI Transforming growth factor-beta-induced protein | 74,665 |
| ig-h3 precursor | |
| VASN Vasorin precursor | 71,696 |

For protein IPI accession numbers, see Table 5.

### Discussion

One of the main obstacles in proteomic analysis of biological fluids is the presence of high-abundance proteins²⁶, mainly albumin and immunoglobulins. In human serum the top 10 most abundant proteins comprise over 95% of all proteins present in this fluid²⁷, and the top 20 are greater than 30 KDa in molecular mass. These abundant proteins, especially human serum albumin, generate massive amounts of ions which often result in the inaccurate representation and identification of ions from the low-abundance proteins, due to the limited number of ions passed on for tandem mass spectrometry analysis. Hence, these high abundance proteins must be depleted or removed in order to efficiently identify the proteins of low molecular mass and low abundance by mass spectrometry. Various methods have been previously used for the removal of albumin or immunoglobulins such as dye affinity resins or protein A/G beads^{28, 29}, yet these approaches are limited as albumin and other high-abundance proteins often act as transport proteins by binding (and thereby concentrating) many low-abundance proteins and peptides. Thus, removal of serum albumin and other abundant proteins may inadvertently remove many small proteins and peptides of interest³⁰.

Alternative approaches for biomarker discovery without the problems associated with high abundance molecules include analysis of tissue culture supernatants of cancer cell lines grown in serum-free media³¹. Also, recently, Faça et al. characterized the cell surface proteome and the proteins released into the extracellular milieu of three ovarian cancer cell lines, and identified over 6000 proteins as candidate biomarkers and therapeutic targets³².

By utilizing different separation methods, in combination with mass spectrometry, 445 proteins were identified within the soluble fraction of ascites fluid. Recently, the proteome of ascites fluid was reported by Gortzak-Uzan et al.¹⁷. Although they identified over 2200 proteins, only 229 were found in the soluble fraction of ascites. Since the serum proteome contains thousands of proteins, the list of 229 proteins id unlikely to represent the full proteome of soluble ascites fluid. As serum protein concentrations range over nine orders of magnitude, we aimed to identify a more extensive proteome of ascites fluid, focusing on low molecular mass (≦30 KDa).

Using different mobile phase systems (ammonium bicarbonate; phosphate/sulfate) and size exclusion chromatography, we identified 404 and 231 proteins, respectively. Combining these proteins with the 88 and 121 proteins identified from the 50 KDa and 100 KDa centrifugal ultrafiltration experiments, we identified a total of 445 unique proteins within ascites fluid, which is more than any other published proteome of soluble ascites and almost doubles the proteins reported earlier¹⁷. The results indicate that by combining different sample fractionation methods, a greater coverage of the ascites fluid proteome can be obtained, thus allowing for better biomarker selection. The overlap between the soluble ascites fluid proteome described herein (445 proteins) and that of Gortzak-Uzan¹⁷ (220 proteins) was 28% (Table 3).

Although ascites fluid is the build-up of peritoneal fluid accumulated from infiltrated circulating serum, its composition may be different due to the presence of the burdening ovarian tumor. By comparing the identified ovarian ascites fluid subproteome with the human plasma proteome database, only 34% of the proteins were common. Even taking into account that the plasma proteome is not complete, and the focus was on low molecular mass proteins, the data suggest that ascites fluid has a significantly different composition than plasma and its proteins reflect the contribution of the tumor microenvironment.

Classification of the identified proteins by Swiss Prot, Genome Ontology, as well as other publicly available databases, indicated that 52% of the proteins within ascites fluid are extracellular or membranous (FIG. 4), as would be expected for an extracellular biological fluid. With over half of the proteins defined as extracellular or membranous, it is highly plausible that many of these proteins are shed by the tumor cells, allowing for an efficient selection of candidate ovarian cancer biomarkers.

The proteins identified within ascites fluid reflect the pathobiological state of ovarian cancer. Since ascites accumulation is often linked to advanced ovarian cancer, it is likely that many of these identified proteins represent promising new biomarkers. On the other hand, not all proteins in ascites represent tumor-associated antigens. By applying an arbitrary set of selection criteria, we were able to minimize the list of candidate biomarker proteins to a more manageable number (approximately 50) for further selection and validation. From the list of extracellular and membranous proteins, we eliminated high abundance proteins, proteins previously studied as serological biomarkers for ovarian cancer, proteins identified with a single unique peptide from only one fractionation protocol and proteins that were not identified in at least one supernatant of four different ovarian cancer cell lines. The identification of 25 known secreted or membrane bound ovarian cancer biomarkers (Table 1) supports the view that the outlined approach can identify novel biomarkers.

From the panel of 52 candidate biomarkers (Table 2), 31 proteins were also identified within the ascites fluid proteome by Gortzak-Uzan et al.¹⁷ (Table 5). However, these authors did not select any of these proteins for further investigation as they had applied a different set of criteria for biomarker selection. This underlines the fact that despite successful identification of proteins in fluids by mass spectrometry, the criteria for narrowing down the list of candidates are also of paramount importance.

While many of the filtering criteria were somewhat subjective, the discovery strategy appears efficient as 25 of the 289 identified extracellular or membranous proteins were previously studied as serum ovarian cancer biomarkers (Table 1). While the most widely studied biomarker for ovarian cancer, CA125, was not identified in any of the experiments, this can be explained by the fact that CA125 is highly glycosylated with a molecular mass ranging from 190 to 2700 KDa³³, and therefore excluded during sample preparation. Other glycosylated proteins of molecular mass of ≧30 KDa may have not been identified. Additionally, many of the candidates (Table 2) have molecular masses ≧30 KDa, implying that they are likely fragmented in ascites fluid. This has also been reported by Zhang et al. who observed truncated forms of transthyretin and cleavage fragment of inter-³⁴-trypsin inhibitor heavy chain H4

### Example 2

The major challenge in biomarker discovery using proteomics is the validation phase. Candidate ovarian cancer biomarkers are validated using ELISA assays or other quantitative techniques, and serum as the fluid of choice.

Biomarkers are validated either using commercially available or in house developed ‘sandwich type’ ELISA or the PIM (product ion monitoring) assay. The PIM method is performed for example as described in Kulasingam V, Smith C R, Batruch I, Buckler A, Jeffery D A, Diamandis E P. “Product ion monitoring” assay for prostate-specific antigen in serum using a linear ion-trap. J Proteome Res 2008 7:640-647.

Available antibodies are commercially purchased and the working concentration is identified. The level of the biomarker is measured using the above-mentioned experimental techniques in serum samples from ovarian cancer patients, normal patients and patients with benign gynecological diseases diluted to the appropriate concentrations (i.e. 1:200 or 1:500). A number of serum samples are analyzed, for example, 100 normal serum, 100 cancer serum and 100 benign gynecological diseases serum samples. The levels of the biomarker in each data set are then calculated and statistical analysis is performed to identify whether a significant difference is present.

### Example 3

### Nidogen-2 Biomarker Verification and Nidogen-2 ELISA Assay

The concentration of nidogen-2 in serum was measured in 100 serum samples from normal (e.g. women without ovarian cancer) women, 100 serum samples from women with ovarian cancer of various stages and 100 serum samples from women with benign gynecological diseases.

The concentration of nidogen-2 was assayed using a highly sensitive and specific non-competitive ‘sandwich-type’ ELISA developed in-house with commercially available antibodies from R&D systems (Minneapolis, Minn.). Goat polyclonal anti-human nidogen-2 antibody was immobilized in a 96-well white polystyrene plate by incubating 200 ng/100 μl/well in a coating buffer (50 mmol/L Tris, 0.05% sodium azide; pH 7.8) overnight. After washing three times with washing buffer (5 mmol/L Tris, 150 mmol/L NaCl, 0.05% Tween 20; pH 7.8), 50 μl of each serum sample diluted 1:200 in 6% bovine serum albumin (BSA) or 50 μl of nidogen-2 standards were pipetted into each well, in addition to 50 μl of assay buffer (50 mmol/L Tris, 6% BSA, 0.01% goat IgG, 0.005% mouse IgG, 0.1% bovine IgG, 0.5 mol/L KCl, 0.05% sodium azide; pH 7.8) and incubated for 1.5 hour with shaking at room temperature. The plates were washed six times with the washing buffer, after which biotinylated detection antibody solution (100 μl; 25 ng goat polyclonal anti-human nidogen-2 antibody in assay buffer) was added to each well and incubated for 1 hour at room temperature with shaking. The plates were then washed six times with the washing buffer. Subsequently, alkaline phosphatase-conjugated streptavidin solution (5 ng/well; Jackson ImmunoResearch, Westgrove, Pa.) in 6% BSA buffer (in 50 mmol/L Tris, 0.05% sodium azide; pH 7.8) were added to each well and incubated for 15 min with shaking, at room temperature. The plates were washed six times with the washing buffer and substrate buffer (100 μl; 0.1 mol/L Iris buffer; pH 9.1) containing 1 mmol/L of the substrate diflunisal phosphate, 0.1 mol/L NaCl, and 1 mmol/L MgCl₂ was added to each well and incubated for 10 min with shaking at room temperature. After adding 50 μl of developing solution containing Tb³⁺/EDTA complex, the fluorescence of each well was measured with the Wallac Envision 2103 multilabel reader.

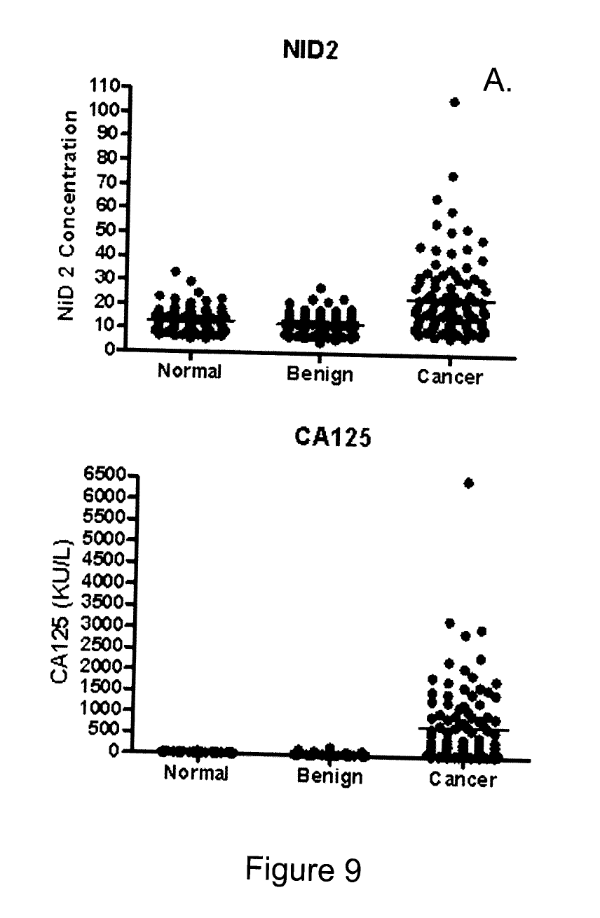

The concentration of nidogen-2 was graphed using Graphpad Prism. CA125 levels were also analyzed and compared with nidogen-2. The results are shown in FIGS. 9-13. The concentration of nidogen-2 is in micrograms/L.

Clinical Samples: 100 serum samples from ovarian cancer patients (ages 33 to 82 years; median, 57.5 years), 100 serum samples from normal, apparently healthy women (ages 25 to 88 years; median, 51.5 years), and 100 serum samples from women with benign gynecological diseases (ages 20 to 80 years; median, 38 years). Serum samples were stored in −80° C. until further analysis. Of the 100 ovarian carcinoma patients, 38 were stage 1, 19 were stage 2, 31 were stage 3, and 12 were stage 4 and 1 case was unknown. 59 samples were type serous cystadenocarcinoma of the ovary, 19 type mucinous cystadenocarcinoma of the ovary, 11 type endometrioid adenocarcinoma of the ovary and 10 type clear cell carcinoma of the ovary. The CA125 levels were measured using the commercially available ELISA assay by Roche.

### Results:

The concentration of nidogen-2 and CA125 are shown in FIGS. 9A and 9B. Both CA125 and nidogen-2 are elevated in ovarian cancer serum samples and not in normal and benign conditions.

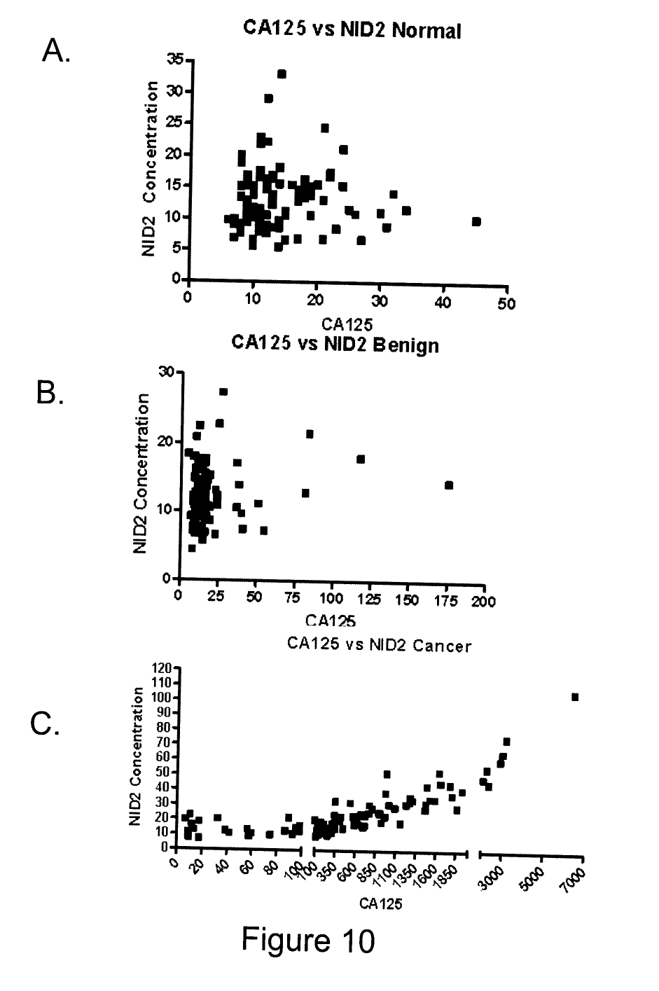

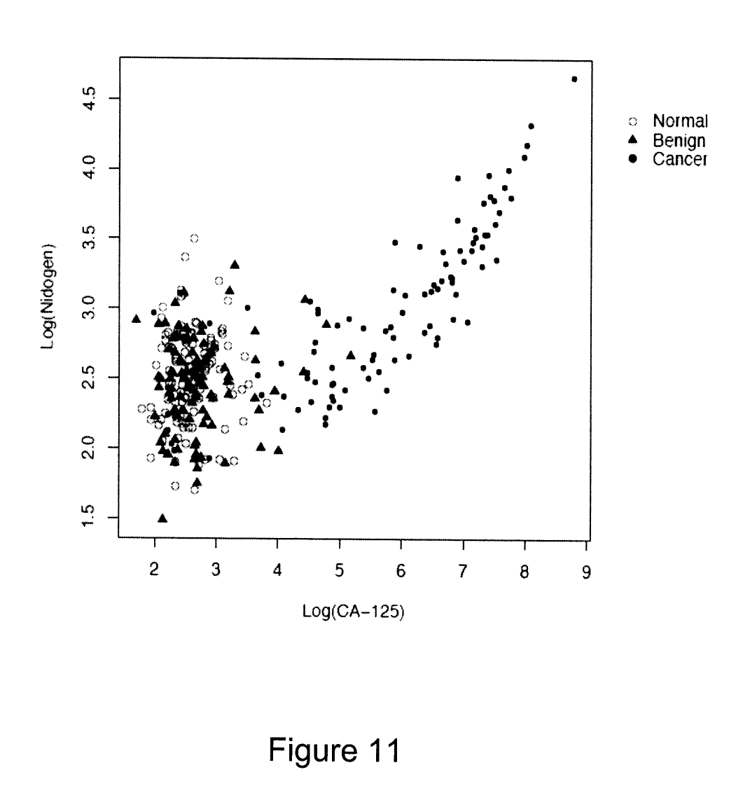

The correlation between nidogen-2 and CA125 is shown in normal (FIG. 10A), benign (FIG. 10B) and ovarian cancer (FIG. 10 C) sample sets. Nidogen-2 is shown to correlate with CA125 in cancer (FIG. 11). The Spearman's correlation coefficient between them is 0.46 (p<0.001) across all samples.

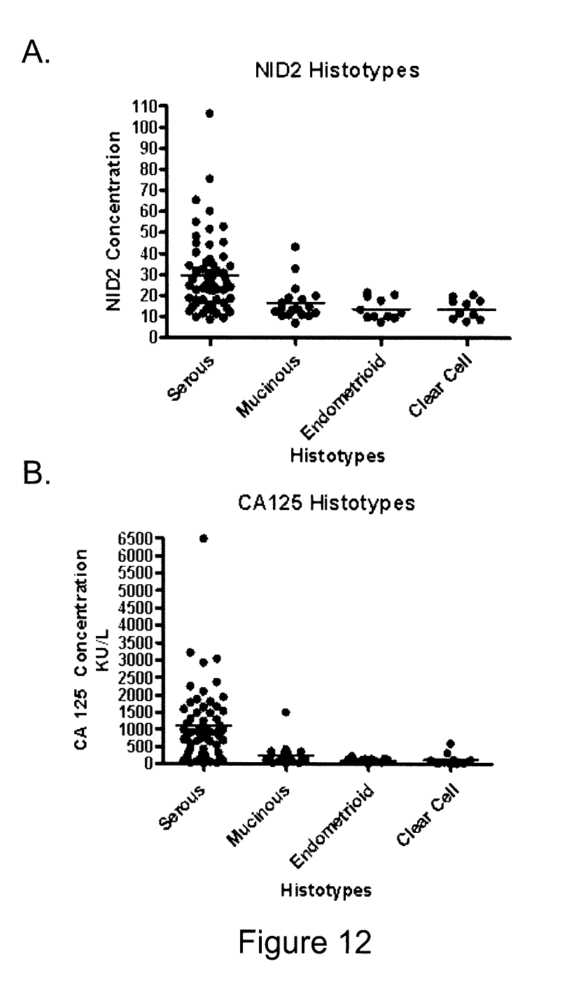

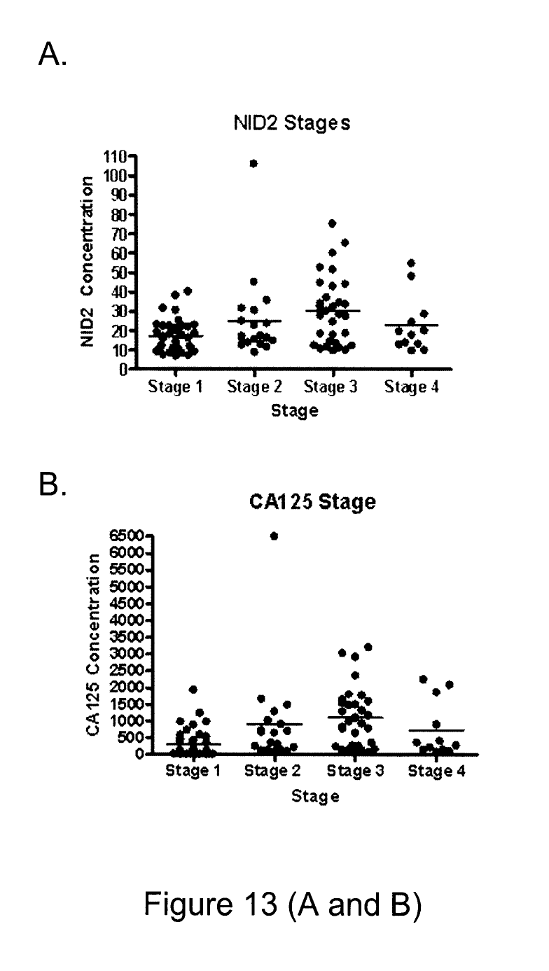

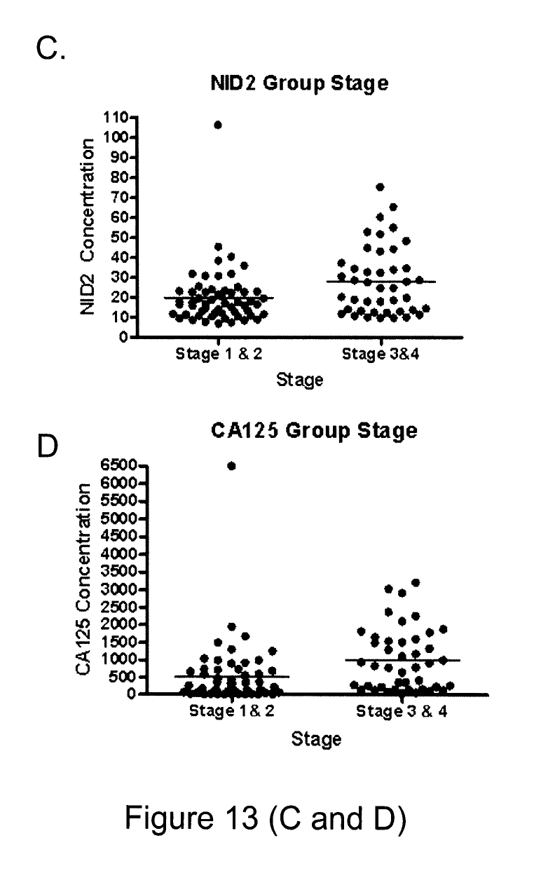

Both nidogen-2 and CA125 are elevated in patients with serous cystadenocarcinoma (FIG. 12A, FIG. 12B). Similarly, nidogen-2 and CA125 are both elevated in late stage (stage 3 and 4) ovarian cancer as opposed to early stage (stage 1 and 2), shown in FIGS. 13A-D.

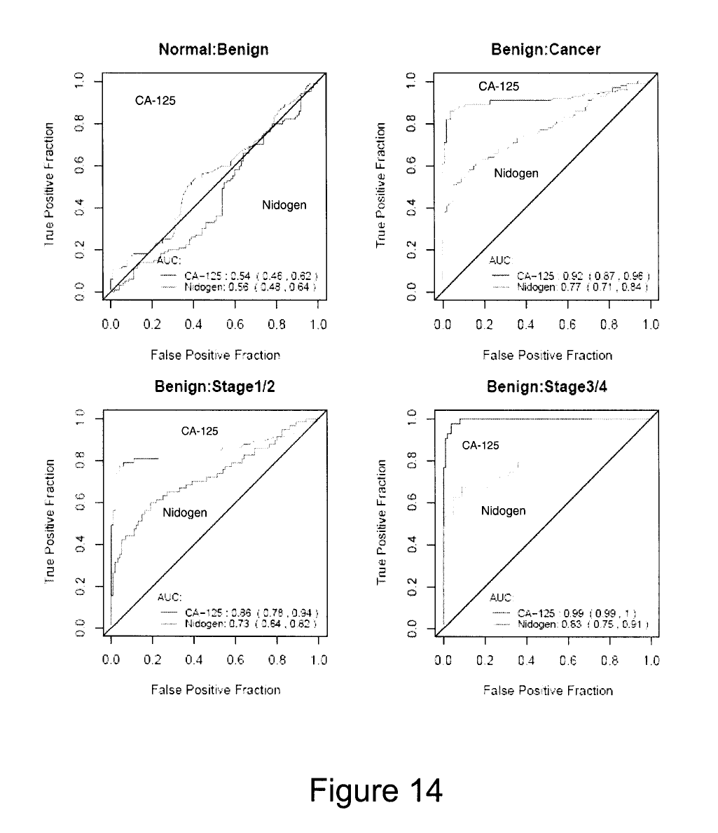

Receiver Operating Characteristic (ROC) curves for single marker of nidogen-2 or CA125 with estimated AUC (95% Cl). Normal patients versus cancer patients (FIG. 14).

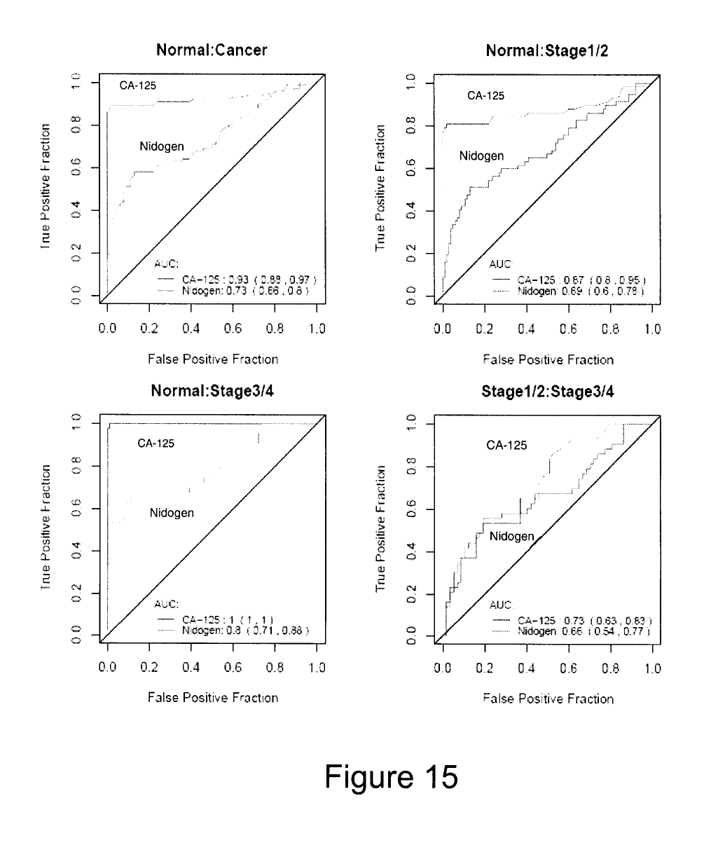

ROC curves for single marker of nidogen-2 or CA125 with estimated area under the curve (AUC; 95% confidence interval; Cl). Benign disease patients versus ovarian cancer patients (FIG. 15). The ROC curves display the true positive fraction of patients versus the false positive fraction of patients at each cutoff point. Nidogen-2 also bears significant diagnostic value because the AUC is greater than 0.50. A marker with an AUC of 0.50 is not informative.

TABLE 3
Extracellular and Membranous proteins identified within the subproteome of ascites fluid.
This table includes data from all four fraction methods.

| | Protein | Protein Molecular | | # Unique Peptides | | | | | | Total Unique |
|---|---|---|---|---|---|---|---|---|---|---|
| Protein Name | Accession Numbers | Weight (AMU) | Plasma Proteome | 50K | 100K | NH4HCO3 | NH4HCO3#2 | PO4SO4 | PO4SO4#2 | Peptides |
| A1BG Alpha-1B-glycoprotein precursor | IPI00022895 | 54,254.40 | Y | 8 | 6 | 32 | 31 | 37 | 24 | 138 |
| A2M Alpha-2-macroglobulin precursor | IPI00478003 | 163,258.80 | | 15 | 8 | 69 | 26 | 85 | 53 | 256 |
| ADAMDEC1 ADAM DEC1 precursor | IPI00004480 | 52,758.00 | | 0 | 0 | 2 | 0 | 0 | 0 | 2 |
| ADAMTS1 ADAMTS-1 precursor | IPI00005908 | 105,339.70 | | 0 | 0 | 1 | 2 | 0 | 0 | 3 |
| AFM Afamin precursor | IPI00019943 | 69,052.10 | Y | 3 | 1 | 15 | 5 | 11 | 12 | 49 |
| AGRN Agrin precursor | IPI00374563, | 214,820.00 | | 0 | 0 | 8 | 7 | 3 | 8 | 18 |
| | IPI00795766 | | | | | | | | | |
| AGT Angiotensinogen precursor | IPI00032220 | 53,136.80 | Y | 5 | 6 | 17 | 11 | 17 | 15 | 71 |
| AHSG Alpha-2-HS-glycoprotein precursor | IPI00022431 | 39,305.40 | Y | 4 | 0 | 20 | 17 | 26 | 17 | 84 |
| AKR1A1 Alcohol dehydrogenase | IPI00220271 | 36,555.60 | | 0 | 0 | 5 | 0 | 0 | 0 | 5 |
| AMBP AMBP protein precursor AMY1A; AMY2A; AMY1B; AMY1C | IPI00022426 | 38,981.50 | Y | 5 | 8 | 31 | 14 | 25 | 14 | 84 |
| Salivary alpha-amylase precursor | | | | | | | | | | |
| AMY2A Pancreatic alpha-amylase precursor | IPI00025476 | 57,689.40 | | 0 | 0 | 2 | 8 | 0 | 0 | 2 |
| APCS Serum amyloid P-component precursor | IPI00022391 | 25,369.70 | Y | 0 | 0 | 8 | 0 | 0 | 0 | 8 |
| APOA1 Apolipoprotein A-I precursor | IPI00021841 | 30,760.50 | Y | 21 | 8 | 104 | 67 | 62 | 57 | 319 |
| APOA2 Apolipoprotein A-II precursor | IPI00021854 | 11,157.20 | Y | 5 | 0 | 22 | 16 | 12 | 12 | 67 |
| APOA4 Apolipoprotein A-IV precursor | IPI00304273, | 45,381.30 | Y | 26 | 0 | 67 | 54 | 5 | 29 | 200 |
| | IPI00784338 | | | | | | | | | |
| APOB Apolipoprotein B-100 precursor | IPI00022229 | 515,554.20 | Y | 2 | 6 | 36 | 37 | 9 | 15 | 105 |
| APOC1 Apolipoprotein C-I precursor | IPI00021855 | 9,314.30 | Y | 1 | 0 | 4 | 10 | 0 | 8 | 15 |
| APOC2; APOC4 Apolipoprotein C-II precursor | IPI00021856 | 11,266.10 | Y | 2 | 0 | 8 | 15 | 0 | 0 | 27 |
| APOC3 Apolipoprotein C-III precursor | IPI00021857, | 10,834.30 | Y | 3 | 2 | 11 | 15 | 0 | 0 | 29 |
| | IPI00657670 | | | | | | | | | |
| APOD Apolipoprotein D precursor | IPI00006662 | 21,258.00 | Y | 0 | 0 | 25 | 19 | 6 | 4 | 56 |
| APOE Apolipoprotein E precursor | IPI00021842 | 36,135.50 | Y | 9 | 0 | 39 | 45 | 10 | 5 | 114 |
| APOF apolipoprotein F precursor | IPI00299435 | 35,382.30 | Y | 2 | 0 | 6 | 4 | 0 | 0 | 16 |
| APOH Beta-2-glycoprotein 1 precursor | IPI00298828 | 38,280.50 | Y | 7 | 4 | 51 | 43 | 49 | 36 | 190 |
| APOL1 Isoform 2 of Apolipoprotein-L1 precursor | IPI00186903, | 45,854.70 | Y | 0 | 0 | 6 | 6 | 0 | 0 | 12 |
| | IPI00514475 | | | | | | | | | |
| APOM Apolipoprotein M | IPI00030739 | 21,235.90 | Y | 0 | 0 | 6 | 0 | 3 | 0 | 9 |
| ATP6AP2 Protein | IPI00168884, | 26,615.30 | | 0 | 0 | 2 | 0 | 0 | 0 | 2 |
| | IPI00642797, | | | | | | | | | |
| | IPI00828107 | | | | | | | | | |
| AZGP1 alpha-2-glycoprotein 1, zinc | IPI00166729 | 34,240.60 | Y | 4 | 0 | 8 | 3 | 22 | 20 | 62 |
| B2M Beta-2-microglobulin precursor | IPI00004656, | 13,696.90 | | 1 | 0 | 10 | 16 | 7 | 0 | 40 |
| | IPI00796379 | | | | | | | | | |
| BCAM Lutheran blood group glycoprotem precursor | IPI00794214 | 61,041.90 | | 0 | 0 | 4 | 0 | 0 | 0 | 4 |
| C14orf141; LTBP2 Latent-transforming growth factor beta-bindin [symbol] | IPI00292150 | 195,038.50 | | 0 | 0 | 2 | 4 | 0 | 0 | 6 |
| C1QB complement component 1, q subcomponent, B chain prec [symbol] | IPI00477992 | 26,704.80 | | 0 | 0 | 0 | 3 | 6 | 3 | 12 |
| C1QC Complement C1q subcomponent subunit C precursor | IPI00022394 | 25,756.00 | Y | 0 | 0 | 0 | 0 | 4 | 4 | 8 |
| C1R Complement C1r subcomponent precursor | IPI00296165 | 80,156.70 | Y | 0 | 0 | 32 | 12 | 28 | 11 | 83 |
| C1S Complement C1s subcomponent precursor | IPI00017696 | 76,666.20 | Y | 0 | 0 | 15 | 6 | 2 | 3 | 26 |
| C2 Complement C2 precursor (Fragment) | IPI00303963 | 83,250.80 | Y | 3 | 4 | 19 | 14 | 16 | 9 | 64 |
| C3 Complement C3 precursor (Fragment) | IPI00783987 | 187,131.10 | | 43 | 31 | 258 | 123 | 251 | 197 | 903 |
| C4A Complement component 4A | IPI00543525 | 192,725.70 | | 0 | 2 | 0 | 0 | 0 | 4 | 6 |
| C4B complement component 4B preproprotein | IPI00418163 | 192,734.80 | | 23 | 15 | 156 | 71 | 147 | 132 | 544 |
| C48PA C4b-binding protein alpha chain precursor | IPI00021727 | 67,015.00 | Y | 0 | 0 | 24 | 0 | 8 | 1 | 35 |
| C4BPB Isoform 1 of C4b-binding protein beta chain precursor | IPI00025862, | 28,339.20 | Y | 0 | 0 | 3 | 0 | 0 | 0 | 3 |
| | IPI00555752 | | | | | | | | | |
| C5 Complement C5 precursor | IPI00032291 | 188,317.40 | Y | 0 | 2 | 42 | 27 | 53 | 42 | 166 |
| C6 Complement component C6 precursor | IPI00009920 | 105,734.20 | Y | 0 | 4 | 29 | 24 | 5 | 17 | 89 |
| C8B Complement component C8 beta chain precursor | IPI00294395 | 67,028.90 | Y | 0 | 0 | 0 | 52 | 25 | 28 | 105 |
| C8G Complement component C8 gamma chain precursor | IPI00011261 | 22,259.30 | Y | 0 | 0 | 8 | 19 | 13 | 13 | 53 |
| C9 Complement component C9 precursor | IPI00022395 | 63,156.80 | Y | 3 | 6 | 52 | 34 | 22 | 27 | 139 |
| CACNA1S Voltage-dependent L-type calcium channel subunit alp [symbol] | IPI00299240, | 212,548.40 | | 0 | 1 | 0 | 0 | 0 | 0 | 1 |
| | IPI00514837, | | | | | | | | | |
| | IPI00642897 | | | | | | | | | |
| CADM3; DARC Isoform 2 of Immunoglobulin superfamily membe [symbol] | IPI00009619, | 47,003.80 | | 0 | 0 | 2 | 0 | 0 | 8 | 2 |
| | IPI00166048 | | | | | | | | | |
| CD14 Monocyte differentiation antigen CD14 precursor | IPI00029260 | 40,058.60 | Y | 6 | 2 | 19 | 20 | 21 | 16 | 84 |
| CD248 Isoform 1 of Endosialin precursor | IPI00006971 | 80,839.70 | | 0 | 0 | 2 | 0 | 0 | 0 | 2 |
| CD59 CD59 glycoprotein precursor | IPI00011302 | 14,159.70 | | 0 | 0 | 2 | 0 | 0 | 0 | 2 |
| CD5L CD5 antigen-like precursor | IPI00025204 | 38,067.90 | Y | 0 | 0 | 0 | 0 | 2 | 0 | 2 |
| CDH2 Cadherin-2 precursor | IPI00290085 | 99,793.80 | Y | 0 | 0 | 2 | 0 | 0 | 0 | 2 |
| CETP Isoform 1 of Cholesteryl ester transfer protein precursor | IPI00006173, | 54,739.20 | Y | 0 | 0 | 5 | 0 | 3 | 0 | 8 |
| | IPI00641481 | | | | | | | | | |
| CFB Isoform 1 of Complement factor B precursor (Fragment) | IPI00019591 | 85,515.20 | Y | 10 | 10 | 112 | 65 | 60 | 44 | 301 |
| CFD Complement factor D precursor | IPI00019579, | 26,985.30 | Y | 3 | 0 | 2 | 21 | 8 | 2 | 36 |
| | IPI00165972 | | | | | | | | | |
| CFH Isoform 1 of Complement factor H precursor | IPI00029739 | 139,052.10 | Y | 5 | 5 | 91 | 31 | 54 | 71 | 257 |
| CFHR1 Complement factor H-related protein1 precursor | IPI00011264 | 37,643.30 | Y | 0 | 0 | 0 | 20 | 11 | 10 | 39 |
| CFHR2 Isoform Long of Complement factor H-related protein 2 | IPI00006154, | 30,632.60 | Y | 0 | 0 | 0 | 2 | 0 | 2 | 4 |
| | IPI00218949 | | | | | | | | | |
| CFHR3 Complement factor H-related protein 3 precursor | IPI00027507 | 37,305.40 | Y | 0 | 0 | 2 | 0 | 5 | 0 | 7 |
| CFI Complement factor I precursor | IPI00291867 | 65,701.80 | Y | 0 | 0 | 59 | 9 | 37 | 25 | 138 |
| CHI3L1 Chitinase-3-like protein 1 precursor | IPI00002147 | 42,609.00 | | 0 | 0 | 0 | 10 | 9 | 4 | 23 |
| CILP Cartilage intermediate layer protein 1 precursor | IPI00289275, | 132,533.20 | | 0 | 0 | 5 | 0 | 1 | 2 | 8 |
| | IPI00791803 | | | | | | | | | |
| CLEC3B Hypothetical protein DKFZp686H17246 | IPI00792115 | 17,776.20 | | 0 | 3 | 15 | 10 | 15 | 7 | 50 |
| CLU Clusterin precursor | IPI00291262, | 52,476.90 | Y | 0 | 4 | 12 | 14 | 4 | 11 | 45 |
| | IPI00400826, | | | | | | | | | |
| | IPI00795633 | | | | | | | | | |
| CNDP1 Beta-Ala-His dipeptidase precursor | IPI00064667 | 56,675.10 | Y | 2 | 0 | 8 | 4 | 0 | 0 | 14 |
| CNNM3 cyclin M3 isoform 1 | IPI00168565, | 76,102.50 | | 0 | 3 | 0 | 0 | 0 | 0 | 2 |
| | IPI00386115 | | | | | | | | | |
| COL1A1 Collagen alpha-1(I) chain precursor | IPI00297646 | 138,893.40 | | 3 | 2 | 18 | 4 | 6 | 2 | 35 |
| COL1A2 Collagen alpha-2(I) chain precursor | IPI00304962 | 129,270.60 | | 0 | 0 | 6 | 0 | 0 | 0 | 6 |
| COL4A2 Collagen alpha-2(IV) chain precursor | IPI00306322, | 167,521.70 | | 0 | 0 | 4 | 0 | 0 | 0 | 4 |
| | IPI00477950 | | | | | | | | | |
| COL5A1 Collagen alpha-1(V) chain precursor | IPI00477611 | 183,593.60 | | 4 | 0 | 18 | 12 | 7 | 1 | 42 |
| COL6A1 Collagen alpha-1(VI) chain precursor | IPI00291136 | 108,512.90 | | 0 | 2 | 29 | 0 | 22 | 8 | 61 |
| COL6A3 alpha 3 type VI collagen isoform 1 precursor | IPI00022200, | 343,649.40 | Y | 3 | 3 | 36 | 29 | 16 | 7 | 94 |
| | IPI00376964 | | | | | | | | | |
| Cold agglutinin FS-1 L-chain (Fragment) | IPI00827773 | 12,370.50 | | 0 | 0 | 0 | 0 | 2 | 0 | 2 |
| COMP 80 kDa protein | IPI00643348 | 79,676.20 | | 0 | 0 | 0 | 0 | 0 | 2 | 2 |
| CP 97 kDa protein | IPI00794184 | 97,051.70 | | 0 | 0 | 0 | 0 | 0 | 4 | 4 |
| CP Ceruloplasmin precursor | IPI00017601 | 122,189.90 | Y | 12 | 0 | 133 | 16 | 99 | 81 | 348 |
| CPA4 Carboxypeptidase A4 precursor | IPI00008894 | 47,334.40 | | 0 | 0 | 4 | 0 | 0 | 0 | 4 |
| CPB2 Isoform 1 of Carboxypeptidase B2 precursor | IPI00329775 | 48,394.60 | Y | 0 | 0 | 9 | 4 | 3 | 0 | 16 |
| CPN1 Carboxypeptidase N catalytic chain precursor | IPI00010295 | 52,268.30 | Y | 0 | 0 | 5 | 9 | 0 | 0 | 14 |
| CPN2 similar to Carboxypeptidase N subunit 2 precursor | IPI00738433 | 60,568.80 | | 0 | 0 | 4 | 9 | 2 | 1 | 18 |
| CRP Isoform 1 of C-reactive protein precursor | IPI00022389 | 25,021.00 | Y | 0 | 0 | 5 | 3 | 4 | 6 | 18 |
| CRTAC1 Isoform 1 of Cartilage acidic protein 1 precursor | IPI00451624, | 71,403.00 | | 0 | 2 | 6 | 0 | 0 | 0 | 6 |
| | IPI00451625 | | | | | | | | | |
| CST3 Cystatin-C precursor | IPI00032293 | 15,781.20 | Y | 3 | 0 | 0 | 14 | 0 | 0 | 19 |
| CST6 Cystatin-M precursor | IPI00019954, | 16,493.10 | | 0 | 0 | 2 | 0 | 0 | 0 | 2 |
| | IPI00788184 | | | | | | | | | |
| CTGF Isoform 1 of Connective tissue growth factor precursor | IPI00020977, | 38,072.70 | | 0 | 0 | 0 | 7 | 0 | 0 | 2 |
| | IPI00220647 | | | | | | | | | |
| CTHRC1 Isoform 2 of Collagen triple helix repeat-containing pro [symbol] | IPI00060423, | 25,145.40 | | 0 | 0 | 2 | 0 | 0 | 0 | 2 |
| | IPI00336091 | | | | | | | | | |
| CTSB Cathepsin B precursor | IPI00295741 | 37,803.20 | | 0 | 0 | 17 | 0 | 6 | 5 | 28 |
| CTSD Cathepsin D precursor | IPI00011229, | 44,535.00 | Y | 0 | 0 | 3 | 0 | 0 | 0 | 3 |
| | IPI00658053 | | | | | | | | | |
| CTSL1 Cathepsin L precursor | IPI00012887 | 37,546.10 | | 0 | 0 | 2 | 0 | 0 | 0 | 2 |
| DAG1 Dystroglycan precursor | IPI00028911 | 97,563.30 | Y | 0 | 0 | 5 | 0 | 0 | 0 | 5 |
| DKK3 Dickkopf-related protein 3 precursor | IPI00002714, | 38,272.30 | | 0 | 0 | 2 | 0 | 0 | 0 | 2 |
| | IPI00383937 | | | | | | | | | |
| DSC2 Isoform 2A of Desmocollin-2 precursor | IPI00025846, | 99,944.60 | | 0 | 0 | 2 | 0 | 0 | 0 | 2 |
| | IPI00220146 | | | | | | | | | |
| DSG2 desmoglein 2 preproprotein | IPI00028931 | 122,276.40 | Y | 0 | 0 | 3 | 0 | 0 | 0 | 8 |
| ECM1 Extracellular matrix protein 1 precursor | IPI00003351, | 60,655.40 | Y | 0 | 2 | 16 | 0 | 25 | 17 | 60 |
| | IPI00645849 | | | | | | | | | |
| EFEMP1 Isoform 1 of EGF-containing fibulin-like extracellular m [symbol] | IPI00029658, | 54,621.10 | | 0 | 4 | 28 | 6 | 25 | 16 | 78 |
| | IPI00220813, | | | | | | | | | |
| | IPI00220814, | | | | | | | | | |
| | IPI00220815 | | | | | | | | | |
| EFNB2 Ephrin-B2 precursor | IPI00005126 | 36,906.10 | | 0 | 0 | 1 | 0 | 0 | 0 | 1 |
| ENO1 Isoform alpha-enolase of Alpha-enolase | IPI00465248 | 47,152.20 | | 2 | 4 | 6 | 14 | 0 | 0 | 26 |
| ENPP2 Isoform 1 of Ectonucleotide pyrophosphatase/phosphodi [symbol] | IPI00156171, | 98,987.40 | | 0 | 0 | 2 | 8 | 0 | 0 | 2 |
| | IPI00303210 | | | | | | | | | |
| ERC1 Isoform 1 of ELKS/RAB6-interacting/CAST family member | IPI00216719, | 128,072.60 | | 0 | 0 | 1 | 0 | 0 | 0 | 1 |
| | IPI00374976 | | | | | | | | | |
| F11 Isoform 1 of Coagulation factor XI precursor | IPI00008556, | 70,091.20 | Y | 0 | 0 | 0 | 0 | 0 | 9 | 9 |
| | IPI00216588 | | | | | | | | | |
| F12 Coagulation factor XII | IPI00783169 | 67,616.40 | | 0 | 0 | 9 | 15 | 8 | 2 | 34 |
| F13B Coagulation factor XIII B chain precursor | IPI00007240 | 75,474.60 | Y | 0 | 0 | 5 | 0 | 13 | 13 | 31 |
| F2 Prothrombin precursor (Fragment) | IPI00019568 | 70,018.80 | Y | 4 | 7 | 80 | 60 | 44 | 16 | 233 |
| FAM3C Protein FAM3C precursor | IPI00021923 | 24,662.90 | | 0 | 0 | 4 | 5 | 2 | 2 | 15 |
| FBLN1 Isoform C of Fibulin-1 precursor | IPI00296537 | 74,441.90 | Y | 0 | 0 | 3 | 0 | 5 | 2 | 10 |
| FBLN1 Isoform D of Fibulin-1 precursor | IPI00296534 | 77,240.80 | Y | 0 | 0 | 34 | 0 | 29 | 28 | 91 |
| FCN3 Isoform 1 of Ficolin-3 precursor | IPI00293925, | 32,885.30 | Y | 0 | 0 | 0 | 0 | 2 | 0 | 2 |
| | IPI00419744 | | | | | | | | | |
| FETUB Fetuin-B precursor | IPI00005439 | 42,037.40 | Y | 0 | 2 | 16 | 4 | 20 | 12 | 54 |
| FGA Isoform 1 of Fibrinogen alpha chain precursor | IPI00021885 | 94,955.40 | Y | 15 | 4 | 68 | 40 | 36 | 47 | 205 |
| FGB Fibrinogen beta chain precursor | IPI00298497 | 55,910.60 | Y | 13 | 6 | 62 | 23 | 89 | 53 | 256 |
| FGG Isoform Gamma-B of Fibrinogen gamma chain precursor | IPI00021891 | 51,495.30 | Y | 11 | 4 | 56 | 14 | 75 | 53 | 212 |
| FN1 Isoform 1 of Fibronectin precursor | IPI00022418 | 262,581.00 | Y | 14 | 13 | 134 | 75 | 94 | 98 | 429 |
| FOLR1 Folate receptor alpha precursor | IPI00441498 | 29,800.50 | | 0 | 0 | 4 | 0 | 0 | 0 | 4 |
| FOLR2 Folate receptor beta precursor | IPI00784257 | 31,141.30 | | 0 | 0 | 5 | 0 | 0 | 0 | 5 |
| FSTL1 Follistatin-related protein 1 precursor | IPI00029723 | 34,967.30 | | 0 | 8 | 4 | 0 | 0 | 0 | 4 |
| GAPDH Glyceraldehyde-3-phosphate dehydrogenase | IPI00219018 | 36,035.30 | Y | 5 | 3 | 14 | 10 | 0 | 0 | 32 |
| GC Vitamin D-binding protein precursor | IPI00555812 | 52,946.60 | | 12 | 0 | 64 | 18 | 66 | 51 | 219 |
| GDI2 GDP dissociation inhibitor 2 | IPI00645255 | 29,839.90 | | 0 | 0 | 0 | 2 | 3 | 0 | 5 |
| GKN1 Gastrokine-1 precursor | IPI00021342, | 20,313.10 | | 0 | 0 | 2 | 0 | 0 | 0 | 2 |
| | IPI00749381 | | | | | | | | | |
| GLOD4 Uncharacterized protein C17orf25 | IPI00007102, | 54,995.30 | | 0 | 0 | 2 | 0 | 0 | 0 | 2 |
| | IPI00032575, | | | | | | | | | |
| | IPI00745272 | | | | | | | | | |
| GM2A Ganglioside GM2 activator precursor | IPI00018236 | 20,804.90 | | 0 | 0 | 3 | 0 | 0 | 0 | 3 |
| GOLPH2 Golgi phosphoprotein 2 | IPI00171411, | 46,254.70 | | 0 | 0 | 1 | 0 | 0 | 0 | 1 |
| | IPI00759659, | | | | | | | | | |
| | IPI00784293 | | | | | | | | | |
| GPX3 Glutathione peroxidase 3 precursor | IPI00026199 | 25,488.20 | Y | 2 | 2 | 7 | 9 | 0 | 0 | 20 |
| GRN Isoform 2 of Granulins precursor | IPI00182138, | 47,156.20 | | 0 | 0 | 2 | 0 | 0 | 0 | 2 |
| | IPI00296713 | | | | | | | | | |
| GSN Isoform 1 of Gelsolin precursor | IPI00026314 | 85,679.80 | Y | 6 | 4 | 57 | 41 | 29 | 37 | 184 |
| HABP2 Hyaluronan-binding protein 2 precursor | IPI00041065 | 62,653.40 | Y | 0 | 0 | 0 | 0 | 8 | 2 | 10 |
| HGFAC Hepatocyte growth factor activator precursor | IPI00029193 | 70,663.50 | Y | 0 | 0 | 3 | 4 | 0 | 0 | 7 |
| HLA-A HLA class I histocompatibility antigen, A-23 alpha chain p [symbol] | IPI00472151 | 40,714.20 | | 0 | 0 | 6 | 4 | 0 | 0 | 10 |
| HLA-C; HLA-B; MICA; LOC730410 HLA class I histocompatibility at [symbol] | IPI00472073, | 40,566.20 | | 0 | 0 | 3 | 0 | 0 | 0 | 3 |
| | IPI00472767 | | | | | | | | | |
| HLA-C; HLA-B; MICA; LOC730410 Isoform 2 of HLA class I histoco [symbol] | IPI00472035, | 36,779.50 | | 0 | 4 | 2 | 0 | 0 | 0 | 2 |
| | IPI00745649 | | | | | | | | | |
| HMCN1 Hemicentin | IPI00045512, | 613,665.50 | Y | 0 | 0 | 2 | 0 | 0 | 0 | 2 |
| | IPI00549757, | | | | | | | | | |
| | IPI00746225 | | | | | | | | | |
| HP Haptoglobin precursor | IPI00641737 | 46,704.70 | | 16 | 0 | 49 | 43 | 56 | 49 | 221 |
| HPR Isoform 1 of Haptoglobin-related protein precursor | IPI00477597, | 38,989.50 | | 0 | 0 | 0 | 0 | 2 | 1 | 3 |
| | IPI00607707 | | | | | | | | | |
| HPX Hemopexin precursor | IPI00022488 | 51,658.50 | Y | 18 | 5 | 87 | 44 | 70 | 49 | 273 |
| HRG Histidine-rich glycoprotein precursor | IPI00022371 | 59,558.60 | Y | 8 | 4 | 22 | 35 | 2 | 0 | 69 |
| HSPG2 Basement membrane-specific heparan sulfate proteoglyc [symbol] | IPI00024284 | 468,787.50 | Y | 0 | 0 | 6 | 9 | 6 | 3 | 24 |
| HTRA1 Serine protease HTRA1 precursor | IPI00003176, | 51,269.30 | | 0 | 0 | 5 | 1 | 0 | 0 | 6 |
| | IPI00643586 | | | | | | | | | |
| Hypothetical protein | IPI00784807 | 51,305.90 | | 0 | 0 | 8 | 6 | 4 | 7 | 20 |
| Ig heavy chain V-III region BRO | IPI00382480 | 13,208.70 | | 0 | 0 | 6 | 0 | 0 | 0 | 6 |
| Ig heavy chain V-III region BUT | IPI00382481 | 12,358.80 | | 0 | 0 | 5 | 1 | 2 | 4 | 12 |
| Ig heavy chain V-III region CAM | IPI00382482 | 13,645.20 | | 0 | 0 | 7 | 4 | 0 | 8 | 11 |
| Ig heavy chain V-III region GAL | IPI00382500 | 12,708.10 | | 0 | 2 | 3 | 0 | 0 | 0 | 3 |
| Ig heavy chain V-III region JON | IPI00382499 | 12,543.60 | | 0 | 0 | 4 | 2 | 2 | 0 | 10 |
| Ig heavy chain V-III region NIE | IPI00382486 | 13,222.70 | | 0 | 0 | 0 | 2 | 0 | 0 | 2 |
| Ig heavy chain V-III region WAS | IPI00382493 | 13,071.50 | | 0 | 0 | 3 | 0 | 0 | 0 | 3 |
| Ig heavy chain V-III region WEA | IPI00382476 | 12,237.90 | | 0 | 0 | 2 | 0 | 0 | 0 | 2 |
| Ig kappa chain V-I region DEE | IPI00387025 | 11,642.30 | | 0 | 0 | 0 | 2 | 2 | 0 | 4 |
| Ig kappa chain V-I region EU | IPI00387026 | 11,770.50 | | 0 | 0 | 1 | 0 | 0 | 0 | 1 |
| Ig kappa chain V-I region Lay | IPI00387097 | 11,816.10 | | 0 | 0 | 1 | 0 | 0 | 0 | 1 |
| Ig kappa chain V-I region Mev | IPI00387105 | 11,852.30 | | 0 | 0 | 4 | 3 | 6 | 2 | 15 |
| Ig kappa chain V-I region Ni | IPI00387106 | 12,225.80 | | 0 | 0 | 8 | 5 | 4 | 4 | 21 |
| Ig kappa chain V-I region Scw | IPI00387101 | 11,746.20 | | 0 | 0 | 6 | 0 | 0 | 0 | 6 |
| Ig kappa chain V-I region Wes | IPI00003470 | 11,590.00 | | 0 | 0 | 2 | 1 | 0 | 0 | 3 |
| Ig kappa chain V-III region GOL | IPI00385252 | 11,812.80 | | 0 | 0 | 1 | 0 | 2 | 0 | 3 |
| Ig kappa chain V-III region IARC/BL41 precursor | IPI00386131 | 14,052.60 | | 0 | 0 | 4 | 1 | 1 | 2 | 8 |
| Ig kappa chain V-III region NG9 precursor (Fragment) | IPI00387116 | 10,711.10 | | 0 | 0 | 2 | 7 | 3 | 0 | 12 |
| Ig kappa chain V-III region SIE | IPI00387115 | 11,757.40 | | 0 | 0 | 8 | 6 | 6 | 2 | 22 |
| Ig lambda chain V-I region NEW | IPI00382421 | 11,434.80 | | 0 | 0 | 0 | 4 | 2 | 7 | 8 |
| Ig lambda chain V-II region TRO | IPI00382426 | 11,542.90 | | 0 | 0 | 3 | 0 | 0 | 0 | 3 |
| Ig lambda chain V-V region DEL | IPI00382442 | 11,324.00 | | 0 | 2 | 0 | 0 | 0 | 0 | 2 |
| IGF1 Insulin-like growth factor IA precursor | IPI00001610, | 17,008.10 | | 0 | 0 | 0 | 0 | 1 | 0 | 1 |
| | IPI00433029, | | | | | | | | | |
| | IPI00793994, | | | | | | | | | |
| | IPI00797681 | | | | | | | | | |
| IGF2 Isoform 1 of Insulin-like growth factor II precursor | IPI00001611, | 20,122.80 | | 0 | 0 | 4 | 0 | 0 | 0 | 4 |
| | IPI00215977, | | | | | | | | | |
| | IPI00657649 | | | | | | | | | |
| IGFAL5 Insulin-like growth factor- binding protein complex acid I | IPI00020996 | 66,020.60 | Y | 0 | 3 | 6 | 8 | 38 | 13 | 68 |
| IGFBP2 Insulin-like growth factor-binding protein 2 precursor | IPI00297284 | 35,119.10 | Y | 2 | 0 | 17 | 28 | 5 | 7 | 49 |
| IGFBP3 Insulin-like growth factor-binding protein 3 precursor | IPI00018305, | 31,656.10 | Y | 0 | 0 | 5 | 6 | 5 | 1 | 17 |
| IGFBP4 Insulin-like growth factor-binding protein 4 precursor | IPI00305380 | 27,915.70 | | 0 | 8 | 7 | 5 | 0 | 0 | 12 |
| IGFBP5 Insulin-like growth factor-binding protein 5 precursor | IPI00029236 | 30,552.00 | | 0 | 9 | 4 | 0 | 2 | 0 | 6 |
| IGFBP6 Insulin-like growth factor-binding protein 6 precursor | IPI00029235 | 25,303.80 | | 0 | 0 | 4 | 1 | 3 | 0 | 8 |
| IGFBP7 Insulin-like growth factor-binding protein 7 precursor | IPI00016915 | 29,111.80 | | 0 | 0 | 9 | 0 | 0 | 0 | 9 |
| IGHA2 Hypothetical protein DKFZp686C02220 (Fragment) | IPI00423461 | 54,140.90 | | 0 | 8 | 0 | 2 | 0 | 0 | 2 |
| IGHD IGHD protein | IPI00163446 | 62,949.30 | Y | 0 | 2 | 0 | 0 | 0 | 0 | 2 |
| IGHG1 Hypothetical protein DKFZp686P15220 | IPI00645363 | 51,705.90 | | 0 | 0 | 6 | 0 | 6 | 4 | 16 |
| IGHG1 IGHG1 protein | IPI00815926 | 51,696.40 | | 15 | 0 | 94 | 74 | 64 | 44 | 300 |
| IGHG1 IGHG1 protein | IPI00448925 | 60,083.40 | | 0 | 0 | 9 | 1 | 2 | 4 | 16 |
| IGHG1 IGHG1 protein | IPI00807531 | 51,967.90 | | 0 | 0 | 0 | 5 | 0 | 0 | 5 |
| IGHG1 IGHG1 protein | IPI00448938 | 51,376.20 | | 0 | 0 | 2 | 0 | 0 | 0 | 2 |
| IGHG3 IGHG3 protein | IPI00472345 | 57,136.70 | | 2 | 2 | 19 | 14 | 16 | 13 | 66 |
| IGHG4 IGHG4 protein | IPI00550640, | 51,967.30 | | 1 | 1 | 15 | 8 | 10 | 8 | 43 |
| | IPI00829814 | | | | | | | | | |
| IGHM IGHM protein | IPI00472610 | 52,647.50 | | 0 | 0 | 8 | 0 | 4 | 4 | 16 |
| IGHM IGHM protein | IPI00829768 | 68,106.50 | | 0 | 0 | 6 | 12 | 2 | 3 | 22 |
| IGHM IGHM protein | IPI00828205 | 65,020.50 | | 0 | 0 | 8 | 6 | 2 | 0 | 16 |
| IGHM IGHM protein | IPI00761159 | 52,567.20 | | 0 | 0 | 1 | 0 | 0 | 0 | 1 |
| IGHM IGHM protein | IPI00477090 | 67,276.80 | | 2 | 1 | 46 | 28 | 29 | 18 | 128 |
| IGJ immunoglobulin J chain | IPI00178926 | 18,080.50 | Y | 0 | 0 | 2 | 0 | 0 | 2 | 4 |
| IGKC IGKC protein | IPI00472961 | 25,918.40 | | 8 | 4 | 45 | 32 | 23 | 20 | 142 |
| IGKC IGKC protein | IPI00430808 | 25,628.40 | | 0 | 0 | 8 | 6 | 6 | 4 | 24 |
| IGKC IGKC protein | IPI00430847 | 25,689.00 | | 0 | 0 | 6 | 0 | 0 | 0 | 6 |
| IGKC IGKC protein | IPI00746963 | 25,585.00 | | 0 | 0 | 2 | 0 | 0 | 0 | 2 |
| IGKV1-5 IGKV1-5 protein | IPI00419424 | 26,216.60 | | 0 | 2 | 8 | 6 | 6 | 4 | 26 |
| IGKV4-1 Similar to Ig kappa chain V-IV region STH | IPI00026197, | 19,500.40 | | 1 | 0 | 12 | 9 | 7 | 6 | 35 |
| | IPI00386132 | | | | | | | | | |
| IGLV1-44 Anti-streptococcal/anti-myosin immunoglobulin lambd [symbol] | IPI00827522 | 11,575.90 | | 0 | 0 | 1 | 4 | 0 | 0 | 5 |
| ISLR Immunoglobulin superfamily containing leucine-rich repeat | IPI00023648 | 45,980.30 | | 0 | 0 | 2 | 9 | 0 | 0 | 2 |
| ITIH1 Inter-alpha-trypsin inhibitor heavy chain H1 precursor | IPI00292530 | 101,371.80 | Y | 0 | 6 | 47 | 13 | 25 | 19 | 110 |
| ITIH2 Inter-alpha-trypsin inhibitor heavy chain H2 precursor | IPI00305461 | 106,420.50 | Y | 3 | 2 | 55 | 40 | 23 | 23 | 146 |
| ITIH3 Inter-alpha-trypsin inhibitor heavy chain H3 precursor | IPI00028413 | 99,980.10 | Y | 0 | 0 | 0 | 0 | 0 | 1 | 1 |
| KLK10 Kallikrein-10 precursor | IPI00305266, | 30,119.60 | | 0 | 0 | 0 | 9 | 0 | 0 | 9 |
| | IPI00480121 | | | | | | | | | |
| KLK11 Isoform 1 of Kallikrein-11 precursor | IPI00002818, | 27,447.80 | | 0 | 0 | 2 | 0 | 3 | 0 | 5 |
| | IPI00218137 | | | | | | | | | |
| KLK6 Kallikrein-6 precursor | IPI00023845 | 26,837.60 | | 0 | 0 | 16 | 4 | 6 | 4 | 30 |
| KLK7 Isoform 1 of Kallikrein-7 precursor | IPI00028600 | 27,506.60 | | 0 | 0 | 0 | 1 | 0 | 0 | 1 |
| KLK9; KLK8 Isoform 1 of Neuropsin precursor | IPI00028484, | 28,029.40 | | 0 | 0 | 5 | 0 | 0 | 0 | 5 |
| | IPI00219892 | | | | | | | | | |
| KLKB1 Kallikrein B, plasma (Fletcher factor) 1 | IPI00654888, | 71,708.20 | | 0 | 3 | 18 | 5 | 12 | 27 | 62 |
| | IPI00783921 | | | | | | | | | |
| KNG1 Isoform LMW of Kininogen-1 precursor | IPI00215894, | 47,865.60 | Y | 10 | 2 | 50 | 35 | 36 | 34 | 171 |
| | IPI00797833 | | | | | | | | | |
| KRT1 Keratin, type II cytoskeletal 1 | IPI00220327 | 66,001.20 | Y | 0 | 3 | 18 | 39 | 26 | 21 | 107 |
| LBP Lipopolysaccharide-binding protein precursor | IPI00032311 | 53,368.00 | Y | 0 | 2 | 0 | 19 | 22 | 18 | 61 |
| LCAT Phosphatidylcholine-sterol acyltransferase precursor | IPI00022331 | 49,561.00 | Y | 0 | 0 | 0 | 0 | 2 | 0 | 2 |
| LCN2 Neutrophil gelatinase-associated lipocalin precursor | IPI00299547, | 22,570.90 | Y | 0 | 0 | 8 | 5 | 0 | 0 | 13 |
| | IPI00643623, | | | | | | | | | |
| | IPI00743064 | | | | | | | | | |
| LGALS1 Galectin-1 | IPI00219219 | 14,697.80 | | 2 | 0 | 5 | 1 | 0 | 0 | 8 |
| LRG1 Leucine-rich alpha-2-glycoprotein precursor | IPI00022417 | 38,161.70 | Y | 8 | 4 | 16 | 3 | 21 | 20 | 72 |
| LTBP1 Latent-transforming growth factor beta-binding protein, is [symbol] | IPI00220249, | 173,464.20 | Y | 0 | 0 | 2 | 8 | 0 | 0 | 2 |
| | IPI00302679, | | | | | | | | | |
| | IPI00410152, | | | | | | | | | |
| | IPI00784258 | | | | | | | | | |
| LUM Lumican precursor | IPI00020986 | 38,413.50 | Y | 2 | 4 | 8 | 2 | 9 | 9 | 34 |
| LYZ Lysozyme C precursor | IPI00019038 | 16,518.90 | | 0 | 0 | 0 | 6 | 2 | 2 | 10 |
| MMP2 72 kDa type IV collagenase precursor | IPI00027780 | 73,867.10 | Y | 0 | 0 | 9 | 4 | 5 | 2 | 20 |
| MSLN Isoform 2 of Mesothelin precursor | IPI00025110, | 68,054.00 | | 8 | 4 | 13 | 0 | 13 | 8 | 46 |
| | IPI00298690, | | | | | | | | | |
| | IPI00793522, | | | | | | | | | |
| | IPI00793649, | | | | | | | | | |
| | IPI00798210 | | | | | | | | | |
| MST1 Hepatocyte growth factor-like protein precursor | IPI00292218 | 80,359.90 | Y | 0 | 0 | 11 | 0 | 6 | 0 | 17 |
| Multi-functional protein MFP | IPI00828004 | 26,727.20 | | 0 | 0 | 0 | 2 | 0 | 0 | 2 |
| MXRA5 Matrix-remodelling-associated protein 5 precursor | IPI00012347 | 312,262.50 | Y | 0 | 0 | 21 | 0 | 5 | 1 | 27 |
| MYO6 Isoform 1 of Myosin-VI | IPI00069126, | 148,702.00 | | 0 | 0 | 0 | 0 | 0 | 2 | 2 |
| | IPI00816452, | | | | | | | | | |
| | IPI00816461 | | | | | | | | | |
| MYOC Myocilin precursor | IPI00019190 | 56,955.00 | Y | 0 | 0 | 2 | 0 | 0 | 0 | 2 |
| NID2 Nidogen-2 precursor | IPI00028908, | 151,376.50 | | 0 | 0 | 9 | 0 | 3 | 0 | 12 |
| | IPI00745450 | | | | | | | | | |
| NPC2 Epididymal secretory protein E1 precursor | IPI00301579 | 16,552.00 | | 0 | 0 | 5 | 4 | 3 | 0 | 12 |
| NUCB1 Nucleobindin-1 precursor | IPI00295542, | 53,861.60 | | 0 | 0 | 7 | 0 | 0 | 0 | 7 |
| | IPI00790899 | | | | | | | | | |
| ORM1 Alpha-1-acid glycoprotein 1 precursor | IPI00022429 | 23,494.10 | Y | 6 | 3 | 19 | 14 | 24 | 16 | 82 |
| ORM2 Alpha-1-acid glycoprotein 2 precursor | IPI00020091 | 23,585.20 | Y | 5 | 3 | 22 | 22 | 22 | 18 | 81 |
| PCOLCE Procollagen C-endopeptidase enhancer 1 precursor | IPI00299738 | 47,954.50 | Y | 0 | 0 | 6 | 8 | 0 | 0 | 14 |
| PEBP1 Phosphatidylethanolamine-binding protein 1 | IPI00219446 | 21,038.90 | | 0 | 2 | 0 | 2 | 0 | 2 | 6 |
| PGLYRP2 Isoform 1 of N-acetylmuramoyl-L-alanine amidase prec [symbol] | IPI00163207, | 62,199.90 | Y | 0 | 3 | 30 | 4 | 12 | 12 | 61 |
| | IPI00394992 | | | | | | | | | |
| PI16 protease inhibitor 16 precursor | IPI00301143 | 49,453.00 | Y | 0 | 0 | 3 | 0 | 0 | 0 | 3 |
| PLAUR Isoform 1 of Urokinase | IPI00010676, | 36,959.20 | | 0 | 0 | 2 | 0 | 0 | 0 | 2 |
| plasminogen activator surface rec | IPI00215706, | | | | | | | | | |
| | IPI00215707 | | | | | | | | | |
| PLEC1 Isoform 1 of Plectin-1 | IPI00014898, | 531,707.90 | | 0 | 2 | 1 | 0 | 0 | 0 | 3 |
| | IPI00186711, | | | | | | | | | |
| | IPI00398002, | | | | | | | | | |
| | IPI00398775, | | | | | | | | | |
| | IPI00398776, | | | | | | | | | |
| | IPI00398777, | | | | | | | | | |
| | IPI00398778, | | | | | | | | | |
| | IPI00398779, | | | | | | | | | |
| | IPI00420096 | | | | | | | | | |
| PLG Plasminogen precursor | IPI00019580 | 90,549.40 | Y | 8 | 7 | 131 | 78 | 107 | 78 | 409 |
| PLTP Isoform 1 of Phospholipid transfer protein precursor | IPI00643034 | 54,723.10 | | 0 | 2 | 10 | 6 | 15 | 6 | 39 |
| PON1 Serum paraoxonase/arylesterase 1 | IPI00218732 | 39,732.40 | Y | 0 | 3 | 2 | 0 | 7 | 4 | 16 |
| POSTN Isoform 1 of Periostin precursor | IPI00007960, | 93,300.00 | | 0 | 2 | 13 | 11 | 0 | 2 | 28 |
| | IPI00410241 | | | | | | | | | |
| PPBP; PPBPL2 Platelet basic protein precursor | IPI00022445 | 13,877.00 | Y | 0 | 0 | 0 | 2 | 0 | 0 | 2 |
| PRG4 Isoform C of Proteoglycan-4 precursor | IPI00655976 | 141,091.10 | | 0 | 3 | 9 | 12 | 0 | 0 | 24 |
| PROCR Endothelial protein C receptor precursor | IPI00009276 | 30,697.30 | | 0 | 0 | 2 | 0 | 0 | 0 | 2 |
| PROS1 Vitamin K-dependent protein S precursor | IPI00294004 | 75,105.40 | Y | 0 | 0 | 8 | 0 | 0 | 0 | 8 |
| PSAP Isoform Sap-mu-O of Proactivator polypeptide precursor | IPI00012503, | 58,094.00 | | 2 | 0 | 11 | 4 | 2 | 0 | 19 |
| | IPI00219825 | | | | | | | | | |
| PTGDS Prostaglandin-H2 D-isomerase precursor | IPI00013179, | 21,011.10 | Y | 3 | 2 | 11 | 9 | 0 | 3 | 34 |
| | IPI00513767 | | | | | | | | | |
| QSCN6 Isoform 1 of Sulfhydryl oxidase 1 precursor | IPI00003590, | 82,560.70 | Y | 0 | 2 | 2 | 1 | 12 | 4 | 21 |
| | IPI00465016 | | | | | | | | | |
| RBP4 Plasma retinol-binding protein precursor | IPI00022420, | 22,992.30 | Y | 5 | 0 | 36 | 3 [symbol] | 24 | 20 | 120 |
| | IPI00480192 | | | | | | | | | |
| REG1A Lithostathine 1 alpha precursor | IPI00009027 | 18,712.90 | | 0 | 0 | 0 | 0 | 2 | 0 | 2 |
| RNASE1 Ribonuclease pancreatic precursor | IPI00014048 | 17,625.80 | | 0 | 0 | 3 | 12 | 2 | 0 | 17 |
| RNASE2 Nonsecretory ribonuclease precursor | IPI00019449 | 18,335.70 | | 0 | 0 | 3 | 0 | 0 | 0 | 3 |
| RNASE4 Ribonuclease 4 precursor | IPI00029699 | 16,822.40 | | 0 | 0 | 0 | 0 | 3 | 2 | 7 |
| RNASET2 Isoform 1 of Ribonuclease T2 precursor | IPI00414896 | 29,463.20 | | 0 | 0 | 4 | 0 | 0 | 0 | 4 |
| SAA1; SAA2 Serum amyloid A protein precursor | IPI00552578 | 13,514.50 | | 0 | 0 | 8 | 1 | 0 | 0 | 15 |
| SAA4 Serum amyloid A-4 protein precursor | IPI00019399 | 14,789.30 | Y | 0 | 0 | 1 | 0 | 0 | 0 | 1 |
| SCGB1A1 Uteroglobin precursor | IPI00006705 | 9,976.20 | | 0 | 0 | 2 | 0 | 0 | 0 | 2 |
| SECTM1 Secreted and transmembrane protein 1 precursor | IPI00170635 | 27,020.50 | | 0 | 0 | 2 | 0 | 0 | 0 | 2 |
| SEPP1 Selenoprotein P precursor | IPI00029061, | 42,686.30 | Y | 0 | 0 | 2 | 0 | 2 | 0 | 4 |
| | IPI00798100 | | | | | | | | | |
| SERPINA1 Alpha-1-antitrypsin precursor | IPI00553177 | 46,719.90 | | 23 | 8 | 113 | 82 | 82 | 65 | 373 |
| SERPINA10 Protein Z-dependent protease inhibitor precursor | IPI00007199 | 55,097.60 | Y | 0 | 0 | 0 | 0 | 0 | 2 | 2 |
| SERPINA3 Isoform 1 of Alpha-1-antichymotrypsin precursor | IPI00550991 | 50,582.50 | | 8 | 2 | 50 | 16 | 32 | 33 | 146 |
| SERPINA4 Kallistatin precursor | IPI00328609 | 48,526.00 | Y | 0 | 0 | 10 | 21 | 23 | 18 | 72 |
| SERPINA6 Corticosteroid-binding globulin precursor | IPI00027482 | 45,124.10 | Y | 1 | 0 | 0 | 0 | 9 | 6 | 16 |
| SERPINA7 Thyroxine-binding globulin precursor | IPI00292946 | 46,307.60 | Y | 0 | 2 | 0 | 0 | 1 | 1 | 4 |
| SERPINC1 Antithrombin III variant | IPI00032179 | 52,675.10 | Y | 5 | 4 | 23 | 7 | 33 | 23 | 94 |
| SERPIND1 Heparin cofactor 2 precursor | IPI00292950 | 60,162.60 | Y | 0 | 0 | 28 | 9 | 25 | 21 | 83 |
| SERPINE1 Plasminogen activator inhibitor 1 precursor | IPI00007118 | 45,042.20 | | 0 | 0 | 0 | 0 | 3 | 0 | 3 |
| SERPINF1 Pigment epithelium-derived factor precursor | IPI00006114 | 46,326.40 | Y | 3 | 0 | 0 | 45 | 39 | 29 | 124 |
| SERPINF2 Alpha-2-antiplasmin precursor | IPI00029863 | 55,047.20 | Y | 0 | 0 | 12 | 5 | 11 | 9 | 37 |
| SERPING1 Plasma protease C1 inhibitor precursor | IPI00291866 | 55,137.50 | Y | 3 | 2 | 26 | 24 | 24 | 22 | 111 |
| SFRP4 Secreted frizzled-related protein 4 precursor | IPI00017557 | 39,808.80 | | 0 | 0 | 0 | 0 | 1 | 0 | 1 |
| SHBG Isoform 1 of Sex hormone-binding globulin precursor | IPI00023019 | 43,762.60 | Y | 0 | 3 | 26 | 1 | 18 | 12 | 60 |
| SOCS7 similar to Suppressor of cytokine signaling 7 | IPI00740805 | 64,171.20 | | 0 | 0 | 1 | 0 | 0 | 0 | 1 |
| SOD1 16 kDa protein | IPI00218733, | 16,103.70 | | 0 | 0 | 7 | 6 | 2 | 0 | 15 |
| | IPI00783680 | | | | | | | | | |
| SPARCL1 SPARC-like protein 1 precursor | IPI00296777 | 75,197.50 | | 0 | 0 | 0 | 4 | 0 | 0 | 4 |
| SPON1 Spondin-1 precursor | IPI00171473 | 90,955.60 | | 0 | 0 | 0 | 0 | 5 | 0 | 5 |
| SPP1 Isoform A of Osteopontin precursor | IPI00021000, | 35,404.60 | | 0 | 0 | 5 | 0 | 0 | 0 | 5 |
| | IPI00218874, | | | | | | | | | |
| | IPI00306339, | | | | | | | | | |
| | IPI00385896 | | | | | | | | | |
| SPP2 Secreted phosphoprotein 24 precursor | IPI00011832 | 24,320.00 | Y | 0 | 0 | 4 | 0 | 0 | 0 | 4 |
| SVEP1 polydom | IPI00301288, | 390,478.00 | | 0 | 0 | 4 | 0 | 0 | 0 | 4 |
| | IPI00719216 | | | | | | | | | |
| TAGLN2; CCDC19 Transgelin-2 | IPI00550363, | 22,373.90 | | 0 | 0 | 1 | 0 | 2 | 0 | 11 |
| | IPI00644531, | | | | | | | | | |
| | IPI00647915 | | | | | | | | | |
| TF Serotransferrin precursor | IPI00022463 | 77,032.20 | Y | 36 | 16 | 141 | 70 | 150 | 112 | 525 |
| TGFBI Transforming growth factor-beta-induced protein ig-h3 pr [symbol] | IPI00018219 | 74,664.90 | Y | 0 | 1 | 12 | 0 | 5 | 10 | 28 |
| THBS1 Thrombospondin-1 precursor | IPI00296099 | 129,363.70 | | 2 | 0 | 8 | 4 | 4 | 2 | 20 |
| THY1 Thy-1 membrane glycoprotein precursor | IPI00022892, | 17,917.20 | | 0 | 0 | 4 | 0 | 0 | 0 | 4 |
| | IPI00555577 | | | | | | | | | |
| TIMP1 Metalloproteinase inhibitor 1 precursor | IPI00032292 | 23,153.10 | Y | 4 | 7 | 18 | 10 | 6 | 11 | 59 |
| TIMP2 Metalloproteinase inhibitor 2 precursor | IPI00027166, | 24,381.90 | | 0 | 0 | 2 | 0 | 2 | 0 | 4 |
| | IPI00787781, | | | | | | | | | |
| | IPI00788747 | | | | | | | | | |
| TMEM110; ITIH4 Isoform 1 of Inter-alpha-trypsin inhibitor heavy | IPI00294193, | 103,308.40 | Y | 12 | 9 | 48 | 47 | 69 | 99 | 244 |
| | IPI00790993 | | | | | | | | | |
| TTR Transthyretin precursor | IPI00022432 | 15,868.90 | Y | 10 | 3 | 32 | 31 | 20 | 18 | 114 |
| VASN Vasorin precursor | IPI00395488 | 71,696.10 | | 0 | 2 | 2 | 0 | 0 | 0 | 4 |
| VH3 protein (Fragment) | IPI00383732 | 15,750.60 | | 0 | 0 | 2 | 0 | 0 | 0 | 2 |
| VMO1 Vitelline membrane outer layer protein1 homolog precurs [symbol] | IPI00216914 | 21,516.20 | | 0 | 0 | 2 | 0 | 0 | 0 | 2 |
| VSIG4 Isoform 1 of V-set and immunoglobulin domain-containin [symbol] | IPI00027038, | 43,969.00 | | 0 | 0 | 2 | 0 | 0 | 0 | 2 |
| | IPI00552123 | | | | | | | | | |
| VTN Vitronectin precursor | IPI00298971 | 54,288.10 | Y | 3 | 3 | 15 | 16 | 19 | 14 | 70 |
| VWF von Willebrand factor precursor | IPI00023014, | 309,267.50 | Y | 0 | 0 | 2 | 4 | 0 | 0 | 6 |
| | IPI00788786 | | | | | | | | | |
| WFDC2 Isoform 1 of WAP four-disulfide core domain protein 2 p [symbol] | IPI00291488 | 12,974.40 | | 3 | 0 | 7 | 6 | 2 | 0 | 18 |

[symbol]

indicates data missing or illegible when filed

TABLE 4
Common extracellular and membranous ascites proteins identified within the supernatant of four ovarian cancer cell lines.
# of Unique Peptides shown correspond to the number of unique peptides identified within ascites fluid

| | | Protein | | | | | | | | |
|---|---|---|---|---|---|---|---|---|---|---|
| | Protein | Molecular | | | | | | | | Total |
| | Accession | Weight | Plasma | # Unique Peptides | | | | | | Unique |
| Protein Name | Numbers | (AMU) | Proteoma | 50K | 100K | NH4HCO3 | NH4HCO3#2 | PO4SO4 | PO4SO4#2 | Peptides |
| A2M Alpha-2-macroglobulin | IPI00478003 | 163,258.80 | | 15 | 8 | 69 | 26 | 85 | 53 | 256 |
| precursor | | | | | | | | | | |
| AGRN Agrin precursor | IPI00374563, | 214,820.00 | | 0 | 0 | 8 | 7 | 3 | 0 | 18 |
| | IPI00795766 | | | | | | | | | |
| AMBP AMBP protein precursor | IPI00022426 | 38,981.50 | Y | 5 | 5 | 31 | 14 | 15 | 14 | 84 |
| BCAM Lutheran blood group | IPI00794214 | 61,041.90 | | 0 | 0 | 4 | 0 | 0 | 0 | 4 |
| glycoprotein precursor | | | | | | | | | | |
| C14orf141; LTBP2 | IPI00292150 | 195,038.50 | | 0 | 0 | 2 | 4 | 0 | 0 | 6 |
| Latent-transforming | | | | | | | | | | |
| growth factor beta-bindi | | | | | | | | | | |
| CD248 Isoform 1 of Endosialin | IPI00006971 | 80,839.70 | | 0 | 0 | 2 | 0 | 0 | 0 | 2 |
| precursor | | | | | | | | | | |
| CD59 CD59 glycoprotein | IPI00011302 | 14,159.20 | | 0 | 0 | 2 | 0 | 0 | 0 | 2 |
| precursor | | | | | | | | | | |
| CLU Clusterin precursor | IPI00291262, | 52,476.90 | Y | 0 | 4 | 12 | 14 | 4 | 11 | 45 |
| | IPI00400826, | | | | | | | | | |
| | IPI00795633 | | | | | | | | | |
| COMP 80 kDa protein | IPI00643348 | 79,676.20 | | 0 | 0 | 0 | 0 | 0 | 2 | 2 |
| CPA4 Carboxypeptidase A4 | IPI00008894 | 47,334.40 | | 0 | 0 | 4 | 0 | 0 | 0 | 4 |
| precursor | | | | | | | | | | |
| CST3 Cystatin-C precursor | IPI00032293 | 15,781.20 | Y | 3 | 2 | 0 | 14 | 0 | 0 | 19 |
| CST6 Cystatin-M precursor | IPI00019954, | 16,493.10 | | 0 | 0 | 2 | 0 | 0 | 0 | 2 |
| | IPI00788184 | | | | | | | | | |
| CTGF Isoform 1 of Connective | IPI00020977, | 38,072.70 | | 0 | 0 | 0 | 2 | 0 | 0 | 2 |
| tissue growth factor precursor | IPI00220647 | | | | | | | | | |
| DAG1 Dystroglycan precursor | IPI00028911 | 97,563 30 | Y | 0 | 0 | 5 | 0 | 0 | 0 | 5 |
| DKK3 Dickkopf-related | IPI00002714, | 38,272.30 | | 0 | 0 | 2 | 0 | 0 | 0 | 2 |
| protein 3 precursor | IPI00383937 | | | | | | | | | |
| DSC2 Isoform 2A of | IPI00025846, | 99,944.60 | | 0 | 0 | 2 | 0 | 0 | 0 | 2 |
| Desmocollin-2 precursor | IPI00220146 | | | | | | | | | |
| DSG2 desmoglein 2 | IPI00028931 | 122,276.40 | Y | 0 | 0 | 8 | 0 | 0 | 0 | 8 |
| preproprotein | | | | | | | | | | |
| ECM1 Extracellular matrix | IPI00003351, | 60,655.40 | Y | 0 | 2 | 16 | 0 | 25 | 17 | 60 |
| protein 1 precursor | IPI00645849 | | | | | | | | | |
| EFEMP1 Isoform 1 of | IPI00029658, | 54,621.10 | | 0 | 2 | 28 | 6 | 25 | 16 | 78 |
| EGF-containing fibulin-like | IPI00220813, | | | | | | | | | |
| extracellular [symbol] | IPI00220814, | | | | | | | | | |
| | IPI00220815 | | | | | | | | | |
| FAM3C Protein FAM3C | IPI00021923 | 24,662.90 | | 0 | 0 | 6 | 5 | 2 | 2 | 15 |
| precursor | | | | | | | | | | |
| FBLN1 Isoform C of | IPI00296537 | 74,441.90 | Y | 0 | 0 | 3 | 0 | 5 | 2 | 10 |
| Fibulin-1 precursor | | | | | | | | | | |
| FBLN1 Isoform D of | IPI00296534 | 77,240.80 | Y | 0 | 0 | 34 | 0 | 29 | 28 | 91 |
| Fibulin-1 precursor | | | | | | | | | | |
| FOLR1 Folate receptor | IPI00441498 | 29,800.50 | | 0 | 0 | 4 | 0 | 0 | 0 | 4 |
| alpha precursor | | | | | | | | | | |
| FSTL1 Follistatin-related | IPI00029723 | 34,967.30 | | 0 | 0 | 4 | 0 | 0 | 0 | 4 |
| protein 1 precursor | | | | | | | | | | |
| GAPDH | IPI00219018 | 36,035.30 | Y | 5 | 3 | 14 | 10 | 0 | 0 | 32 |
| Glyceraldehyde-3-phosphate | | | | | | | | | | |
| dehydrogenase | | | | | | | | | | |
| GLOD4 Uncharacterized | IPI00007102, | 54,995.30 | | 0 | 0 | 2 | 0 | 0 | 0 | 2 |
| protein C17orf25 | IPI00032575, | | | | | | | | | |
| | IPI00745272 | | | | | | | | | |
| GM2A Ganglioside GM2 | IPI00018236 | 20,804.90 | | 0 | 0 | 3 | 0 | 0 | 0 | 3 |
| activator precursor | | | | | | | | | | |
| GOLPH2 Golgi | IPI00171411, | 46,254.70 | | 0 | 0 | 1 | 0 | 0 | 0 | 1 |
| phosphoprotein 2 | IPI00759659, | | | | | | | | | |
| | IPI00784293 | | | | | | | | | |
| GPX3 Glutathione peroxidase | IPI00026199 | 25,488.20 | Y | 2 | 2 | 7 | 9 | 0 | 0 | 20 |
| 3 precursor | | | | | | | | | | |
| GSN Isoform 1 of Gelsolin | IPI00026314 | 85,679.80 | Y | 6 | 4 | 57 | 41 | 39 | 37 | 184 |
| precursor | | | | | | | | | | |
| HSPG2 Basement | IPI00024284 | 468,787.50 | Y | 0 | 0 | 6 | 9 | 6 | 3 | 24 |
| membrane-specific | | | | | | | | | | |
| heparan sulfate proteogly | | | | | | | | | | |
| HTRA1 Serine protease | IPI00003176, | 51,269.30 | | 0 | 0 | 5 | 1 | 0 | 0 | 6 |
| HTRA1 precursor | IPI00643586 | | | | | | | | | |
| IGFBP2 Insulin-like growth | IPI00297284 | 35,119.10 | Y | 2 | 3 | 17 | 20 | 5 | 2 | 49 |
| factor-binding protein | | | | | | | | | | |
| 2 precursor | | | | | | | | | | |
| IGFBP3 Insulin-like growth | IPI00018305, | 31,656.10 | Y | 0 | 0 | 5 | 6 | 5 | 1 | 17 |
| factor-binding protein | IPI00556155 | | | | | | | | | |
| 3 precursor | | | | | | | | | | |
| IGFBP4 Insulin-like growth | IPI00305380 | 27,915.70 | | 0 | 0 | 7 | 5 | 0 | 0 | 12 |
| factor-binding protein | | | | | | | | | | |
| 4 precursor | | | | | | | | | | |
| IGFBP5 Insulin-like growth | IPI00029236 | 30,552.00 | | 0 | 0 | 4 | 0 | 2 | 0 | 6 |
| factor-binding protein | | | | | | | | | | |
| 5 precursor | | | | | | | | | | |
| IGFBP6 Insulin-like growth | IPI00029235 | 25,303.80 | | 0 | 0 | 4 | 1 | 3 | 0 | 8 |
| factor-binding protein | | | | | | | | | | |
| 6 precursor | | | | | | | | | | |
| IGFBP7 Insulin-like growth | IPI00016915 | 29,111.80 | | 0 | 0 | 9 | 0 | 0 | 0 | 9 |
| factor-binding protein | | | | | | | | | | |
| 7 precursor | | | | | | | | | | |
| KLK6 Kallikrein-6 precursor | IPI00023845 | 26,837.60 | | 0 | 0 | 16 | 4 | 6 | 4 | 30 |
| KLK9; KLK8 Isoform 1 of | IPI00028484, | 28,029.40 | | 0 | 0 | 5 | 0 | 0 | 0 | 5 |
| Neuropsin precursor | IPI00219892 | | | | | | | | | |
| LCN2 Neutrophil | IPI00299547, | 22,570.90 | Y | 0 | 0 | 8 | 5 | 0 | 0 | 13 |
| gelatinase-associated | IPI00643623, | | | | | | | | | |
| lipocalin precursor | IPI00743064 | | | | | | | | | |
| LGALS1 Galectin-1 | IPI00219219 | 14,697.80 | | 2 | 0 | 5 | 1 | 0 | 0 | 8 |
| LRG1 Leucine-rich | IPI00022417 | 38,161.70 | Y | 8 | 4 | 16 | 3 | 21 | 20 | 72 |
| alpha-2-glycoprotein precursor | | | | | | | | | | |
| MST1 Hepatocyte growth | IPI00292218 | 80,359.90 | Y | 0 | 0 | 11 | 0 | 6 | 0 | 17 |
| factor-like protein precursor | | | | | | | | | | |
| MXRA5 | IPI00012347 | 312,262.50 | Y | 0 | 0 | 21 | 0 | 5 | 1 | 27 |
| Matrix-remodelling-associated | | | | | | | | | | |
| protein 5 precursor | | | | | | | | | | |
| NID2 Nidogen-2 precursor | IPI00028908, | 151,376.50 | | 0 | 0 | 9 | 0 | 3 | 0 | 12 |
| | IPI00745450 | | | | | | | | | |
| NPC2 Epididymal secretory | IPI00301579 | 16,552.00 | | 0 | 0 | 5 | 4 | 3 | 0 | 12 |
| protein E1 precursor | | | | | | | | | | |
| NUCB1 Nucleobindin-1 | IPI00295542, | 53,861.60 | | 0 | 0 | 7 | 0 | 0 | 0 | 7 |
| precursor | IPI00790899 | | | | | | | | | |
| PCOLCE Procollagen | IPI00299738 | 47,954.50 | Y | 0 | 0 | 6 | 8 | 0 | 0 | 14 |
| C-endopeptidase enhancer | | | | | | | | | | |
| 1 precursor | | | | | | | | | | |
| PEBP1 | IPI00219446 | 21,038.90 | | 0 | 2 | 0 | 2 | 0 | 2 | 6 |
| Phosphatidylethanolamine- | | | | | | | | | | |
| binding protein 1 | | | | | | | | | | |
| PLAUR Isoform 1 of | IPI0010676, | 36,959.20 | | 0 | 0 | 2 | 0 | 0 | 0 | 2 |
| Urokinase plasminogen | IPI00215706, | | | | | | | | | |
| activator surface re | IPI00215707 | | | | | | | | | |
| PLEC1 Isoform 1 of Plectin-1 | IPI00014898, | 531,707.90 | | 0 | 2 | 1 | 0 | 0 | 0 | 3 |
| | IPI00186711, | | | | | | | | | |
| | IPI00398002, | | | | | | | | | |
| | IPI00398775, | | | | | | | | | |
| | IPI00398776, | | | | | | | | | |
| | IPI00398777, | | | | | | | | | |
| | IPI00398778, | | | | | | | | | |
| | IPI00398779, | | | | | | | | | |
| | IPI00420096 | | | | | | | | | |
| PLTP Isoform 1 of | IPI00643034 | 54,723.10 | | 0 | 2 | 10 | 6 | 15 | 6 | 39 |
| Phospholipid transfer | | | | | | | | | | |
| protein precursor | | | | | | | | | | |
| PROCR Endothelial protein | IPI00009276 | 30,697.30 | | 0 | 0 | 2 | 0 | 0 | 0 | 2 |
| C receptor precursor | | | | | | | | | | |
| PROS1 Vitamin K-dependent | IPI00294004 | 75,105.40 | Y | 0 | 0 | 8 | 0 | 0 | 0 | 8 |
| protein S precursor | | | | | | | | | | |
| PSAP Isoform Sap-mu-0 | IPI00012503, | 58,094.00 | | 2 | 0 | 11 | 4 | 2 | 0 | 19 |
| of Proactivator | IPI00219825 | | | | | | | | | |
| polypeptide precursor | | | | | | | | | | |
| QSCN6 Isoform 1 of | IPI00003590, | 82,560.70 | Y | 0 | 2 | 2 | 1 | 12 | 4 | 21 |
| Sulfhydryl oxidase 1 precursor | IPI00465016 | | | | | | | | | |
| SECTM1 Secreted and | IPI00170635 | 27,020.50 | | 0 | 0 | 2 | 0 | 0 | 0 | 2 |
| transmembrane protein | | | | | | | | | | |
| 1 precursor | | | | | | | | | | |
| SERPINA6 | IPI00027482 | 45,124.10 | Y | 1 | 0 | 0 | 0 | 9 | 6 | 16 |
| Corticosteroid-binding | | | | | | | | | | |
| globulin precursor | | | | | | | | | | |
| SERPINE1 Plasminogen | IPI00007118 | 45,042.20 | | 0 | 0 | 0 | 0 | 3 | 0 | 3 |
| activator inhibitor 1 precursor | | | | | | | | | | |
| SERPINF1 Pigment | IPI00006114 | 46,326.40 | Y | 3 | 2 | 6 | 45 | 39 | 29 | 124 |
| epithelium-derived factor | | | | | | | | | | |
| precursor | | | | | | | | | | |
| SERPING1 Plasma protease | IPI00291866 | 55,137.50 | Y | 3 | 2 | 26 | 34 | 24 | 22 | 111 |
| C1 inhibitor precursor | | | | | | | | | | |
| SOD1 16 kDa protein | IPI00218733, | 16,103.70 | | 0 | 0 | 7 | 6 | 2 | 0 | 15 |
| | IPI00783680 | | | | | | | | | |
| SPP1 Isoform A of | IPI00021000, | 35,404.60 | | 0 | 0 | 5 | 0 | 0 | 0 | 5 |
| Osteopontin precursor | IPI00218874, | | | | | | | | | |
| | IPI00306339, | | | | | | | | | |
| | IPI00385896 | | | | | | | | | |
| SVEP1 polydom | IPI00301288, | 390,478.00 | | 0 | 0 | 4 | 0 | 0 | 0 | 4 |
| | IPI00719216 | | | | | | | | | |
| TAGLN2; CCDC19 | IPI00550363, | 22,373.90 | | 0 | 0 | 1 | 8 | 2 | 0 | 11 |
| Transgetin-2 | IPI00644531, | | | | | | | | | |
| | IPI00647915 | | | | | | | | | |
| TGFBI Transforming growth | IPI00018219 | 74,664.90 | Y | 0 | 1 | 12 | 0 | 5 | 10 | 18 |
| factor-beta-induced | | | | | | | | | | |
| protein Ig-h3 [symbol] | | | | | | | | | | |
| THBS1 Thrombospondin-1 | IPI00296099 | 129,363.70 | Y | 2 | 0 | 8 | 4 | 4 | 2 | 20 |
| precursor | | | | | | | | | | |
| TIMP1 Metalloproteinase | IPI00032292 | 23,153.10 | Y | 4 | 2 | 18 | 18 | 6 | 11 | 59 |
| inhibitor 1 precursor | | | | | | | | | | |
| TIMP2 Metalloproteinase | IPI00027166, | 24,381.90 | | 0 | 0 | 2 | 0 | 2 | 0 | 4 |
| inhibitor 2 precursor | IPI00787781, | | | | | | | | | |
| | IPI00788747 | | | | | | | | | |
| VASN Vasorin precursor | IPI00395488 | 71,696.10 | | 0 | 2 | 2 | 0 | 0 | 0 | 4 |
| VTN Vitronectin precursor | IPI00298971 | 54,288.10 | Y | 3 | 3 | 15 | 16 | 19 | 14 | 70 |
| WFDC2 Isoform 1 of WAP | IPI00291488 | 12,974.40 | | 3 | 0 | 7 | 6 | 2 | 0 | 18 |
| four-disulfide core domain | | | | | | | | | | |
| protein 2 | | | | | | | | | | |

[symbol]

indicates data missing or illegible when filed

TABLE 5
Panel of 52 Putative Ovarian Cancer Biomarkers
Proteins that were found by the Kislinger's group¹⁷, ovarian cancer cell lines or plasma proteome are designated with an Y

| | | Protein | | Ovarian | | | | | | | | |
|---|---|---|---|---|---|---|---|---|---|---|---|---|
| | Protein | Molecular | | Cancer | | # Unique Peptides | | | | | | Total |
| | Accession | Weight | | Cell | Plasma | | | | NH4HCO3 | | PO4SO4 | Unique |
| Protein Name | Numbers | (AMU) | [symbol] | Lines | Proteome | 50K | 100K | NH4HCO3 | #2 | PO4SO4 | #2 | Peptides |
| AGRM Agrin | IPI00374563, | 214,820.00 | Y | Y | | 0 | 0 | 0 | 7 | 3 | 0 | 18 |
| precursor | IPI00795766 | | | | | | | | | | | |
| BCAM Lutheran blood | IPI00794214 | 61,041.90 | Y | Y | | 0 | 0 | 4 | 0 | 0 | 0 | 4 |
| group glycoprotein | | | | | | | | | | | | |
| precursor | | | | | | | | | | | | |
| C14orf141; LTBP2 | IPI00292150 | 195,038.50 | | Y | | 0 | 0 | 2 | 4 | 0 | 0 | 6 |
| Latent-transforming | | | | | | | | | | | | |
| growth factor | | | | | | | | | | | | |
| beta-binding protein | | | | | | | | | | | | |
| 2 precursor | | | | | | | | | | | | |
| CD248 Isoform 1 of | IPI00006971 | 80,839.70 | | Y | | 0 | 0 | 2 | 0 | 0 | 0 | 2 |
| Endosialin precursor | | | | | | | | | | | | |
| CD59 CD59 | IPI00011302 | 14,159.20 | Y | Y | | 0 | 4 | 2 | 0 | 0 | 0 | 2 |
| glycoprotein | | | | | | | | | | | | |
| precursor | | | | | | | | | | | | |
| CLU Clusterin | IPI00291262, | 52,476.90 | Y | Y | Y | 0 | 4 | 12 | 14 | 4 | 11 | 45 |
| precursor | IPI00400826, | | | | | | | | | | | |
| | IPI00795633 | | | | | | | | | | | |
| CDMP 80 kDa protein | IPI00643348 | 79,676.20 | | Y | | 0 | 0 | 0 | 0 | 0 | 2 | 2 |
| CPA4 | IPI00008894 | 47,334.40 | | Y | | 0 | 0 | 4 | 0 | 0 | 0 | 4 |
| Carboxypeptidase | | | | | | | | | | | | |
| A4 precursor | | | | | | | | | | | | |
| CST3 Cystatin-C | IPI00032293 | 15,781.20 | Y | Y | Y | 2 | 0 | 0 | 14 | 0 | 0 | 19 |
| precursor | | | | | | | | | | | | |
| CST6 Cystatin-M | IPI00019954, | 16,493.10 | | Y | | 0 | 0 | 2 | 0 | 0 | 0 | 2 |
| precursor | IPI00788184 | | | | | | | | | | | |
| CTGF Isoform 1 | IPI00020977, | 38,072.70 | | Y | | 0 | 0 | 0 | 2 | 0 | 0 | 2 |
| of Connective tissue | IPI00220647 | | | | | | | | | | | |
| growth factor | | | | | | | | | | | | |
| precursor | | | | | | | | | | | | |
| DAG1 Dystroglycan | IPI00028911 | 97,563.30 | | Y | Y | 0 | 0 | 5 | 0 | 0 | 0 | 5 |
| precursor | | | | | | | | | | | | |
| DKK3 | IPI00002714, | 38,272.30 | | Y | | 0 | 0 | 2 | 0 | 0 | 0 | 2 |
| Dickkopf-related | IPI00383937 | | | | | | | | | | | |
| protein 3 precursor | | | | | | | | | | | | |
| DSC2 Isoform 2A | IPI00025846, | 99,944.60 | Y | Y | | 0 | 0 | 2 | 0 | 0 | 0 | 2 |
| of Desmocollin-2 | IPI00220146 | | | | | | | | | | | |
| precursor | | | | | | | | | | | | |
| DSG2 desmoglein 2 | IPI00028931 | 122,276.40 | Y | Y | Y | 0 | 0 | 8 | 0 | 0 | 0 | 8 |
| preproprotein | | | | | | | | | | | | |
| ECM1 Extracellular | IPI00003351, | 60,655.40 | Y | Y | Y | 0 | 2 | 16 | 0 | 25 | 17 | 60 |
| matrix protein 1 | IPI00645849 | | | | | | | | | | | |
| precursor | | | | | | | | | | | | |
| EFEMP1 Isoform 1 | IPI00029658, | 54,621.10 | Y | Y | | 0 | 1 | 28 | 6 | 25 | 16 | 78 |
| of EGF-containing | IPI00220813, | | | | | | | | | | | |
| fibulin-like | IPI00220814, | | | | | | | | | | | |
| extracellular matrix | IPI00220815 | | | | | | | | | | | |
| protein 1 precurs [symbol] | | | | | | | | | | | | |
| FAM3C Protein | IPI00021923 | 24,662.90 | Y | Y | | 0 | 0 | 6 | 5 | 2 | 2 | 15 |
| FAM3C precursor | | | | | | | | | | | | |
| FBLN1 Isoform C of | IPI00296537 | 74,441.90 | Y | Y | Y | 0 | 0 | 3 | 0 | 5 | 2 | 10 |
| Fibulin-1 precursor | | | | | | | | | | | | |
| FDLR1 Folate receptor | IPI00441498 | 29,800.50 | Y | Y | | 0 | 0 | 4 | 0 | 0 | 0 | 4 |
| alpha precursor | | | | | | | | | | | | |
| FSTL1 | IPI00029723 | 34,967.30 | | Y | | 0 | 0 | 4 | 0 | 0 | 0 | 4 |
| Follistatin-related | | | | | | | | | | | | |
| protein 1 precursor | | | | | | | | | | | | |
| GLOD4 | IPI00007102, | 54,995.30 | Y | Y | | 0 | 0 | 2 | 0 | 0 | 0 | 2 |
| Uncharacterized | IPI00032575, | | | | | | | | | | | |
| protein C17orf25 | IPI00745272 | | | | | | | | | | | |
| GM2A Ganglioside | IPI00018236 | 20,804.90 | | Y | | 0 | 0 | 3 | 0 | 0 | 0 | 3 |
| GM2 activator | | | | | | | | | | | | |
| precursor | | | | | | | | | | | | |
| GPX3 Glutathione | IPI00026199 | 25,488.20 | Y | Y | Y | 2 | 2 | 7 | 9 | 0 | 0 | 20 |
| peroxidase 3 precursor | | | | | | | | | | | | |
| HSPG2 Basement | IPI00024284 | 468,787.50 | Y | Y | Y | 0 | 4 | 6 | 9 | 6 | 3 | 24 |
| membrane-specific | | | | | | | | | | | | |
| heparan sulfate | | | | | | | | | | | | |
| proteoglycan protein | | | | | | | | | | | | |
| precursor | | | | | | | | | | | | |
| HTRA1 Serine | IPI00003176, | 51,269.30 | | Y | | 0 | 0 | 5 | 1 | 0 | 0 | 6 |
| protease | | | | | | | | | | | | |
| HTRA1 precursor | IPI00643586 | | | | | | | | | | | |
| IGFBP2 Insulin-like | IPI00297284 | 35,119.10 | | Y | Y | 2 | 3 | 17 | 20 | 5 | 2 | 49 |
| growth factor-binding | | | | | | | | | | | | |
| protein 2 precursor | | | | | | | | | | | | |
| IGFBP3 Insulin-like | IPI00018305, | 31,656.10 | | Y | Y | 0 | 2 | 9 | 0 | 5 | 1 | 17 |
| growth factor-binding | IPI00556155 | | | | | | | | | | | |
| protein 3 precursor | | | | | | | | | | | | |
| IGFBP4 Insulin-like | IPI00305380 | 27,915.70 | | Y | | 0 | 0 | 7 | 5 | 0 | 0 | 12 |
| growth factor-binding | | | | | | | | | | | | |
| protein 4 precursor | | | | | | | | | | | | |
| IGFBP5 Insulin-like | IPI00029236 | 30,552.00 | | Y | | 0 | 0 | 4 | 0 | 2 | 0 | 6 |
| growth factor-binding | | | | | | | | | | | | |
| protein 5 precursor | | | | | | | | | | | | |
| IGFBP6 Insulin-like | IPI00029235 | 25,303.80 | | Y | | 0 | 0 | 4 | 1 | 3 | 0 | 8 |
| growth factor-binding | | | | | | | | | | | | |
| protein 6 precursor | | | | | | | | | | | | |
| IGFBP7 Insulin-like | IPI00016915 | 29,111.80 | | Y | | 0 | 0 | 9 | 0 | 0 | 0 | 9 |
| growth factor-binding | | | | | | | | | | | | |
| protein 7 precursor | | | | | | | | | | | | |
| LRG1 Leucine-rich | IPI00022417 | 38,161.70 | Y | Y | Y | 8 | 4 | 16 | 3 | 21 | 20 | 72 |
| alpha-2-glycoprotein | | | | | | | | | | | | |
| precursor | | | | | | | | | | | | |
| MST1 Hepatocyte | IPI00292218 | 80,359.90 | | Y | Y | 0 | 0 | 11 | 0 | 6 | 0 | 17 |
| growth factor-like | | | | | | | | | | | | |
| protein precursor | | | | | | | | | | | | |
| MXRA5 | IPI00012347 | 312,262.50 | Y | Y | Y | 0 | 0 | 21 | 0 | 5 | 1 | 27 |
| Matrix-remodelling- | | | | | | | | | | | | |
| associated protein 5 | | | | | | | | | | | | |
| precursor (Adlican) | | | | | | | | | | | | |
| NID2 Nidogen-2 | IPI00028908, | 151,376.50 | Y | Y | | 0 | 0 | 9 | 0 | 3 | 0 | 12 |
| precursor | IPI00745450 | | | | | | | | | | | |
| NPC2 Epididymal | IPI00301579 | 16,552.00 | Y | Y | | 0 | 0 | 5 | 4 | 3 | 0 | 12 |
| secretory protein | | | | | | | | | | | | |
| E1 precursor | | | | | | | | | | | | |
| NUCB1 | IPI00295542, | 53,861.60 | Y | Y | | 0 | 0 | 7 | 0 | 0 | 0 | 7 |
| Nucleobindin-1 | IPI00790899 | | | | | | | | | | | |
| precursor | | | | | | | | | | | | |
| PCOLCE Procollagen | IPI00299738 | 47,954.50 | | Y | Y | 0 | 0 | 6 | 0 | 0 | 6 | 14 |
| C-endopeptidase | | | | | | | | | | | | |
| enhancer 1 precursor | | | | | | | | | | | | |
| PLEC1 Isoform 1 | IPI00014898, | 531,707.90 | Y | Y | | 0 | 4 | 1 | 0 | 0 | 0 | 3 |
| of Plectin-1 | IPI00186711, | | | | | | | | | | | |
| | IPI00398002, | | | | | | | | | | | |
| | IPI00398775, | | | | | | | | | | | |
| | IPI00398776, | | | | | | | | | | | |
| | IPI00398777, | | | | | | | | | | | |
| | IPI00398778, | | | | | | | | | | | |
| | IPI00398779, | | | | | | | | | | | |
| | IPI00420096 | | | | | | | | | | | |
| PLTP Isoform 1 of | IPI00643034 | 54,723.10 | Y | Y | | 0 | 2 | 10 | 0 | 19 | 6 | 39 |
| Phospholipid transfer | | | | | | | | | | | | |
| protein precursor | | | | | | | | | | | | |
| PROCR Endothelial | IPI00009276 | 30,697.30 | | Y | | 0 | 1 | 2 | 0 | 0 | 0 | 2 |
| protein C receptor | | | | | | | | | | | | |
| precursor | | | | | | | | | | | | |
| PROS1 Vitamin | IPI00294004 | 75,105.40 | Y | Y | Y | 0 | 0 | 8 | 0 | 0 | 0 | 8 |
| K-dependent protein | | | | | | | | | | | | |
| S precursor | | | | | | | | | | | | |
| PSAP Isoform | IPI00012503, | 58,094.00 | Y | Y | | 2 | 0 | 11 | 4 | 2 | 0 | 19 |
| Sap-mu-0 of | IPI00219825 | | | | | | | | | | | |
| Proactivator | | | | | | | | | | | | |
| polypeptide precursor | | | | | | | | | | | | |
| QSCN6 Isoform 1 | IPI00003590, | 82,560.70 | Y | Y | Y | 0 | 0 | 2 | 1 | 12 | 4 | 21 |
| of Sulfhydryl | IPI00465016 | | | | | | | | | | | |
| oxidase 1 precursor | | | | | | | | | | | | |
| SECTM1 Secreted and | IPI00170635 | 27,020.50 | | Y | | 0 | 0 | 2 | 0 | 0 | 0 | 2 |
| transmembrane | | | | | | | | | | | | |
| protein 1 precursor | | | | | | | | | | | | |
| SERPINA6 | IPI00027482 | 45,124.10 | Y | Y | Y | 1 | 0 | 6 | 0 | 9 | 0 | 16 |
| Corticosteroid-binding | | | | | | | | | | | | |
| globulin precursor | | | | | | | | | | | | |
| SOD1 16 kDa protein | IPI00218733, | 16,103.70 | Y | Y | | 0 | 0 | 7 | 6 | 2 | 0 | 15 |
| (Superoxide Desmutase 1) | IPI00783680 | | | | | | | | | | | |
| SVEP1 polydom | IPI00301288, | 390,478.00 | Y | Y | | 0 | 0 | 4 | 0 | 9 | 0 | 4 |
| (Sal-Ob) | IPI00719216 | | | | | | | | | | | |
| TAGLN2; CCDC19 | IPI00550363, | 22,373.90 | Y | Y | | 0 | 4 | 1 | 4 | 2 | 0 | 11 |
| Transgelin-2 | IPI00644531, | | | | | | | | | | | |
| | IPI00647915 | | | | | | | | | | | |
| TGFBI Transforming | IPI00018219 | 74,664.90 | Y | Y | Y | 0 | 1 | 12 | 0 | 5 | 10 | 28 |
| growth | | | | | | | | | | | | |
| factor-beta-induced | | | | | | | | | | | | |
| protein ig-h3 | | | | | | | | | | | | |
| precursor | | | | | | | | | | | | |
| VASN Vasorin | IPI00395488 | 71,696.10 | Y | Y | | 0 | 2 | 2 | 0 | 0 | 8 | 4 |
| precursor | | | | | | | | | | | | |

[symbol]

indicates data missing or illegible when filed

While the disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

(1) Jemal, A., Siegel, R., Ward, E., Hao, Y., Xu, J., Murray, T., Thun, M. J. (2008) Cancer statistics, 2008. CA Cancer J Clin 58, 71-96.
(2) Bhoola, S., Hoskins, W. J. (2006) Diagnosis and management of epithelial ovarian cancer. Obstet Gynecol 107, 1399-1410.
(3) Jacobs, I., Bast, R. C. J. (1989) The CA 125 tumour-associated antigen: a review of the literature. Hum Reprod 4, 1-12.
(4) Tuxen, M. K., Soletormos, G., Dombernowsky, P. (1995) Tumor markers in the management of patients with ovarian cancer. Cancer Treat Rev 21, 215-245.
(5) Bast, R. C. J., Knapp, R. C. (1985) Use of the CA 125 antigen in diagnosis and monitoring of ovarian carcinoma. Eur J Obstet Gynecol Reprod Biol 19, 354-356.
(6) Adam, R. A., Adam, Y. G. (2004) Malignant ascites: past, present, and future. J Am Coll Surg 198, 999-1011.
(7) Kassis, J., Klominek, J., Kohn, E. C. (2005) Tumor microenvironment: what can effusions teach us? Diagn Cytopathol 33, 316-319.
(8) Tamsma, J. T., Keizer, H. J., Meinders, A. E. (2001) Pathogenesis of malignant ascites: Starling's law of capillary hemodynamics revisited. Ann Oncol 12, 1353-1357.
(9) Hu, L., Hofmann, J., Zaloudek, C., Ferrara, N., Hamilton, T., Jaffe, R. B. (2002) Vascular endothelial growth factor immunoneutralization plus Paclitaxel markedly reduces tumor burden and ascites in athymic mouse model of ovarian cancer. Am J Pathol 161, 1917-1924.

(10) Good, D. M., Thongboonkerd, V., Novak, J., Bascands, J. L., Schanstra, J. P., Coon, J. J., Dominiczak, A., Mischak, H. (2007) Body fluid proteomics for biomarker discover: lessons from the past hold the key to success in the future. J Proteome Res 6, 4549-4555.
(11) Berchuck, A., Carney, M. (1997) Human ovarian cancer of the surface epithelium. Biochem Pharmacol 54, 541-544.
(12) Verheul, H. M., Hoekman, K., Jorna, A. S., Smit, E. F., Pinedo, H. M. (2000) Targeting vascular endothelial growth factor blockade: ascites and pleural effusion formation. Oncologist 5 Suppl 1, 45-50.
(13) Cho, C. K., Shan, S. J., Winsor, E. J., Diamandis, E. P. (2007) Proteomic analysis of human amniotic fluid. Mol Cell Proteomics 6, 1406-1415.
(14) Shaw, J. L., Smith, C. R., Diamandis, E. P. (2007) Proteomic analysis of human cervico-vaginal fluid. J Proteome Res 6, 2859-2865.
(15) de Souza, G. A., Godoy, L. M., Mann, M. (2006) Identification of 491 proteins in the tear fluid proteome reveals a large number of proteases and protease inhibitors. Genome Biol 7, R72.
(16) Pilch, B., Mann, M. (2006) Large-scale and high-confidence proteomic analysis of human seminal plasma. Genome Biol 7, R40.
(17) Gortzak-Uzan, L., Ignatchenko, A., Evangelou, A. I., Agochiya, M., Brown, K. A., Rosen, B., Shaw, P., Jurisica, I., Kislinger, T. (2008) A proteome resource of ovarian cancer ascites: integrated proteomic and bioinformatic analyses to identify putative biomarkers. J Proteome Res 7, 339-351.
(18) Petricoin, E. F., Paweletz, C. P., Liotta, L. A. (2002) Clinical applications of proteomics: Proteomic pattern diagnostics. J Mammary Gland Biol Neoplasia 7, 433-440.
(19) Anderson, N. L., Polanski, M., Pieper, R., Gatlin, T., Tirumalai, R. S., Conrads, T. P., Veenstra, T. D., Adam, R. A., Pounds, J. G., Fagan, R., Lobley, A. (2004) The human plasma proteome: a nonredundant list developed by combination of four separate sources. Mol Cell Proteomics 3, 311-326.
(20) Diamandis, E. P., Yousef, G. M., Soosaipillai, A., Grass, L., Porter, A., Little, S., Sotiropoulou, G. (2000) Immunofluorometric assay of human kallikrein 6 (zyme/protease M/neurosin) and preliminary clinical applications. Clin Biochem 33, 369-375.
(21) Keller, A., Nesvizhskii, A. I., Kolker, E., Aebersold, R. (2002) Empirical statistical model to estimate the accuracy of peptide identifications made by MS/MS and database search. Anal Chem 74, 5383-5392.
(22) Nesvizhskii, A. I., Keller, A., Kolker, E., Aebersold, R. (2003) A statistical model for identifying proteins by tandem mass spectrometry. Anal Chem 75, 4646-4658.
(23) Chou, K. C., Shen, H. B. (2007) Euk-mPLoc: a fusion classifier for large-scale eukaryotic protein subcellular location prediction by incorporating multiple sites. J Proteome Res 6, 1728-1734.
(24) Desrosiers, R. R., Beaulieu, E., Buchanan, M., Beliveau, R. (2007) Proteomic analysis of human proteins by two-dimensional gel electrophoresis and by antibody arrays following depletion of high-abundance proteins. Cell Biochem Biophys 49, 182-195.
(25) Shen, Y., Kim, J., Strittmatter, E. F., Jacobs, J. M., Camp, D. G. n., Fang, R., Tolie, N., Moore, R. J., Smith, R. D. (2005) Characterization of the human blood plasma proteome. Proteomics 5, 4034-4035.
(26) Echan, L. A., Tang, H. Y., Ali-Khan, N., Lee, K., Speicher, D. W. (2005) Depletion of multiple high-abundance proteins improves protein profiling capacities of human serum and plasma. Proteomics 5, 3292-3303.
(27) Anderson, N. L., Anderson, N. G. (2002) The human plasma proteome: history, character, and diagnostic prospects. Mol Cell Proteomics 1, 845-867.
(28) Ogata, Y., Charlesworth, M. C., Muddiman, D. C. (2005) Evaluation of protein depletion methods for the analysis of total-, phospho- and glycoproteins in lumbar cerebrospinal fluid. J Proteome Res 4, 837-845.
(29) Zolotarjova, N., Martosella, J., Nicol, G., Bailey, J., Boyse, B. E., Barrtt, W. C. (2005) Differences among techniques for high-abundant protein depletion. Proteomics 5, 3304-3313.
(30) Merrell, K., Southwick, K., Graves, S. W., Esplin, M. S., Lewis, N. E., Thulin, C. D. (2004) Analysis of low-abundance, low-molecular-weight serum proteins using mass spectrometry. J Biomol Tech 15, 238-248.
(31) Kulasingam, V., Diamandis, E. P. (2007) Proteomics analysis of conditioned media from three breast cancer cell lines: a mine for biomarkers and therapeutic targets. Mol Cell Proteomics 1997-2011.
(32) Faça, V. M., Ventura, A. P., Fitzgibbon, M. P., Pereira-Faca, S. R., Pitteri, S. J., Green, A. E., Ireton, R. C., Zhang, Q., Wang, H., O'Briant, K. C., Drescher, C. W., Schummer, M., McIntosh, M. W., Knudsen, B. S., Hanash, S. M. (2008) Proteomic analysis of ovarian cancer cells reveals dynamic processes of protein secretion and shedding of extra-cellular domains. PLoS ONE e2425.
(33) Nustad, K., Lebedin, Y., Lloyd, K. O., Shigemasa, K., de Bruijn, H. W., Jansson, B., Nilsson, O., Olsen, K. H., O'Brien, T. J. (2002) Epitopes on CA 125 from cervical mucus and ascites fluid and characterization of six new antibodies. Third report from the ISOBm TD-1 workshop. Tumour Biol 23, 303-314.
(34) Ohkubo, T., Yamamura, M., Kamiguchi, H., Sahashi, T., Hayashi, S., Shoitsuka, Y., Matsumoto, M., Kobayashi, K., Katsunuma, T. (1982) Alpha 1-antichymotrypsin and IgM as DNA-binding protein in serum—a possible diagnosis for malignant ovarian tumors. Tokai J Exp Clin Med 7, 325-329.
(35) Zhang, Z., Bast, R. C. J., Yu, Y., Sokoll, L. J., Rai, A. J., Rosenzweig, J., Cameron, B., Wang, Y. Y., Meng, X. Y., Berchuck, A., Van Haaften-Day, C., Hacker, N. F., de Bruijn, H. W., van der Zee, A. G., Jacobs, I. J., Fung, E. T., Chan, D. W. (2004) Three biomarkers identified from serum proteomic analysis for the detection of early stage ovarian cancer. Cancer Res 64, 5882-5890.
(36) Jackson, D., Craven, R. A., Hutson, R. C., Graze, I., Lueth, P., Tonge, R. P., Hartley, J. L., Nickson, J. A., Rayner, S. J., Johnston, C., Dieplinger, B., Hubalek, M., Wilkinson, N., Perren, T. J., Kohoe, S., Hall, G. D., Daxenbichler, G., Dieplinger, H., Selby, P. J., Banks, R. E. (2007) Proteomic profiling identifies afamin as a potential biomarker for ovarian cancer. Clin Cancer Res 13, 7370-7379.
(37) Diefenbach, C. S., Shah, Z., Iasonos, A., Barakat, R. R., Levine, D. A., Aghajanian, C., Sabbatini, P., Hensley, M. L., Konner, J., Tew, W., Spriggs, D., Fleisher, M., Thaler, H., Dupont, J. (2006) Preoperative serum YKL-40 is a marker for detection and prognosis of endometrial cancer. Gynecol Oncol 104, 435-442.
(38) Begum, F. D., Hogdall, C. K., Kjaer, S. K., Christensen, L., Blaakaer, J., Bock, J. E., Glud, E., Hoyer-Hansen, G., Ring-Larsen, H., Hogdall, E. V. (2004) The prognostic value of plasma soluble urokinase plasminogen activator receptor (suPAR) levels in stage III ovarian cancer patients. Anticancer Res 24, 1981-1985.
(39) Lundstrom, M. S., Hogdall, C. K., Nielsen, A. L., Nyholm, H. C. (2000) Serum tetranectin and CA125 in endometrial adenocarcinoma. Anticancer Res 20, 3903-3906.
(40) Luo, L. Y., Katsaros, D., Scorilas, A., Fracchioli, S., Bellino, R., van Gramberen, M., de Bruijn, H., Henrik, A., Stenman, U. H., Massobrio, M., van der Zee, A. G., Vergote, I., Diamandis, E. P. (2003) The serum concentration of human kallikrein 10 represents a novel biomarker for ovarian cancer diagnosis and prognosis. Cancer Res 63, 807-811.
(41) Rosen, D. G., Wang, L., Atkinson, J. N., Yu, Y., Lu, K. H., Diamandis, E. P., Hellstrom, I., Mok, S. C., Liu, J., Bast, R. C. J. (2005) Potential markers that complement expression of CA125 in epithelial ovarian cancer. Gynecol Oncol 99, 267-277.
(42) McIntosh, M. W., Liu, Y., Drescher, C., Urban, N., Diamandis, E. P. (2007) Validation and characterization of human kallikrein 11 as a serum marker for diagnosis of ovarian carcinoma. Clin Cancer Res 13, 4422-4428.
(43) Diamandis, E. P., Scorilas, A., Fracchioli, S., Van Gramberen, M., De Bruijn, H., Henrik, A., Soosaipillai, A., Grass, L., Yousef, G. M., Stenman, U. H., Massobrio, M., Van der Zee, A. G., Vergote, I., Katsaros, D. (2003) Human kallikrein 6 (hK6): a new potential serum biomarker for diagnosis and prognosis of ovarian cancer. J Clin Oncol 21, 1035-1043.
(44) Kishi, T., Soosaipillai, A., Grass, L., Little, S. P., Johnstone, E. M., Diamandis, E. P. (2004) Development of immunofluorometric assay and quantification of human kallikrein 7 in tissue extracts and biological fluids. Clin Chem 50, 709-716.
(45) Kishi, T., Grass, L., Soosaipillai, A., Scorilas, A., Harbeck, N., Schmalfeldt, B., Dorn, J., Mysliwiec, M., Schmitt, M., Diamandis, E. P. (2003) Human kallikrein 8, a novel biomarker for ovarian cancer. Cancer Res 63, 2771-2774.
(46) Lim, R., Ahmed, N., Borregaard, N., Riley, C., Wafai, R., Thompson, E. W., Quinn, M. A., Rice, G. E. (2007) Neutrophil gelatinase-associated lipocalin (NGAL) an early screening biomarker for ovarian cancer: NGAL is associated with epidermal growth factor-induced epithelio-mesenchymal transition. Int J Cancer 120, 2426-2434.
(47) Allen, H. J., Sucato, D., Woynarowska, B., Gottstine, S., Sharma, A., Bernacki, R. J. (1990) Role of galaptin in ovarian cancer adhesion to extracellular matrix in vitro. J Cell Biochem 43, 43-57.
(48) Allen, H. J., Sharma, A., Ahmed, H., Piver, M. S., Gamarra, M. (1993) Galaptin and galaptin-binding glycoconjugates in serum and effusions of carcinoma patients. Tumour Biol 14, 360-368.
(49) Demeter, A., Sziller, I., Csapo, Z., Olah, J., Keszler, G., Jeney, A., Papp, Z., Staub, M. (2005) Molecular prognostic markers in recurrent and in non-recurrent epithelial ovarian cancer. Anticancer Res 25, 2885-2889.
(50) Garzetti, G. G., Ciavattini, A., Lucarini, G., Goteri, G., de e Nictolis, M., Garbisa, S., Masiero, L., Romanini, C., Graziella, B. (1995) Tissue and serum metalloproteinase (MMP-2) expression in advanced ovarian serous cystoadenocarcinomas: clinical and prognostic implications. Anticancer Res 15, 2799-2804.
(51) Maatta, M., Talvensaari-Mattila, A., Turpeenniemi-Hujanen, T., Santala, M. (2007) Matrix metalloproteinase-2 (MMP-2) and -9 (MMP-9) and their tissue inhibitors (TIMP-1 and TIMP-2) in differential diagnosis between low and malignant potential (LMP) and malignant ovarian tumours. Anticancer Res 27, 2753-2758.
(52) Scholler, N., Gross, J. A., Garvik, B., Wells, L., Liu, Y., Loch, C. M., Ramirez, A. B., McIntosh, M. W., Lampe, P. D., Urban, N. (2008) Use of cancer-specific yeast-secreted in vivo biotinylated recombinant antibodies for serum biomarker discovery. J Transl Med 41.
(53) Pedersen, N., Schmitt, M., Ronne, E., Nicoletti, M. I., Hoyer-Hansen, G., Conese, M., Giavazzi, R., Dano, K., Kuhn, W., Janicke, F. (1993) A ligand-free, soluble urokinase receptor is present in the ascitic fluid from patients with ovarian cancer. J Clin Invest 92, 2160-2167.
(54) Sier, C. F., Stephens, R., Bizik, J., Mariani, A., Bassan, M., Pedersen, N., Frigerio, L., Ferrari, A., Dano, K., Brunner, N., Blasi, F. (1998) The level of urokinase-type plasminogen activator receptor is increased in serum of ovarian cancer patients. Cancer Res 58, 1843-1849.
(55) Gericke, B., Raila, J., Sehouli, J., Haebel, S., Konsgen, D., Mustea, A., Schweigert, F. J. (2005) Microheterogeneity of transthyretin in serum and ascitic fluid of ovarian cancer patients. BMC Cancer 5, 133.
(56) Abendstein, B., Daxenbichler, G., Windbichler, G., Zeimet, A. G., Geurts, A., Sweep, F., Marth, C. (2000) Predictive value of uPA, PAI-1, HER-2 and VEGF in the serum of ovarian cancer patients. Anticancer Res 20, 569-572.
(57) Dobryszycka, W., Warwas, M., Gerber, J., Ujec, M. (1979) Serum enzymes in ovarian carcinoma. Neoplasma 26, 737-743.
(58) Meinhold-Heerlein, I., Bauerschlag, D., Zhou, Y., Sapinoso, L. M., Ching, K., Frierson, H. F. J., Brautigam, K., Sehouli, J., Stickeler, E., Konsgen, D., Hilpert, F., von Kaisenberg, C. S., Pfisterer, J., Bauknecht, T., Jonat, W., Arnold, N., Hampton, G. M. (2007) An integrated clinical-genomics approach identifies a candidate multi-analyte blood test for serious ovarian cancer. Clin Cancer Res 13, 458-466.
(59) Moore, R. G., Brown, A. K., Miller, M. C., Skates, S., Allard, W. J., Verch, T., Steinhoff, M., Messerlian, G., DiSilvestro, P., Granai, C. O., Bast, R. C. J. (2008) The use of multiple novel tumor biomarkers for the detection of ovarian cancer in patients with pelvic mass. Gynecol Oncol 108, 402-408.
(60) Visintin, I., Feng, Z., Longton, G., Ward, D. C., Alvero, A. B., Lai, Y., Tenthorey, J., Leiser, A., Flores-Saaib, R., Yu, H., Azori, M., Rutherford, T., Schwartz, P. E., Mor, G. (2008) Diagnostic markers for early detection of ovarian cancer. Clin Cancer Res 14, 1065-1072.
(61) Ahmed, N., Oliva, K. T., Barker, G., Hoffmann, P., Reeve, S., Smith, I. A., Quinn, M. A., Rice, G. E. (2005) Proteomic tracking of serum protein isoforms as screening biomarkers of ovarian cancer. Proteomics 5, 4625-4636.
(62) Kozak, K. R., Su, F., Whitelegge, J. P., Faull, K., Reddy, S., Farias-Eisner, R. (2005) Characterization of serum biomarkers for detection of early stage ovarian cancer. Proteomics 5, 4589-4596.
(63) Isonishi, S. (2004) Biomarker in gynecologic malignancies. Gan To Kagaku Ryoho 31, 1003-1007.
(64) Vikhanskaya, F., Bani, M. R., Borsotti, P., Ghilardi, C., Ceruti, R., Ghisleni, G., Marabese, M., Giavazzi, R., Broggini, M., Taraboletti, G. (2001) p73 Overexpression increases VEGF and reduces thrombospondin-1 production: implications for tumor angiogenesis. Oncogene 20, 7293-7300.
(65) Rauvala, M., Puistola, U., Turpeenniemi-Hujanen, T. (2005) Gelatinases and their tissue inhibitors in ovarian tumors; TIMP-1 is a predictive as well as prognostic factor. Gynecol Oncol 99, 656-663.
(66) Hellstrom, I., Raycraft, J., Hayden-Ledbetter, M., Ledbetter, J. A., Schummer, M., McIntosh, M., Drescher, C., Urban, N., Hellstrom, K. E. (2003) The HE4 (WFDC2) protein is a biomarker for ovarian carcinoma. Cancer Res 63, 3695-3700.
(67) Lu, K. H., Patterson, A. P., Wang, L., Marquez, R. T., Atkinson, E. N., Baggerly, K. A., Ramoth, L. R., Rosen, D. G., Liu, J., Hellstrom, I., Smith, D., Hartmann, L., Fishman, D., Berchuck, A., Schmandt, R., Whitaker, R., Gershenson, D. M., Mills, G. B., Bast, R. C. J. (2004) Selection of potential markers for epithelial ovarian cancer with gene expression arrays and recursive descent partition analysis. Clin Cancer Res 10, 3291-3300.
(68). Kohfeldt, E., Sasaki, T., Gohring, W., Timpl, R. (1998) Nidogen-2: a new basement membrane protein with diverse binding properties. J Mol Biol 282, 99-109.
(69). Kulasingam, V., Smith, C. R., Batruch, I., Buckler, A., Jeffery, D. A., Diamandis, E. P. (2008) “Product ion monitoring” assay for prostate-specific antigen in serum using a linear ion-trap. J Proteome Res 7, 640-647.

1. A method of screening for, diagnosing or detecting ovarian cancer or an increased likelihood of developing ovarian cancer in a subject comprising:
(a) determining a level of a biomarker in a test sample from the subject wherein the biomarker is selected from the biomarkers set out in Table 2; and
(b) comparing the level of the biomarker in the test sample with a control;
wherein detecting an increased level of the biomarker in the test sample compared to the control is indicative the subject has ovarian cancer or an increased likelihood of developing ovarian cancer.

2. The method of claim 1, wherein the biomarker is nidogen-2.

3-6. (canceled)

7. The method of claim 1, wherein a ratio of the level of the biomarker in the test sample compared to the control is greater than 1, 1.2, 1.5, 1.7, 2, 3, 3, 5, 10, 12, 15, 20 or more.

8. (canceled)

9. The method of claim 2, further comprising determining the level of CA125, wherein an increased level of nidogen-2 and CA125 is indicative of ovarian cancer or an increased risk of developing ovarian cancer.

10. The method of claim 1, wherein the sample comprises blood, plasma, serum and/or ascites fluid.

11. A method for monitoring the therapeutic response of a subject with ovarian cancer comprising:
(a) determining according to claim 1, step a), a level of biomarker in a reference sample of the subject, the biomarker selected from the biomarkers set out in Table 2;
(b) determining the level of biomarker in a subsequent sample of the subject, the subsequent sample taken subsequent to the subject receiving a ovarian cancer treatment or therapy; and
(c) comparing the levels of the biomarker in the reference sample to the level of the biomarker in the subsequent sample,
wherein a decreased level of the biomarker in the subsequent sample compared to the reference sample is indicative of a positive therapeutic response and an increased level of the biomarker in the subsequent sample compared to the reference sample is indicative of a negative therapeutic response.

12. The method of claim 11 wherein the sample comprises blood, plasma, serum and/or ascites fluid.

13-15. (canceled)

16. The method of claim 11, wherein the level of the biomarker is decreased in the subsequent sample and the ratio of the level of the biomarker in the subsequent sample to the reference sample is less than 1, 0.8, 0.6, 0.4, 0.2, 0.1, 0.05, 0.001 or less.

17. (canceled)

18. The method of claim 11, wherein the level of the biomarker is increased in the subsequent sample and the ratio of the level of the biomarker in the subsequent sample compared to the reference sample is greater than 1, 1.2, 1.5, 1.7, 2, 3, 3, 5, 10, 12, 15, 20 or more.

19. (canceled)

20. The method according to claim 11, wherein the biomarker is nidogen 2.

21. The method of claim 20, further comprising determining the level of CA125, wherein a decreased level of nidogen-2 and CA125 is indicative of a positive therapeutic response and an increased level of nidogen-2 and CA125 is indicative of a negative therapeutic response.

22. The method of claim 1, wherein the ovarian cancer is a late stage ovarian cancer.

23. A method of prognosing survival in a subject with ovarian cancer comprising:
(a) determining according to claim 1, step a), a level of a biomarker in a test sample from the subject wherein the biomarker is selected from the biomarkers set out in Table 2; and
(b) comparing the level of the biomarker in the test sample with a control and/or a positive control;
wherein a decreased level of the biomarker in the test sample compared to the control is indicative of a good survival prognosis of the subject and an increased level of the biomarker in the test sample compared to the control, is indicative of a poor survival prognosis.

24-33. (canceled)

34. A method of detecting relapse in a subject previously having ovarian cancer comprising:
(a) determining according to claim 1, step a) a level of a biomarker in a test sample from the subject wherein the biomarker is selected from the biomarkers set out in Table 2; and
(b) comparing the level of the biomarker in the test sample with a control;
wherein an altered level of the biomarker in the test sample compared to the control is indicative of relapse of ovarian cancer in the subject.

35. (canceled)

36. The method according to claim 11, wherein the cancer treatment is chemotherapy optionally comprising carboplatin and/or paclitaxel.

37-42. (canceled)

43. The method of claim 34 wherein the sample comprises blood, plasma, serum and/or ascites fluid.

44-46. (canceled)

47. The method of claim 34, further comprising detecting at least one additional biomarker, wherein the additional biomarker is selected from the additional biomarkers set out in Table 1 and/or CA125.

48. The method of claim 23, wherein the sample comprises blood, plasma, serum and/or ascites fluid.

49-50. (canceled)

51. The method of claim 34, wherein the biomarker is nidogen-2.

52-55. (canceled)

56. A composition comprising two or more detection agents, wherein at least one detection agent detects a biomarker selected from the biomarkers set out in Table 2, and wherein a second detection agent detect a biomarker selected from Table 2, Table 1 and CA125, wherein the composition is used to measure the level of at least two biomarkers.

57-59. (canceled)

60. The composition of claim 56, wherein at the at least one detection agent detects nidogen-2.

61. An immunoassay for detecting a biomarker for use in a method according to claim 1 comprising an antibody or antibody fragment immobilized on a solid support, wherein the antibody binds a biomarker wherein the biomarker is selected from the biomarkers set out in Table 2.

62-63. (canceled)

64. The immunoassay of claim 61 wherein the biomarker is nidogen-2.

65. A kit comprising at least two detection agents, wherein at least one detection agent detects a biomarker selected from the biomarkers set out in Table 2; and instructions for use;
wherein the detection agents are used to measure the level of two biomarkers.

66-67. (canceled)

68. The kit of claim 65, wherein at least 1 detection agent detects nidogen-2.

69. (canceled)
